(19) United States
(12) Patent Application Publication
(10) Pub. No.: US 2022/0208173 A1
(43) Pub. Date: Jun. 30, 2022

(54) Methods of Generating Speech Using Articulatory Physiology and Systems for Practicing the Same

(71) Applicant: The Regents of the Universityy of California, Oakland, CA (US)

(72) Inventors: Edward F. Chang, San Francisco, CA (US); Gopala Krishna Anumanchipalli, Oakland, CA (US)

(21) Appl. No.: 17/603,700

(22) PCT Filed: Apr. 20, 2020
(86) PCT No.: PCT/US2020/028926

§ 371 Date: Oct. 14, 2021

Related U.S. Application Data
(60) Provisional application No. 62/879,948, filed on Jul. 29, 2019.
(60) Provisional application No. 62/837,096, filed on Apr. 22, 2019.

(51) Int. Cl. G10L 13/027; G10L 25/24; G10L 25/30; G10L 13/047; G10L 25/75; A61B 5/369; A61B 5/00; G06N 3/04

(52) U.S. Cl. CPC G10L 13/027; G10L 25/24; G10L 25/30; G06N 3/04; G10L 25/75; A61B 5/369; A61B 5/4803; G10L 13/047

(57) ABSTRACT

Provided are methods and systems of encoding and decoding speech from a subject using articulatory physiology. Methods of the present disclosure include receiving a physiological feature signal associated with a spatiotemporal movement of a vocal tract articulator, generating a speech pattern signal in response to the physiological feature signal, and outputting speech that is based on the speech pattern signal. Methods of the present disclosure further include acquiring one or more of a linguistic signal and an acoustic signal; associating a physiological feature with the linguistic or acoustic signal; generating a speech pattern signal in response to the physiological feature; and outputting speech that is based on the speech pattern signal. Speech decoding systems and devices using articulatory physiology for practicing the subject methods are also provided. Various steps and aspects of the methods will now be described in greater detail below.

### CROSS-REFERENCE TO RELATED APPLICATIONS

This application claims the benefit of U.S. Provisional Patent Application Ser. No. 62/837,096 filed Apr. 22, 2019 and U.S. Provisional Patent Application Ser. No. 62/879,948 filed Jul. 29, 2019; the disclosures of which are herein incorporated by reference in their entirety.

### INTRODUCTION

The speech signal is the result of respiratory, phonatory and articulatory processes that generate the perceivable acoustic resonances to encode an intended linguistic message. Mimicking the human system closely has remained computationally elusive and may be attributed to the lack of an imaging modality that comprehensively assays all aspects of vocal tract physiology during continuous speech making it impossible to computationally model the underlying generative processes. The strength of generative models comes from their ability to explain observed variance at its causal source. Such task specialized generative models have several useful properties such as: i) generating human like behavior; ii) reducing the reliance on endless amounts of data; iii) interpretable and swappable model components and iv) graceful degradation in unseen conditions.

There is a need for biocompatible solutions for assistive spoken communication and an opportunity in biologically inspired cognitive models for improving robustness of current speech technologies.

### SUMMARY

Provided are methods and systems of encoding and decoding speech from a subject using articulatory physiology. Methods according to certain embodiments include acquiring one or more of a phonological or acoustic signal, associating a speech pattern signal in response to the physiological feature with the phonological or acoustic signal and outputting speech that is based on the speech pattern signal. Methods according to certain embodiments include acquiring one or more of a linguistic signal and an acoustic signal; associating a physiological feature with the linguistic or acoustic signal; generating a speech pattern signal in response to the physiological feature; and outputting speech that is based on the speech pattern signal. Speech decoding systems and devices using articulatory physiology for practicing the subject methods are also provided.

### BRIEF DESCRIPTION OF THE DRAWINGS

The invention may be best understood from the following detailed description when read in conjunction with the accompanying drawings. Included in the drawings are the following figures:

FIGS. 1A-1B illustrate the Midsagittal section of the vocal tract showing select locations for EMA sensor pellets. FIG. 1B shows the graphical model of the speech production process according to certain embodiments.

FIG. 2 shows initialized physiological features for an example sentence “I'm terrible with gadgets”.

FIG. 3 illustrates an encoder-decoder network to embed physiological representation according to certain embodiments.

FIGS. 4A-4D show encoding of an unseen utterance according to certain embodiments. FIG. 4A shows a spectrogram of the speaker's original utterance. FIG. 4B shows reconstructed spectrogram after propagating through the trained stacked network. FIG. 4C shows encoded embedding of dimensions that were apriori set to be manner features. FIG. 4D shows encoded embedding of dimensions that were set to be EMA trajectories, predictions are in the solid lines and the ground truth trajectories for the utterance are also shown in dotted lines.

FIG. 5 shows a correlation across articulators on unseen utterances' EMA kinetics as depicted in Table 1.

FIG. 6 shows a comparison of two methods of speech synthesis: with and without physiological modelling, performance shown under 2 conditions of data size according to certain embodiments.

FIG. 7A-7C shows inferred Articulator Kinematics according to certain embodiments. (A) Approximate sensor locations for each articulator during EMA recordings. Midsagittal movements represented as Cartesian x and y coordinates. (B) Midsagittal articulator movements inferred from both acoustic and phonetic features (in color). The trace of each reference sensor coordinate is also shown (in black). The larynx was approximated by fundamental frequency (f0) modulated by whether the segment of speech was voiced. (C) Recorded articulator movements (EMA) representing consonants and vowels projected into a low-dimensional (LDA) space. Inferred articulator movements projected into the same space were highly correlated with the original EMA. Correlations were pairwise distances between phonemes (consonants, r=0.97, p<0.001; vowels, r=0.90, p<0.001).

FIG. 8A-8E. Neural Encoding of Articulatory Kinematic Trajectories according to certain embodiments. (A)Magnetic resonance imaging (MRI) reconstruction of single participant's brain where an example electrode is shown in the ventral sensorimotor cortex (vSMC). (B) Inferred articulator movements during the production of the phrase “stimulating discussions.” Movement directions are differentiated by color (positive x and y directions, purple; negative x and y directions, green), as shown in FIG. 7A. (C) Spatiotemporal filter resulting from fitting articulator movements to explain high gamma activity for an example electrode. Time 0 represents the alignment to the predicted sample of neural activity. (D) Convolving the spatiotemporal filter with articulator kinematics explains high gamma activity as shown by an example electrode. High gamma from 10 trials of speaking “stimulation discussions” was dynamically time warped based on the recorded acoustics and averaged together to emphasize peak high gamma activity throughout the course of a spoken phrase. (E) Example electrode-encoded filter weights projected onto a midsagittal view of the vocal tract exhibits speech-relevant articulatory kinematic trajectories (AKTs). Time course of trajectories is represented by thin-to-thick lines. Larynx (pitch modulated by voicing) is one dimensional along the y axis, with the x axis showing time course.

FIG. 9A-9C. Clustered Articulatory Kinematic Trajectories and Phonetic Outcomes according to certain embodiments. (A) Hierarchical clustering of encoded articulatory kinematic trajectories (AKTs) for all 108 electrodes across 5 participants. Each column represents one electrode. The kinematics of AKTs were described as a seven-dimensional vector by the points of maximal displacement along the principal movement axis of each articulator. Electrodes were hierarchically clustered by their kinematic descriptions resulting in four primary clusters. (B) A phoneme-encoding model was fit for each electrode. Kinematically clustered electrodes also encoded four clusters of encoded phonemes differentiated by place of articulation (alveolar, bilabial, velar, and vowels). (C) Average AKTs across all electrodes in a cluster. Four distinct vocal tract configurations encompassed coronal, labial, and dorsal constrictions in addition to vocalic control.

FIG. 10 shows Spatial Organization of Vocal Tract Gestures according to certain embodiments. Electrodes from five participants (two left and three right hemisphere) colored by kinematic cluster warped to the vSMC location on common MRI-reconstructed brain. Opacity of electrode varies with Pearson's correlation coefficient from the kinematic trajectory encoding model

FIG. 11A-11C. Damped Oscillatory Dynamics of Kinematic Trajectories according to certain embodiments. (A) Articulator trajectories from encoded AKTs along the principal movement axes for example electrodes from each kinematic cluster. Positive values indicate a combination of upward and frontward movements. (B) Articulator trajectories for all 108 encoded kinematic trajectories across 5 participants. (C) Linear relationship between peak velocity and articulator displacement (r=0.85, 0.77. 0.83, 0.69, 0.79, and 0.83 in respective order; p<0.001). Each point represents the peak velocity and associated displacement of an articulator from the AKT for an electrode

FIG. 12A-12J. Neural Representation of Coarticulated Kinematics according to certain embodiments. (A) Example of different degrees of anticipatory coarticulation for the lower incisor. Average traces for the lower incisor (y direction) are shown for /æz/ and /æp/ aligned to the acoustic onset of /æ/. (B) Electrode 120 is crucially involved in the production of /æ/ with a vocalic AKT (jaw opening and laryngeal control)and has a high phonetic selectivity index for /æ/. (C) Average high gamma activity for electrode 120 during the productions of /æbz/ and /æp/. Median high gamma during 50 ms centered at the electrode's point of peak phoneme discriminability (gray box) is significantly higher for /æp/ than /æz/ (p<0.05, Wilcoxon signed-rank tests). (D) Average predicted high gamma activity predicted by AKT in (B). Median predicted high gamma is significantly higher for /æp/ than /æz/ (p<0.001, Wilcoxon signed-rank tests). (E) Mixed-effect model shows relationship of high gamma with kinematic variability due to anticipatory coarticulatory effects of following phonemes for all electrodes and phonemes (b=0.30, SE=0.04, c2(1)=38.96, p=4e-10). Each line shows the relationship between high gamma and coarticulated kinematic variability for a given phoneme and electrode in all following phonetic contexts with at least 25 instances. Relationships from (C) and (D) for /æz/ (red) and /æp/ (yellow) are shown as points. Electrodes in all participants were used to construct the model. (F) Example of different degrees of carryover coarticulation for the lower incisor. Average traces for the lower incisor (y direction) are shown for /æz/ and /iz/ aligned to the acoustic onset of /z/. (G) Electrode 122 is crucially involved in the production of /z/ with a coronal AKT and has a high phonetic selectivity index for /z/. (H) Average high gamma activity for electrode 122 during the productions of /æz/ and /iz/. Median high gamma is significantly higher for /æz/ than /iz/ (p<0.05, Wilcoxon signed-rank tests). (I) Average predicted high gamma activity predicted by AKT in (G). Median predicted high gamma is significantly higher for /æz/ than /iz/ (p<0.001, Wilcoxon signed-rank tests). (J) Mixed-effect model shows relationship of high gamma with kinematic variability due to carryover coarticulatory effects of preceding phonemes for all electrodes (in all participants) and phonemes (b=0.32, SE=0.04, c2(1)=42.58, p=6e-11). Relationships from (H) and (I) for /æz/ (green) and /iz/ (blue) are shown as points.

FIG. 13A-13C. Neural-Encoding Model Evaluation (A) Comparison of AKT encoding performance across electrodes in different anatomical regions. Anatomical regions compared: electrodes in study (EIS), superior temporal gyms (STG), precentral gyrus* (preCG*), postcentral gyms* (postCG*), middle temporal gyrus (MTG), supramarginal gyms (SMG), pars opercularis (POP), pars triangularis (PTRI), pars orbitalis (PORB), and middle frontal gyms (MFG). Electrodes in study were speech selective electrodes from pre- and post-central gyri while preCG* and postCG* only included electrodes hat were not speech selective. EIS encoding performance was significantly higher than all other regions (p<1e-15, Wilcoxon signed-rank test). (B) Comparison of AKT- and formant-encoding models for electrodes in the study. Using F1, F2, and F3, the formant-encoding model was fit in the same manner as the AKT model. Each point represents the performance of both models for one electrode. (C) Comparison of AKT- and phonemic-encoding models. The phonemic model was fit in the same manner as the AKT model, except that phonemes were described as one hot vector. The best single phoneme predicting electrode activity was said to be the encoded phoneme of that particular electrode, and that r value was reported along with the r value of the AKT model. Pearson's r was computed on held-out data from training for all models. In both comparisons, the AKT performed significantly better (p<1e-20, Wilcoxon signed-rank test). Error bars represent SEM.

FIG. 14A-14B Decoded Articulator Movements from vSMC Activity according to certain embodiments.(A) Original (black) and predicted (colored) x and y coordinates of articulator movements during the production of an example held-out sentence. Pearson's correlation coefficient (r) for each articulator trace. (B) Average performance (correlation) for each articulator for 100 sentences held out from training set. Error bars represent SEM.

FIG. 15A-15G: Speech Synthesis from neurally decoded spoken sentences. FIG. 15A, The neural decoding process begins by extracting high-gamma amplitude (70-200 Hz) and low frequency (1-30 Hz) ECoG activity. FIG. 15B, A 3-layer bi-directional long short term memory (bLSTM) neural network learns to decode kinematic representations of articulation from filtered ECoG signals. FIG. 15C, An additional 3-layer bLSTM learns to decode acoustics from the previously decoded kinematics. Acoustics are represented as spectral features (e.g. Mel-frequency cepstral coefficients (MFCCs)) extracted from the speech waveform. FIG. 15D, Decoded signals are synthesized into an acoustic waveform. FIG. 15E, Spectrogram shows the frequency content of two sentences spoken by a participant. FIG. 15F, Spectrogram of synthesized speech from brain signals recorded simultaneously with the speech in e. Me197 cepstral distortion (MCD), a metric for assessing the spectral distortion between two audio signals, was computed for each sentence between the original and decoded audio. FIG. 15G-15H 300 ms long, median spectrograms that were time-locked to the acoustic onset of phonemes from original (FIG. 15G) and decoded (FIG. 15H) audio. Medians were computed from phonemes in 100 sentences that were withheld during decoder 101 training (n: /i/=112, /z/=102 115, /p/ 69, /ae/=86). These phonemes represent the diversity of spectral features. Original and decoded median phoneme spectrograms were well correlated (r>0.9 for all 104 phonemes, p=1e-18).

FIG. 16A-16D: Decoded speech intelligibility and feature-specific performance FIG. 16A, Spectral distortion, measured by Mel-Cepstral Distortion (MCD) (lower values are better), between original spoken sentences and neurally decoded sentences that were held out from model training (n=100). Reference MCD refers to the MCD resulting from the synthesis of original kinematics without neural decoding and provides an upper bound for performance. MCD scores were compared to chance-level MCD scores obtained by shuffling data before decoding. FIG. 16B, Decoded sentence intelligibility was assessed by asking naïve participants to identify the sentence they heard from 10 choices. Each sample (n=60) represents the percentage of correctly identified trials for one sentence. The median sentence was correctly identified 83% of the time. FIG. 16C, Correlation of original and decoded spectral features. Values represent the mean correlation 166 of the 32 spectral features for each sentence (n=100). Correlation performance for individual spectral features is reported in extended data FIG. 1b. FIG. 16D, Correlations between original and decoded intelligibility-relevant features. Kinematic values represent the mean correlation of the 33 kinematic features (the intermediate representation) for each sentence (n=100). Correlation performance for individual kinematic features is reported in FIG. 19A. Box plots depict median (horizontal line inside box), 25th and 75th percentiles (box), 25/75th percentiles ±1.5× interquartile range (whiskers), and outliers (circles). Distributions were compared with each as other as indicated or with chance-level distributions using two-tailed Wilcoxon signed-rank tests (p<1e-10, n=100, for all 176 tests).

FIGS. 17A-17F: Effects of model design decisions. FIG. 17A-17B, Mean correlation of original and decoded spectral features (FIG. 3A) and mean spectral distortion (MCD) (FIG. 17B) for model trained on varying amounts of training data. Training data was split according to recording session boundaries resulting the following sizes: 2.4, 5.2, 12.6, 25.3, 44.9, 55.2, 77.4, and 92.3 minutes of speaking data. The neural decoding approach that included an articulatory intermediate stage (purple) performed significantly better with every size of training data than direct ECoG to acoustics decoder (grey) (all: p<1e-5, n=100; Wilcoxon signed-rank test, error bars=SE). FIG. 17C, Acoustic similarity matrix compares acoustic properties of decoded phonemes and originally spoken phonemes. Similarity is computed by first estimating a gaussian kernel density for each phoneme (both decoded and original) and then computing the Kullback-Leibler (KL) divergence between a pair of decoded and original phoneme distributions. Each row compares the acoustic properties of a decoded phoneme with originally spoken phonemes (columns). Hierarchical clustering was performed on the resulting similarity matrix. FIG. 17D, Anatomical reconstruction of a single participant's brain with the following regions used for neural decoding: ventral sensorimotor cortex (vSMC), superior temporal gyrus (STG), and inferior frontal gyrus (IFG). FIG. 17E-17F, Difference in spectral distortion (MCD) (FIG. 17E), and difference in correlation (Pearson's r) performance (FIG. 17F) between decoder 248 trained on all regions and decoders trained on all-but-one region. Exclusion of any region resulted in decreased performance (p<3e-4, n=100; Wilcoxon signed-rank test). Box plots as described in FIG. 16.

FIGS. 18A-18E: Speech synthesis from neural decoding of silently mimed speech. FIGS. 18A-18C, Spectrograms of original spoken sentence (a), neural decoding from audible production (FIG. 18B), and neural decoding from silently mimed production (c). FIG. 18D-18E, Spectral distortion (MCD) (FIG. 18D) and correlation of original and decoded spectral features (FIG. 18E) for audibly and silently produced speech. Since correlations are with respect to original audibly produced sentences, decoded sentences that were silently mimed were dynamically time-warped according to their spectral features. Decoded sentences were significantly better than chance-level decoding for both speaking conditions (p<1e-11, for all comparisons, n=58; Wilcoxon signed-rank test). Box plots as described in FIG. 16.

FIGS. 19A-19B: Decoding performance of kinematic and spectral features. FIG. 19A Correlations of all 33 decoded articulatory kinematic features with ground-truth. EMA features represent X and Y coordinate traces of articulators (lips, jaw, and three points of the tongue) along the midsagittal plane of the vocal tract. Manner features represent complementary kinematic features to EMA that further describe acoustically consequential movements. FIG. 19B, Correlations of all 32 decoded spectral features with ground-truth. MFCC features are 25 mel-frequency cepstral coefficients that describe power in perceptually relevant frequency bands. Synthesis features describe glottal excitation weights necessary for speech synthesis.

FIG. 20: Ground-truth acoustic similarity matrix. Compares acoustic properties of ground-truth spoken phonemes with one another. Similarity is computed by first estimating a gaussian kernel density for each phoneme and then computing the Kullback-Leibler (KL) divergence between a pair of a phoneme distributions. Each row compares the acoustic properties of a two ground-truth spoken phonemes. Hierarchical clustering was performed on the resulting similarity matrix.

### DETAILED DESCRIPTION

Provided are methods and systems of encoding and decoding speech from a subject using articulatory physiology. Methods of the present disclosure include receiving a physiological feature signal associated with a spatiotemporal movement of a vocal tract articulator, generating a speech pattern signal in response to the physiological feature signal, and outputting speech that is based on the speech pattern signal. Methods of the present disclosure further include acquiring one or more of a linguistic signal and an acoustic signal; associating a physiological feature with the linguistic or acoustic signal; generating a speech pattern signal in response to the physiological feature; and outputting speech that is based on the speech pattern signal. Speech decoding systems and devices using articulatory physiology for practicing the subject methods are also provided. Various steps and aspects of the methods will now be described in greater detail below.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “an electrode” includes a plurality of such electrodes and reference to “the signal” includes reference to one or more signals, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflict with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### METHODS—ENCODING AND DECODING SPEECH USING ARTICULATORY PHYSIOLOGY

As summarized above, aspects of the invention include methods and systems of encoding and decoding speech from a subject using articulatory physiology. Methods of the present disclosure include receiving a physiological feature signal associated with a spatiotemporal movement of a vocal tract articulator, generating a speech pattern signal in response to the physiological feature signal, and outputting speech that is based on the speech pattern signal. Methods of the present disclosure further include acquiring one or more of a linguistic signal and an acoustic signal; associating a physiological feature with the linguistic or acoustic signal; generating a speech pattern signal in response to the physiological feature; and outputting speech that is based on the speech pattern signal. Speech decoding systems and devices using articulatory physiology for practicing the subject methods are also provided. Various steps and aspects of the methods will now be described in greater detail below.

Aspects of the present disclosure include a method comprising receiving a physiological feature signal associated with a spatiotemporal movement of a vocal tract articulator; generating a speech pattern signal in response to the physiological feature signal; and outputting speech that is based on the speech pattern signal.

In some embodiments, the vocal tract articulator provides for spatiotemporal movements of a portion of the body associated with the vocal tract. Examples of a vocal tract articulator include the upper lip, lower lip, lower incisor, tongue tip, tongue blade, tongue dorsum, and/or larynx. For example, the wide range of spoken sounds results from highly flexible configurations of the vocal tract, which filters sound product at, for example, the larynx, via precisely coordinated movements of the lips, jaw, and tongue. Each vocal tract articulator has extensive degrees of freedom, allowing a large number of different realizations for speech movements. Examples of spatiotemporal representation of articulators is described in U.S. Pat. No. 9,905,239, which is hereby incorporated by reference in its entirety.

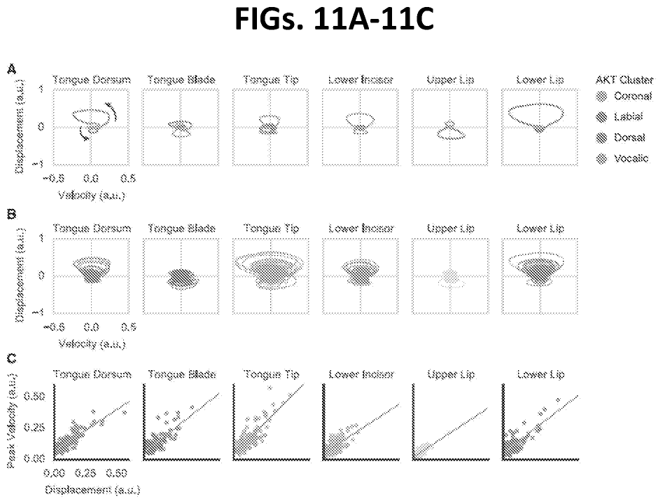

In some embodiments, the physiological feature signals comprise measurements of the caudo-rostral displacements of one or more of the vocal tract articulators. In some embodiments, the method comprises measuring the caudo-rostral displacements of the one or more of the vocal tract articulators associated with consonant constriction. In some embodiments, the caudo-rostral displacements capture the shaping of the vocal tract and places of articulation. In some embodiments, the consonant constriction determines whether the consonant is a plosive, lateral, fricative, or nasal consonant. In some embodiments, the measured caudo-rostral displacements range from 0-0.1, 0.1-0.2, 0.2-0.3, 0.3-0.4, 0.4-0.5, 0.5-0.6, 0.6-0.7, 0.7-0.8, 0.8-0.9, or 0.9-1.0 a.u. In some embodiments, the measured caudo-rostral displacements range from 0-0.25, 0.25-0.5, 0.5-1.0 a.u. In some embodiments, the physiological feature signals comprise measurements of the caudo-rostral velocity. In some embodiments, the measured caudo-rostral velocity range from 0-0.1, 0.1-0.2, 0.2-0.3, 0.3-0.4, 0.4-0.5, 0.5-0.6, 0.6-0.7, 0.7-0.8, 0.8-0.9, or 0.9-1.0 a.u. In some embodiments, the measured caudo-rostral velocity range from 0-0.25, 0.25-0.5, 0.5-1.0 a.u. In some embodiments, the physiological feature signals comprise measurements of the caudo-rostral displacement and velocity. In some embodiments, the measured velocity is proportional to the measured displacement (see e.g. FIG. 11A-11C).

In some embodiments, spatiotemporal movement of a vocal tract articulator is measured by electromagnetic midsagittal articulography. Vocal tract imaging technique using electromagnetic midsagittal articulography can be used study articulation during continuous speech production. The term “Electromagnetic midsagittal articulography” (EMA) is used herein in its conventional sense to refer to a kinematic tracking system that uses low field-strength electromagnetic fields to measure the movement of the portions of the body associated with the vocal tract (e.g. tongue, lips, jaw, and/or velum). In some embodiments, two-dimensional (2D) EMA measures movement in the midsagittal plane. In certain instances, subjects wear a helmet that places three transmitter coils around the head. The transmitters produce alternating magnetic fields which generate currents in tiny sensors placed on the surface of the articulators. As the sensors move through the fields, they are tracked by computer.

In some embodiments, receiving a physiological feature signal comprises receiving one or more brain signals; and associating the brain signals to one or more of the spatiotemporal movements of a vocal tract articulator. In some embodiments, the signals are detected from the cortical region of the brain. In some embodiments, the signals are detected from the ventral sensorimotor cortex of the brain. In some embodiments, the signals are neural signals. In some embodiments, the neural (e.g. brain signals) signals are detected by contacting an electrocorticography (ECoG) electrode array with the cortical region of the brain in an individual. In some embodiments, the signals are acquired by contacting 1 or more electrodes, 2 or more electrodes, or 3 or more electrodes that detect the plurality of signals with at least one region of the brain. In some embodiments, the signals are acquired by contacting 50 or more electrodes, 100 or more electrodes, 150 or more electrodes, 200 or more electrodes, 250 or more electrodes, or 300 or more electrodes that detect the signals with at least one region of the brain. In some embodiments, the at least one region of the brain comprises the speech motor cortex of the brain.

In some embodiments, the method comprises acquiring brains signals (e.g. ECoG signals), with an electrical recording device configured to record ECoG signals in the brain. In some embodiments, the electrical recording device is an ECoG 128-channel recording device. In some embodiments, the electrical recording device is an ECoG 256-channel recording device. In some embodiments, the electrical recording device is implantable. In some embodiments, the electrical recording device is wireless.

In some embodiments, the method comprises receiving and/or acquiring brain signals. In some embodiments, the ECoG signals are filtered in a high gamma frequency range to obtain neural signals in the auditory and sensorimotor brain regions. In some embodiments, plurality of signals are obtained from auditory and sensorimotor brain regions selected from the vSMC, STG, and IFG. In some embodiments, plurality of signals are obtained from auditory and sensorimotor brain regions from the vSMC. In some embodiments, the signals comprise the high-gamma frequency component and/or the local field potentials. The high-gamma frequency component is a high-gamma frequency range of the signals associated one or more of the spatiotemporal movements of a vocal tract articulator t. In some embodiments, the high-gamma frequency range ranges from 70-200 Hz (e.g. 70-75 Hz, 75-80 Hz, 80-85 Hz, 95-90 Hz, 90-95 Hz, 95-100 Hz, 100-105 Hz, 105-110 Hz, 110-115 Hz, 115-120 Hz, 120-125 Hz, 125-130 Hz, 130-135 Hz, 135-140 Hz, 140-145 Hz, 145-150 Hz, 150-155 Hz, 155-160 Hz, 160-165 Hz, 165-170 Hz, 170-175 Hz, 175-180 Hz, 180-185 Hz, 185-190 Hz, 190-195 Hz, or 195-200 Hz). In some embodiments, the high-gamma frequency range ranges from 70-150 Hz.

In some embodiments, the signals are detected using at least three electrodes operably coupled to the speech motor cortex of the subject. By “operably coupled” is meant that one or more electrodes are of a suitable type and position so as to detect the desired signals in the speech motor cortex associated with one or more of the spatiotemporal movements of a vocal tract articulator. According to one embodiment, the one or more electrodes are operably coupled to the speech motor cortex by implantation on the surface of the speech motor cortex. In one aspect, an array of electrocorticography electrodes (ECoG array) is disposed on the surface of the speech motor cortex (e.g., the vSMC) for detection of neural signals (e.g., local field potentials and/or high gamma frequency signals) generated in the speech motor cortex. In some embodiments, the neural signals comprise local field potentials generated in the speech motor cortex. In some embodiments, the signals comprise high gamma frequency signals (e.g. 70-200 Hz) generated in the speech motor cortex. In some embodiments, the signals comprise spectral features of the neural signals. In some embodiments, the spectral features are Mel-frequency cepstral coefficients (MFCCs) extracted from the speech waveform (e.g. local field potentials generated in the speech motor cortex). According to certain embodiments, the one or more electrodes are operably coupled to the speech motor cortex by insertion of the electrodes into the speech motor cortex (e.g., at a desired depth). According to certain embodiments, the ECoG electrode array is implantable. According to certain embodiments, the ECoG electrode array is implanted directly on the surface of the brain.

An array may include, for example, about 5 electrodes or more, e.g., about 5 to 10 electrodes, about 10 to 20 electrodes, about 20 to 30 electrodes, about 30 to 40 electrodes, about 40 to 50 electrodes, about 50 to 60 electrodes, about 60 to 70 electrodes, about 70 to 80 electrodes, about 80 to 90 electrodes, about 90 to 100 electrodes, about 100 to 125 electrodes, about 125 to 150 electrodes, about 150 to 200 electrodes, about 200 to 250 electrodes, about 250 to 300 electrodes (e.g., a 256 electrode array in 16×16 format), about 300 to 400 electrodes, about 400 to 500 electrodes, or about 500 electrodes or more. In some embodiments, the array includes a 256 electrode array in 16×16 format. In certain embodiments, the array may cover a surface area of about 1cm², about 1 to 10 cm², about 10 to 25 cm², about 25 to 50 cm², about 50 to 75 cm², about 75 to 100 cm², or 100 cm² or more. Arrays of interest may include, but are not limited to, those described in U.S. Pat. Nos. USD565735; USD603051; USD641886; and USD647208; the disclosures of which are incorporated herein by reference.

The specific location at which to position an electrode may be determined by identification of anatomical landmarks in the subject's brain, such as the pre-central and post-central gyri and the central sulcus. For example, in some embodiments, the location of the electrode is at or near the precentral and/or postcentral gyri that has distinguishable high gamma activity during speech production were selected Identification of anatomical landmarks in a subject's brain may be accomplished by any convenient means, such as magnetic resonance imaging (MRI), functional magnetic resonance imaging (fMRI), and visual inspection of a subject's brain while undergoing a craniotomy. Once a suitable location for an electrode is determined, the electrode may be positioned (e.g., implanted) according to any convenient means. Suitable locations for positioning or implanting the at least three electrodes may include, but are not limited to, one or more regions of the ventral sensorimotor cortex (vSMC), including the pre-central gyrus, the post-central gyrus, the guenon (the gyral area directly ventral to the termination of the central sulcus), the superior temporal gyrus (STG), the inferior frontal gyms (IFG), and any combination thereof. Correct placement of the at least three electrodes may be confirmed by any convenient means, including visual inspection or computed tomography (CT) scan. In some aspects, after electrode positions are confirmed, they may be superimposed on a surface reconstruction image of the subject's brain. In certain aspects, the electrodes are positioned such that the ECoG signals are detected from one or more regions of the vSMC, e.g., the ECoG signals are detected from a region of the vSMC selected from the pre-central gyms, the post-central gyrus, the guenon, STG, IFG, and combinations thereof.

Methods of interest for positioning electrodes further include, but are not limited to, those described in U.S. Pat. Nos. 4,084,583; 5,119,816; 5,291,888; 5,361,773; 5,479,934; 5,724,984; 5,817,029; 6,256,531; 6,381,481; 6,510,340; 7,239,910; 7,715,607; 7,908,009; 8,045,775; and 8,019,142; the disclosures of which are incorporated herein by reference in their entireties for all purposes.

The number of electrodes operably coupled to the speech motor cortex may be chosen so as to provide the desired resolution and information about the neural signals being generated in the speech motor cortex.

In some embodiments, the method includes generating a speech pattern signal in response to the physiological feature signal. In some embodiments, the speech pattern signal comprises a combination of phonological, physiological, and acoustic signals. In some embodiments, the method includes outputting speech that is based on the speech pattern signal. In some embodiments, the speech pattern signal is outputted as auditory speech or as text. In some embodiments, the auditory speech can be sounds of one or more syllables, words, parts of words, phrases, utterances, paragraphs, sentences, and/or a combination thereof. In some embodiments, the text includes one or more syllables, words, parts of words, phrases, utterances, paragraphs, sentences, and/or a combination thereof.

Aspects of the present disclosure include methods comprising: acquiring one or more of: a linguistic signal; and an acoustic signal; associating a physiological feature with the linguistic or acoustic signal; generating a speech pattern signal in response to the physiological feature; and outputting speech that is based on the speech pattern signal. In some embodiments, the phonological level describes the signal in terms of phonemes, syllables and their properties. In some embodiments, the acoustic signal comprises a continuous time domain or spectrotemporal representation of the acoustic resonances as produced, e.g. by a subject.

Speech communication process is cognitively symbolic (i.e., lexical and phonological) within the speaker and the listener, the underlying phonological string uttered by a speaker is realized and executed as a continuous motor sequence where the ventral sensorimotor cortex mediates the coarticulated, multi-articulator spatiotemporal movements of the vocal tract articulators. These physiological movements add higher order resonances to the acoustic source of air expelled through vibrating vocal cords. The resulting acoustic signal is then perceived by the listener in the auditory cortex in terms of the phonetic features of the incoming acoustic stream.

In some embodiments, the linguistic signal is a lexical signal. In some embodiments, the linguistic signal is a phonological signal.

In some embodiments, associating a physiological feature with the linguistic or acoustic signal comprises associating the linguistic or acoustic signal with the spatiotemporal movement of the vocal tract articulator. In some embodiments, associating a physiological feature with the linguistic or acoustic signal comprises associating the linguistic or acoustic signal with a spatiotemporal movement of a vocal tract articulator. In some embodiments, associating a physiological feature with an acoustic signal includes associating the acoustic signal with the spatiotemporal movement of the vocal tract articulator. As described above, examples of the vocal tract articulator can include the upper lip, lower lip, lower incisor, tongue tip, tongue blade, tongue dorsum, and/or larynx. In some embodiments, the vocal tract articulator is within the oropharyngeal and nasal cavity.

In some embodiments, the method comprises measuring the caudo-rostral displacements of one or more of the vocal tract articulators. In some embodiments, the method comprises measuring the caudo-rostral displacements of one or more of the vocal tract articulators associated with consonant constriction. In some embodiments, the consonant is plosive, lateral, fricative, or nasal.

In some embodiments, associating a physiological feature with the linguistic or acoustic signal further comprises detecting one or more signals from the brain; and associating the brain signals to one or more spatiotemporal movements of the vocal tract articulator. In some embodiments, the signals are detected from the ventral sensorimotor cortex of the brain.

In some embodiments, the method includes generating a speech pattern signal in response to the physiological feature signal. In some embodiments, the method includes outputting speech that is based on the speech pattern signal. In some embodiments, the speech pattern signal is outputted as auditory speech or as text. In some embodiments, the auditory speech can be sounds of one or more syllables, words, parts of words, phrases, utterances, paragraphs, sentences, and/or a combination thereof. In some embodiments, the text comprises one or more syllables, words, parts of words, phrases, utterances, paragraphs, sentences, and/or a combination thereof.

In some embodiments, the method comprises measuring the caudo-rostral displacements of one or more of the vocal tract articulators.

In some embodiments, comprises measuring the caudo-rostral displacements of one or more of the vocal tract articulators associated with consonant constriction. In some embodiments, the consonant is plosive, lateral, fricative or nasal.

In some embodiments, the spatiotemporal movement of a vocal tract articulator is measured by EMA.

In some embodiments, associating a physiological feature with the linguistic or acoustic signal further comprises: detecting one or more signals from the brain; and associating the brain signals to one or more spatiotemporal movements of a vocal tract articulator. In some embodiments, the signals are detected from the ventral sensorimotor cortex of the brain.

Phonemes by definition are segmental, perceptually defined, discrete units of sound. By “speech output” is meant a phonetic component of a word (e.g., a phoneme, a formant (e.g., formant acoustics of a vowel(s)), a diphone, a triphone, a syllable (such as a consonant-to-vowel transition (CV)), two or more syllables, a word (e.g., a single-syllable or multi-syllable word), a phrase, a sentence, or any combination of such speech sounds. The term “phoneme”, used in its conventional sense to refer to segmental, perceptually defined, discrete units of sound.

In certain aspects, the speech sound includes speech information such as formants (e.g., spectral peaks of the sound spectrum |P(f)| of the voice) and pitch (e.g., how “high” or “low” the speech sound is depending on the rate of vibration of the vocal chords) which are encoded in the speech production signals and capable of being decoded from the detected speech production signals and/or patterns thereof. For example, with respect to formants, the speech production signals (e.g., vSMC activity) correlate to the acoustic parameters of different vowels, as well as different renditions of the same vowel.

Deriving a speech pattern signal in response to the physiological feature may be performed using any suitable approach. For example, the speech pattern signals may be generated for a desired duration of time by associating one or more signals from the brain with the physiological feature or physiological feature signal as measured by EMA that is associated with a spatiotemporal movement of a vocal tract articulator or with a linguistic or acoustic signal.

Multichannel population neural signals (e.g. one or more brain signals) may be analyzed using methods including, but not limited to, general linear regression, correlation, linear quadratic estimation (Kalman-Bucy filter), dimensionality reduction (e.g., principal components analysis), clustering, pattern classification, and combinations thereof.

According to certain embodiments, method can be used for a subject that have a speech impairment or inability to communicate. Subjects of interest include, but are not limited to, subjects suffering from paralysis, locked-in syndrome, Lou Gehrig' s disease, aphasia, dysarthria, stuttering, laryngeal dysfunction/loss, vocal tract dysfunction, and the like.

In certain aspects, the methods of the present disclosure further include producing the speech sound in audible form (e.g., through a speaker), displaying the speech sound in text format (e.g., on a display), or both.

### Methods—Speech Synthesis Decoding

Provided are methods of decoding speech from the brain of a subject. The methods include decoding neural signals detected from electrodes operably coupled to the speech motor cortex of an individual and extracting speech-related features from the neural signals when an individual is intended to produce a speech output in order to decode the intended speech output from the neural signals. The methods further include decoding articulatory movement features from one or more features of the neural signals into acoustic signals and decoding the acoustic signals into a speech output. The methods further include decoding auditory perceived speech or verbal produced speech in an individual into one or more syllables, words, parts of words, phrases, utterances, paragraphs, sentences, and/or a combination thereof. Speech decoding systems and devices for practicing the subject methods are also provided.

Aspects of the present disclosure include a method of decoding speech events in an individual. In some embodiments, the method includes extracting speech-related features from a plurality of signals from the brain of an individual when the individual is intended to produce a speech output; and decoding with one or more decoding constraints the intended speech output from the plurality of signals.

In some embodiments, silent speech comprises making mouthing movements without producing an audible sound.

Intended speech can include “perceived” or “attempted” speech production and is used interchangeably herein. In some embodiments, context priors can be used to for decoding perceived or produced speech. Non-limiting examples of “perceived” speech can include predicted speech before a speech output is produced from the vocal tract in the individual. Non-limiting examples of “perceived” speech can include attempted speech before a speech output is produced from the vocal tract in the individual. In some embodiments, the methods of the present disclosure provide for decoding predicted speech output before a produced speech output. In some embodiments, the methods of the present disclosure provide for decoding predicted speech output at approximately five seconds or more, approximately ten seconds or more, approximately thirty seconds or more, approximately forty seconds or more, approximately fifty seconds or more, approximately one minute or more, approximately two minutes or more, approximately three minutes or more, approximately four minutes or more, or approximately five minutes or more before a produced speech output. In some embodiments, the methods of the present disclosure provide for decoding predicted speech output at five seconds or more, ten seconds or more, twenty seconds or more, thirty seconds or more, forty seconds or more, fifty seconds or more, one minute or more, two minutes or more, three minutes or more, four minutes or more, or five minutes or more before a produced speech output. In some embodiments, “produced” speech comprises one or more syllables, words, parts of words, phrases, utterances, paragraphs, parts of paragraphs, sentences, parts of sentences, and/or a combination thereof that produce an audible sound. In some embodiments, the methods of the present disclosure provide for decoding a produced speech output at approximately five seconds or more, approximately ten seconds or more, approximately twenty seconds or more, approximately thirty seconds or more, approximately forty seconds or more, approximately fifty seconds or more, approximately one minute or more, approximately two minutes or more, approximately three minutes or more, approximately four minutes or more, or approximately five minutes or more after a produced speech output. In some embodiments, the methods of the present disclosure provide for decoding a produced speech output at five seconds or more, ten seconds or more, thirty seconds or more, forty seconds or more, fifty seconds or more, one minute or more, two minutes or more, three minutes or more, four minutes or more, or five minutes or more after a produced speech output.

Aspects of the present disclosure further include methods of decoding auditory perceived speech or verbal produced speech in an individual, the method comprising: contacting an electrode array with the cortical region of the brain in the individual; conducting speech perception training on the individual, wherein speech perception training comprises listening to pre-recorded questions; conducting speech production training on the individual, wherein speech production training comprises reading one or more answers on a screen; conducting speech testing on the individual, wherein speech testing comprises listening to pre- recorded questions and responding verbally with answers to the pre-recorded questions; recording a time-aligned audio of the speech perception training, speech production training, and speech testing on the individual; recording neurophysiological signals; analyzing the neurophysiological signals in the cortical region of the brain; and decoding the neurophysiological signals into a speech output.

In some embodiments, the methods of the present disclosure comprise generating decoding constraints by conducting one or more external context-related cues. In some embodiments, the one or more external context-related cues includes listening to one or more questions. In some embodiments, the one or more questions are pre-recorded questions. In some embodiments, the one or more external context-related cues comprises reading one or more answers on a screen. In some embodiments, the one or more external context-related cues comprises reading aloud one or more syllables, words, parts of words, phrases, utterances, paragraphs, sentences, and/or a combination thereof. In some embodiments, the one or more external context-related cues comprises reading, aloud, one or more scripts. In some embodiments, the one or more external context-related cues comprises verbally producing a set of answer responses after listening to the one or more questions. In some embodiments, the one or more external context-related cues comprises reading one or more answers on a screen. In some embodiments, the one or more external context-related cues comprises responding to one or more questions. In some embodiments, responding to one or more questions comprises a verbal response. In some embodiments, responding to one or more questions comprises a silently mimed response. In some embodiments, the one or more external context-related cues comprises silently mimed speech. In some embodiments, the one or more external context-related cues comprises silently miming one or more syllables, words, parts of words, phrases, utterances, paragraphs, sentences, and/or a combination thereof by making the kinematic movements of a verbal response but without making sound. In some embodiments, the kinematic movements during the silently mimed speech is recorded (e.g. in the form of acoustic signals). In some embodiments, the kinematic movements are correlated with recorded acoustic signals. In some embodiments, the one or more external context-related cues comprises a verbal response. In some embodiments, the verbal response is a sound. In some embodiments, the sound is selected from the group consisting of: a phoneme, formant acoustics of a vowel, a diphone, a triphone, a consonant-vowel transition, a syllable, a word, a phrase, a sentence, and combinations thereof.

In some embodiments, the data produced (e.g. neural signals, optical signals, audio recordings) from the one or more external context-related cues serve as input to train speech detection and decoding models of the present disclosure.

Aspects of the present disclosure include detecting a plurality of neurophysiological signals from the cortical region of the brain. In some embodiments, the plurality of neurophysiological signals are neural signals. In some embodiments, the plurality of neurophysiological signals are optical signals. In some embodiments, the neurophysiological signals are acquired by contacting 1 or more electrodes, 2 or more electrodes, or 3 or more electrodes that detect the plurality of signals with at least one region of the brain. In some embodiments, the neurophysiological signals are acquired by contacting 50 or more electrodes, 100 or more electrodes, 150 or more electrodes, 200 or more electrodes, 250 or more electrodes, or 300 or more electrodes that detect the plurality of signals with at least one region of the brain. In some embodiments, the at least one region of the brain comprises the speech motor cortex of the brain.

The neurophysiological signals are detected using at least three electrodes operably coupled to the speech motor cortex of the subject. By “operably coupled” is meant that one or more electrodes are of a suitable type and position so as to detect the desired neurophysiological signals in the speech motor cortex related to a speech event. According to one embodiment, the one or more electrodes are operably coupled to the speech motor cortex by implantation on the surface of the speech motor cortex. In one aspect, an array of electrocorticography electrodes (ECoG array) is disposed on the surface of the speech motor cortex (e.g., the vSMC) for detection of ECoG neural signals (e.g., local field potentials) generated in the speech motor cortex. In some embodiments, the method comprises extracted speech-related features from the neural or optical signals. In some embodiments, the speech- related features comprise local field potentials generated in the speech motor cortex. In some embodiments, the speech-related features comprise high gamma frequency signals (e.g. 70-200 Hz) generated in the speech motor cortex. In some embodiments, the speech-related features comprise spectral features of the neural signals. In some embodiments, the spectral features are Mel-frequency cepstral coefficients (MFCCs) extracted from the speech waveform (e.g. local field potentials generated in the speech motor cortex). According to certain embodiments, the one or more electrodes are operably coupled to the speech motor cortex by insertion of the electrodes into the speech motor cortex (e.g., at a desired depth). According to certain embodiments, the neurophysiological electrode array is implantable. According to certain embodiments, the neurophysiological electrode array is implanted directly on the surface of the brain.

The specific location at which to position an electrode may be determined by identification of anatomical landmarks in the subject's brain, such as the pre-central and post- central gyri and the central sulcus. Identification of anatomical landmarks in a subject's brain may be accomplished by any convenient means, such as magnetic resonance imaging (MRI), functional magnetic resonance imaging (fMRI), and visual inspection of a subject's brain while undergoing a craniotomy. Once a suitable location for an electrode is determined, the electrode may be positioned (e.g., implanted) according to any convenient means. Suitable locations for positioning or implanting the at least three electrodes may include, but are not limited to, one or more regions of the ventral sensorimotor cortex (vSMC), including the pre- central gyrus, the post-central gyrus, the guenon (the gyral area directly ventral to the termination of the central sulcus), the superior temporal gyms (STG), the inferior frontal gyms (IFG), and any combination thereof. Correct placement of the at least three electrodes may be confirmed by any convenient means, including visual inspection or computed tomography (CT) scan. In some aspects, after electrode positions are confirmed, they may be superimposed on a surface reconstruction image of the subject's brain. In certain aspects, the electrodes are positioned such that the neurophysiological signals are detected from one or more regions of the vSMC, e.g., the neurophysiological signals are detected from a region of the vSMC selected from the pre-central gyrus, the post-central gyrus, the guenon, STG, IFG, and combinations thereof.

Methods of interest for positioning electrodes further include, but are not limited to, those described in U.S. Pat. Nos. 4,084,583; 5,119,816; 5,291,888; 5,361,773; 5,479,934; 5,724,984; 5,817,029; 6,256,531; 6,381,481; 6,510,340; 7,239,910; 7,715,607; 7,908,009; 8,045,775; and 8,019,142; the disclosures of which are incorporated herein by reference in their entireties for all purposes.

The number of electrodes operably coupled to the speech motor cortex may be chosen so as to provide the desired resolution and information about the neurophysiological neural signals being generated in the speech motor cortex during one or more external context- related cues, as each electrode may convey information about the activity of a particular region (e.g., the vSMC, STG, or IFG as described in the examples below). By comparing differences between the signals of each electrode, neurophysiological neural signal patterns may be derived from the neural signals, or which electrodes responsive to the speech perception or speech production.

Accordingly, in certain embodiments, at least 10 electrodes (e.g., at least 20 electrodes) are employed. Between about 3 and 1024 electrodes, or more, may be employed. In some embodiments, the number of electrodes positioned is about 3 to 10 electrodes, about 10 to 20 electrodes, about 20 to 30 electrodes, about 30 to 40 electrodes, about 40 to 50 electrodes, about 60 to 70 electrodes, about 70 to 80 electrodes, about 80 to 90 electrodes, about 90 to 100 electrodes, about 100 to 110 electrodes, about 110 to 120 electrodes, about 120 to 130 electrodes, about 130 to 140 electrodes, about 140 to 150 electrodes, about 150 to 160 electrodes, about 160 to 170 electrodes, about 170 to 180 electrodes, about 180 to 190 electrodes, about 190 to 200 electrodes, about 200 to 210 electrodes, about 210 to 220 electrodes, about 220 to 230 electrodes, about 230 to 240 electrodes, about 240 to 250 electrodes, about 250 to 300 electrodes (e.g., a 16x16 array of 256 electrodes), about 300 to 400 electrodes, about 400 to 500 electrodes, about 500 to 600 electrodes, about 600 to 700 electrodes, about 700 to 800 electrodes, about 800 to 900 electrodes, about 900 to 1000 electrodes, or about 1000 to 1024 electrodes, or more. The electrodes may be homogeneous or heterogeneous.

Electrodes may be arranged in no particular pattern or any convenient pattern to facilitate detection of neural signals. For example, a plurality of electrodes may be placed in a grid pattern, in which the spacing between adjacent electrodes is approximately equivalent. Such spacing between adjacent electrodes may be, for example, about 2.5 cm or less, about 2 cm or less, about 1.5 cm or less, about 1 cm or less, about 0.5cm or less, about 0.1 cm or less, or about 0.05 cm or less. Electrodes placed in a grid pattern may be arranged such that the overall plurality of electrodes forms a roughly geometrical shape. In certain embodiments, a grid pattern may be roughly square in overall shape, roughly rectangular, roughly trapezoidal, or roughly oval in shape, or roughly circular.

Electrodes may be pre-arranged into an array, such that the array includes a plurality of electrodes that may be placed on or in a subject's brain. Such arrays may be miniature- or micro-arrays, a non-limiting example of which may be a miniature neurophysiological array (e.g. ECoG array, microelectrode array, electroencephalography (EEG), array). For a general review of ECoG technology, see Ajmone-Marsan, C. Electrocorticography: Historical Comments on its Development and the Evolution of its Practical Applications, Electroencephalogr. Clin. Neurophysiol, Suppl. 1998, 48: 10-16; the disclosure of which is incorporated herein by reference.

Also of interest are electrodes that may receive electroencephalography (EEG) data. One or more wet or dry EEG electrodes may be used in practicing the subject methods. Electrodes and electrode systems of interest further include, but are not limited to, those described in U.S. Patent Publication Numbers 2007/0093706, 2009/0281408, 2010/0130844, 2010/0198042, 2011/0046502, 2011/0046503, 2011/0046504, 2011/0237923, 2011/0282231, 2011/0282232 and U.S. Pat. Nos. 4,709,702, 4,967,038, 5,038,782, 6,154,669; the disclosures of which are incorporated herein by reference.

An array may include, for example, about 5 electrodes or more, e.g., about 5 to 10 electrodes, about 10 to 20 electrodes, about 20 to 30 electrodes, about 30 to 40 electrodes, about 40 to 50 electrodes, about 50 to 60 electrodes, about 60 to 70 electrodes, about 70 to 80 electrodes, about 80 to 90 electrodes, about 90 to 100 electrodes, about 100 to 125 electrodes, about 125 to 150 electrodes, about 150 to 200 electrodes, about 200 to 250 electrodes, about 250 to 300 electrodes (e.g., a 256 electrode array in 16x16 format), about 300 to 400 electrodes, about 400 to 500 electrodes, or about 500 electrodes or more. In certain embodiments, the array may cover a surface area of about 1cm², about 1 to 10 cm², about 10 to 25 cm², about 25 to 50 cm², about 50 to 75 cm², about 75 to 100 cm², or 100 cm² or more. Arrays of interest may include, but are not limited to, those described in U.S. Pat. Nos. USD565735; USD603051; USD641886; and USD647208; the disclosures of which are incorporated herein by reference.

Electrodes may be platinum-iridium electrodes or be made out of any convenient material. The diameter, length, and composition of the electrodes to be employed may be determined in accordance with routine procedures known to those skilled in the art. Factors which may be weighted when selecting an appropriate electrode type may include but not be limited to the desired location for placement, the type of subject, the age of the subject, cost, duration for which the electrode may need to be positioned, and other factors.

In certain aspects, an array of electrodes (e.g., an ECoG array, microelectrode array, EEG array) is positioned on the surface of the speech motor cortex such that the array covers the entire or substantially the entire region of the speech motor cortex corresponding to the somatotopic arrangement of articulatory kinematic representations of the subject. For example, the electrode array may be disposed on the surface of the speech motor cortex from −100 mm to +100 mm, from −80 mm to +80 mm, from −60 mm to +60 mm, from −40 mm to +40 mm, or from −20 mm to +20 mm relative to the central sulcus along the anterior-posterior axis. Alternatively, or additionally, the electrode array may be disposed on the surface of the speech motor cortex from a location at or proximal to the Sylvian fissure to a distance of 500 mm or less, 400 min or less, 300 mm or less, 200 mm or less, 100 mm or less, 90 mm or less, 80 mm or less, 70 mm or less, 60 mm or less, 50 mm or less, or 40 mm or less from the Sylvian fissure along the dorsal-ventral axis. Non-limiting examples of an array and example positioning thereof can be found in U.S. Pat. No. 9,905,239, which is hereby incorporated by reference in its entirety.

In certain embodiments, a ground electrode or reference electrode may be positioned. A ground or reference electrode may be placed at any convenient location, where such locations are known to those of skill in the art. In certain embodiments, a ground electrode or reference electrode is a scalp electrode. A scalp electrode may be placed on a subject's forehead or in any other convenient location.

Aspects of the present disclosure comprise detecting a plurality of signals when an individual is intended to produce a speech output. In some embodiments, the plurality of signals are acquired by any known neurophysiological recording device. In some embodiments, the plurality of signals are acquired through optical devices. Optical devices that can be used to acquire the plurality of signals include, but are not limited to: instrinic optical signal (I0S) imaging, extrinsic optical signal (EOS) imaging, Doppler flowmetry (LDF), near-infrared (NIR) spectrometer, functional optical coherence tomography (fOCT), and surface plasmon resonance (SPR). Other techniques such as radioactive imaging can be used to acquire the plurality of signals. Non-limiting examples include radioactive imaging of changes in blood flow, magnetoencephalography (MEG), thermal imaging, positron-emission tomography (PET), functional magnetic resonance imaging (fMRI), and diffuse optical tomography (DOT). In some embodiments, the plurality of signals are acquired by microelectrodes. In some embodiments, the plurality of signals are acquired by ECoG. In some embodiments, the plurality of signals are acquired by EEG. In some embodiments, the plurality of signals are acquired by intracranial spike recordings. In some embodiments, the plurality of signals are neural signals. In some embodiments, the plurality of signals comprise local field potentials from the speech motor cortex of the brain. In some embodiments, the plurality of signals are acquired by functional magnetic resonance imaging (fMRI), blood oxygen level-dependent (BOLD)-fMRI, diffusion tensor imaging (DTI), manganese-enhanced MRI (ME-MRI), multiphoton microscopy (MP), magnetoencephalographic imaging (MEGI), and the like.

In some embodiments, the method comprises extracting speech-related features from the neural signals. In some embodiments, extracting speech-related features from the neural signals comprises filtering the plurality of signals in a high gamma frequency range to obtain neural signals in the auditory and sensorimotor brain regions. In some embodiments, plurality of signals are obtained from auditory and sensorimotor brain regions selected from the vSMC, STG, and IFG. In some embodiments, the plurality of signals comprise the high-gamma frequency component of the local field potentials. The high-gamma frequency component of the local field potential is a high-gamma frequency range of the plurality of signals associated with an intended speech output. In some embodiments, the high-gamma frequency range ranges from 70-200 Hz (e.g. 70-75 Hz, 75-80 Hz, 80-85 Hz, 95-90 Hz, 90-95 Hz, 95-100 Hz, 100-105 Hz, 105-110 Hz, 110-115 Hz, 115-120 Hz, 120-125 Hz, 125-130 Hz, 130-135 Hz, 135-140 Hz, 140-145 Hz, 145-150 Hz, 150-155 Hz, 155-160 Hz, 160-165 Hz, 165-170 Hz, 170-175 Hz, 175-180 Hz, 180-185 Hz, 185-190 Hz, 190-195 Hz, or 195-200 Hz). In some embodiments, the high-gamma frequency range ranges from 70-150 Hz. In some embodiments, the analytic amplitude of the high-gamma frequency component of the local field potentials was extracted with the Hilbert transform and down-sampled to 200 Hz. In some embodiments, the plurality of signals comprise a low frequency component (e.g. 1-30 Hz) extracted with a 5th order Butterworth bandpass filter and parallelly aligned with the high- gamma amplitude.

In some embodiments, electrodes for which neural signals are collected are from electrodes located on cortical areas related to speech, such as the vSMC, STG, and/or IFG.

In some embodiments, the one or more speech related features comprises the high-gamma amplitude frequency range that correlated with multi-unit firing rates within the neural signals. In some embodiments, the high gamma amplitude frequency range comprises the temporal resolution to resolve fine articulatory movements in the individual.

In some embodiments, the method further comprises recording acoustic signals (e.g. audio signals). In some embodiments, the method further comprises translating the recorded acoustic signals into phonetic transcriptions or text. In some embodiments, the method comprises aligning the time of the acoustic signals with one or more external context-related ues and/or speech events. In some embodiments, recording acoustic signals occurs during one or more external context-related cues. In some embodiments, the acoustic signals are recorded as acoustic waveforms. In some embodiments, the acoustic signals are represented as spectral features with the following parameters: a 25 mel-frequency cepstral coefficients (MFCCs), and/or 5 sub-band voicing strengths for glottal excitation modelling, pitch, and voicing (e.g. 32 features). In some embodiments, the acoustic parameters are configured to emphasize perceptually relevant acoustic features while maximizing audio reconstruction quality.

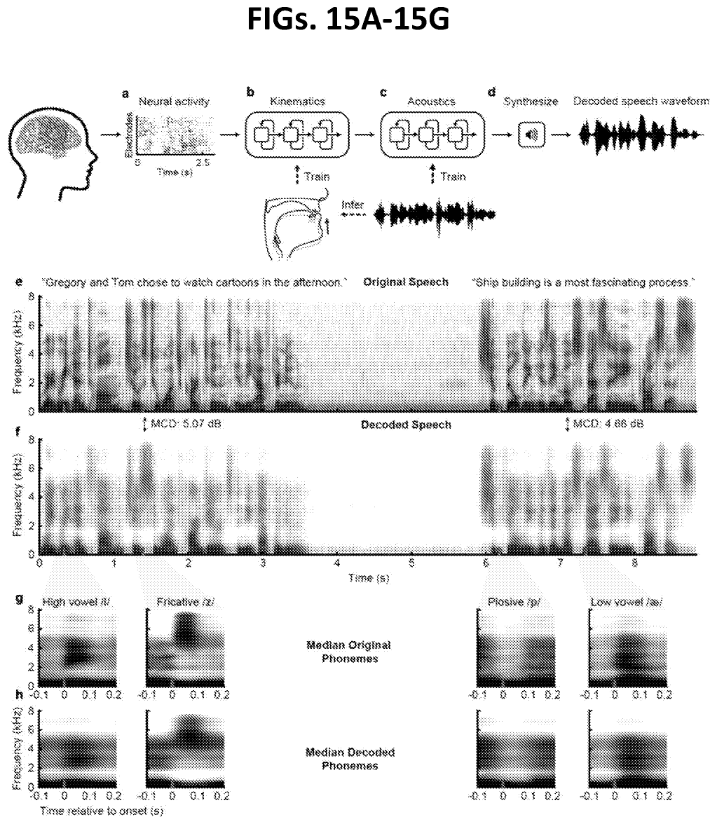

In some embodiments, the method further comprises one or more processors. In some embodiments, the one or more processors comprises one or more decoders. In some embodiments, the one or more decoders is configured to decode and/or synthesize neural signals. In some embodiments, the one or more decoders is configured to decode and/or synthesize acoustic signals. In some embodiments, the one or more decoders are configured to synthesize the neural signals into acoustic signals. In some embodiments, neural signals and acoustic signals are recorded simultaneously. In some embodiments, neural signals and acoustic signals are recorded simultaneously during one or more external context-related cues. In some embodiments, the method further comprises assessing and/or computing the spectral distortion between the recorded acoustic signals and the decoded acoustic signals synthesized from the neural signals. In some embodiments, the spectral distortion is computed using a Mel-cepstral distortion (MCD) metric (e.g. as shown in FIG. 15E-15f). The use of Mel-frequency bands as an acoustic parameter emphasizes the distortion of perceptually relevant frequency bands of the audio spectrogram.

In some embodiments, MCD of the synthesized speech is calculated when compared to original ground-truth audio recordings (e.g. recorded acoustic signals). MCD is an objective measure of error determined from MFCCs and is correlated to subjective perceptual judgments of acoustic quality. For reference acoustic features mc^{(y)} and decoded features mc^{(ŷ)},

$$M C D = \frac{10}{ln ( 10 )} \sqrt{{\sum }_{0 < d < 25} {( m {c}_{d}^{( y )} - m {c}_{d}^{( \hat{y} )} )}^{2}} \qquad ( 1 )$$

In some embodiments, the method comprises quantifying one or more external context-related cues. In some embodiments, the one or more external context-related cues comprises silent speech. In some embodiments, the method comprises decoding silent speech. In some embodiments, the method comprises assessing decoding performance by decoding silent speech compared to the audible speech of a word, sentence, and/or paragraph uttered immediately prior to silent speech. In some embodiments, the method comprises dynamically time warping the decoded silent speech MFCCs to the MFCCs of the audible condition and computing Pearson' s correlation coefficient and Mel-cepstral distortion.

In some embodiments, the method comprises detecting when the individual is intended to produce a speech output. In some embodiments, said detecting comprises recording neural signals during one or more external context-related cues. In some embodiments, said detecting comprises extracting high-gamma amplitude signals and/or low frequency signals from the raw neural signals of each electrode.

In some embodiments, the method comprises extracting speech-related features from the signals and decoding the intended speech output in real-time.

In some embodiments, the method further comprises timing the individual during the speech event. In some embodiments, the method further comprises timing the individual during the one or more external context-related cues. In some embodiments, the decoder synthesizes one or more external context-related cues based on the kinematic movements (e.g. articulatory kinematics) of the individual during a speech event and/or one or more external context-related cues. In some embodiments, the articulatory kinematics are configured to capture the physiological process by which speech is generated and/or encoded in the speech motor cortex (e.g. vSMC). In some embodiments where the one or more external context- related cues comprises silent mimes, the decoder synthesizes silent mimed speech based on the kinematic movements of the individual during the silent mimes. In some embodiments, the decoder synthesizes spectral features of silently mimed speech that are never audibly uttered. In some embodiments, the silently mimed speech is dynamically time-warped according to spectral features of the acoustic signals.

In some embodiments, the method further comprises translating the speech events into phonetic transcriptions or text. In some embodiments, the method comprises comparing median spectrograms of phonemes from original (e.g. recorded acoustic signals) and decoded (e.g. acoustic signals decoded from neural signals) audio. In some embodiments, the acoustic signals decoded from neural signals closely resemble original speech. In some embodiments, the method further comprises computing phone likelihoods at each time point during the speech event.

In some embodiments, decoding comprises predicting time segments of the neural signals that that are associated with speech events. In some embodiments, the time segment comprises at least 30 seconds, at least 1 minute, at least 5 minutes, at least 10 minutes, at least 20 minutes, at least 25 minutes, at least 30 minutes, at least 35 minutes, at least 40 minutes, at least 50 minutes, at least 55 minutes, or at least 60 minutes of speech.

In some embodiments, decoding the intended speech output comprises machine learning algorithms that identify spatiotemporal neural patterns associated with the speech events. In some embodiments, the machine learning algorithms require speech training data associated with a speech event. In some embodiments, the machine learning algorithm require at least 30 seconds, at least 1 minute, at least 5 minutes, at least 10 minutes, at least 20 minutes, at least 25 minutes, at least 30 minutes, at least 35 minutes, at least 40 minutes, at least 50 minutes, at least 55 minutes, or at least 60 minutes of speech training data. In some embodiments, the spatiotemporal neural patterns comprise rapid evoked responses in the STG during the speech events. In some embodiments, decoding the intended speech output comprises predicting the temporal onsets and offsets of the speech events based on the rapid evoked responses in the STG.

In some embodiments, wherein the method further comprises displaying the decoded speech output. In some embodiments, the speech output is displayed on a screen. In some embodiments, the speech output is displayed on a screen as one or more syllables, words, parts of words, phrases, utterances, paragraphs, sentences, and/or a combination thereof. In some embodiments, the speech output is displayed on a screen as one or more sentences. In some embodiments, the speech output is displayed on a computer, a tablet computer or smart phone, or any related computing device. In some embodiments, the tablet computer or smartphone runs an operating system selected from an iOS™ operating system, an Android™ operating system, a Windows^{TM} operating system, or any other tablet- or smartphone-compatible operating system.

Aspects of the present disclosure include a non-transitory computer readable medium storing instructions that, when executed by one or more processors and/or computing devices, cause the one or more processors and/or computing devices to perform the steps for decoding speech events in an individual, as provided herein.
1. Aspects of the present disclosure include a non-transitory computer readable medium storing instructions that, when executed by one or more processors and/or computing devices, cause the one or more processors and/or computing devices to perform the steps for decoding auditory perceived speech or verbal produced speech in an individual, as provided herein.

In some embodiments, the method of the present disclosure method is carried out using a receiver unit, comprising: a wireless receiver in communication with a wireless transmitter that receives the plurality of signals detected from at least three electrodes; one or more processors; a non-transient computer-readable medium comprising instructions that, when executed by the one or more processors, cause the one or more processors to: perform one or more filters on the plurality of signals; decode the plurality of signals into articulatory movement representations; and output the plurality of acoustic signals into a speech output.

In some embodiments, the method comprises filtering the plurality of signals with one or more filters. In some embodiments, the one or more filters comprises one or more notch filters. In some embodiments, the one or more filters comprises one or more band-pass finish impulse response (FIR) filters. In some embodiments, the one or more processors is configured to extract analytic amplitude values across the one or more band-pass FIR filters applied to the plurality of signals. In some embodiments, the one or more processors is configured to average the analytic amplitude values across the one or more band-pass FIR filters to obtain one or more high gamma analytic amplitude signals (e.g. high gamma frequency range signals).

In some embodiments, the one or more processors is configured to normalize and store the one or more high gamma analytic amplitude signals in an event detector process. In some embodiments, the event detector process is configured to analyze the high gamma analytic signals. In some embodiments, the gamma analytic signals are analyzed at one or more time points to predict the onset and offset of auditory perceived or verbal produced speech events. In some embodiments, the one or more time points comprises 10 or more ms time points, 20 or more ms time points, 30 or more ms time points, 40 or more ms time points, or 50 or more ms time points. In some embodiments, the one or more time points comprises 10 or more ms time points, 50 or more ms timepoints, 100 or more ms time points, 150 or more ms time ponts, 200 or more time points, 250 or more ms timepoints, 300 or more ms time points, 350 or more ms time points, 400 ms or more time points, 450 or more ms time points, or 500 or more ms time points.

In some embodiments, the one or more processors are configured to decode the one or more high gamma analytic amplitude signals into the speech output.

In some embodiments, the one or more processors is a neural decoder. In some embodiments, the method comprises two or more processors, three or more processors, four or more processors, or five or more processors. In some embodiments, the one or more processors comprises a neural decoder comprising a bidirectional long short-term memory comprising an algorithm for decoding the plurality of acoustic signals into the speech output. In some embodiments, the one or more processors is one or more (e.g. two or more, three or more, four or more, or five or more) stacked 3-layer bidirectional long short term memory (bLSTM) recurrent neural networks. In some embodiments, a first stacked 3-layer bLSTM is configured to learn the mapping between time point windows (e.g. 300 ms windows) of high- gamma and local field potential signals and the corresponding single time point of 32 articulatory features related to movement of the vocal tract. In some embodiments, a second stacked 3-layer bLSTM is configured to learn the mapping between the output of decoded articulatory features and 32 acoustic parameters for decoding an intended speech output (e.g. one or more syllables, words, parts of words, phrases, utterances, paragraphs, sentences, and/or a combination thereof). In some embodiments, the first and/or second stacked 3-layer bLSTM is trained with a learning rate of 0.001.

In some embodiments, the bLSTM decodes speech-related features from the neural signals. In some embodiments, the speech-related features are articulatory kinematic features from the neural or optical signals. In some embodiments, the speech-related features comprises articulatory movement representations. In some embodiments, the one or more processors decodes the articulatory movement representations into acoustic signals. In some embodiments, the speech-related features comprises articulatory kinematic features. In some embodiments, the one or more processors decodes the articulatory kinematic features into acoustic signals. In some embodiments, the one or more processors decodes the articulatory movement representations and the articulatory kinematic features into acoustic signals. In some embodiments, a second bLSTM decodes acoustic features from the speech-related features of the neural or optical signals. In some embodiments, the bLSTM decodes acoustic features from the decoded articulatory kinematic features from the neural signals. In some embodiments, the bLSTM decodes acoustic features from the articulatory movement features. In some embodiments, the articulatory movement features comprise recorded acoustic signals during a speech event.

In some embodiments, the one or more processors comprises an algorithm for decoding an intended speech output. In some embodiments, the algorithm is an articulatory kinematics inference model. In some embodiments, the articulatory inference model comprises a stacked deep encoder-decoder. In some embodiments, the encoder combines phonological and acoustic representations into a latent articulatory representation that is then decoded to reconstruct the original acoustic signal during a speech event. In some embodiments, the latent representation is initialized with inferred articulatory movement from Electromagnetic Midsagittal Articulography (EMA) and appropriate manner features.

In some embodiments, the one or more processors comprises a machine learning algorithm for estimating 32 dimensional articulatory kinematic trajectories (e.g. acoustically consequential movements of the vocal tract) using only produced acoustic and phonetic transcriptions or text. Dimensional articulatory kinematic trajectories are described in Chartier et al. (Neuron (2018) 98:5, pgs 1042-1054), which is hereby incorporated by reference in its entirety. In some embodiments, the dimensional articulatory kinematic trajectories are represented as place manner tuples (representations as continuous binary valued features) that incorporate physiological aspects in EMA, which include one or more of the tongue blade, tongue tip, jaw, upper lip, lower lip, velar stop, velar nasal, palatal approximant, palatal fricative, palatal affricate, labial stop, labial approximant, labial nasal, glottal fricative, dental fricative, labiodental fricative, alveolar stop, alveolar approximant, alveolar nasal, alveolar lateral, alveolar fricative, unconstructed, and voicing. In some embodiments, the machine learning algorithm comprises an existing annotated speech database (Wall Street Journal Corpus) and trained speaker independent deep recurrent network regression models to predict the place-manner tuple vectors from the acoustic signal of a speech event.

In some embodiments, the one or more processors comprises an autoencoder. In some embodiments, the autoencoder is a recurrent neural network encoder that is trained to convert phonological and acoustic features to the initialized 32 articulatory representations. In some embodiments, the one or more processor comprises a decoder, wherein the decoder converts the articulatory representation back to acoustic signals. In some embodiments, the one or more processors (e.g. stacked neural network) is re-trained optimizing the joint loss on acoustic and EMA parameters. After convergence, the encoder is used to estimate the final articulatory kinematic features that act as the intermediate to decode acoustics from neural signals.

In some embodiments, the one or more processors further comprises an autoencoder configured to convert phonological and acoustic features of the audible speech or silent speech acoustic signals into one or more articulatory representations. In some embodiments, the one or more processors further comprises a decoder configured to convert the one or more articulatory representations to audible speech or silent speech acoustic signals. In some embodiments, the one or more processors further comprises an encoder configured to estimate final articulatory kinematic features, wherein the final articulatory kinematic features are used in an algorithm to decode articulatory movement features from the neural signals.

In some embodiments, the one or more processors comprises a deep neural network comprising an algorithm for decoding the audible speech or silent speech acoustic signal as mel frequency cepstral coefficients. In some embodiments, the deep neural network comprises an algorithm for decoding the audible speech or silent speech acoustic signals as 25 dimensional mel frequency cepstral coefficients.

In some embodiments, the one or more processors comprises a hidden Markov model based acoustic model configured to perform sub-phonetic alignment.

In some embodiments, the one or more processors comprises a Kullback-Leibler

(KL) divergence model configured to compare the distribution of a decoded phoneme of the neural signals to a distribution of a ground-truth phoneme.

Aspects of the present disclosure further include methods of decoding auditory perceived speech or verbal produced speech in an individual, the method comprising: contacting an electrode array with the cortical region of the brain in the individual; conducting speech perception training on the individual, wherein speech perception training comprises listening to pre-recorded questions; conducting speech production training on the individual, wherein speech production training comprises reading one or more answers on a screen; conducting speech testing on the individual, wherein speech testing comprises listening to pre- recorded questions and responding verbally with answers to the pre-recorded questions; recording a time-aligned audio of the speech perception training, speech production training, and speech testing on the individual; recording neural signals; analyzing the neural signals in the cortical region of the brain; and decoding the neural signals into a speech output.

In some embodiments, the method further comprises translating the time-aligned audio into phonetic transcriptions.

In some embodiments, the method further comprises determining time points at which the recorded neural signals is associated with speech perception, speech production, speech testing, or silence.

In some embodiments, the method further comprises determining which electrodes in the electrode array are responsive to the speech perception training, speech production training, or speech testing.

In some embodiments, the electrode array comprises 3 or more electrodes.

In some embodiments, decoding comprises computing speech perception, speech production, or silence probabilities. In some embodiments, the decoding is computed with one or more processors as described in the present disclosure. In some embodiments, the method comprises a non-transient computer-readable medium comprising instructions that, when executed by the one or more processors, cause the one or more processors to perform its intended function as disclosed herein.

In some embodiments, the methods of the present disclosure include methosd of decoding intended speech events in an individual, the method comprising extracting speech-related features from a plurality of signals from the brain of the individual when the individual is intended to produce a speech output; and decoding, with one or more decoding constraints, the intended speech output from the plurality of signals.

In some embodiments, the plurality of signals comprises neural signals acquired by electrocorticography (ECoG), electroencephalography (EEG), or microelectrodes.

In some embodiments, the plurality of signals comprises optical signals, wherein the optical signals are fast optical signals (FOS) or event-related optical signals (EROS) or BOLD signals in functional magnetic resonance imaging (fMRI).

In some embodiments, said acquiring comprises contacting at least three electrodes that detect the plurality of signals with at least one region of the brain. In some embodiments, the at least one region of the brain comprises the speech motor cortex of the brain. In some embodiments, the at least one region of the brain is selected from the sensorimotor cortex (SMC), superior temporal gyms (STG), and inferior frontal gyms (IFG).

In some embodiments, contacting comprises implantation on the surface of the speech motor cortex of the brain. In some embodiments, the plurality of signals comprise local field or action potentials from the at least one region of the brain. In some embodiments, the plurality of signals comprise the high-gamma frequency or other frequency components of the local field potentials.

In some embodiments, the method further comprises detecting when the individual is intended to produce a speech output.

In some embodiments, wherein extracting speech-related features from the signals and decoding the intended speech output occurs in real-time. In some embodiments, where the one or more external context-related cues comprises listening to pre-recorded questions. In some embodiments, the one or more external context-related cues comprises reading one or more answers on a screen. In some embodiments, wherein the one or more external context-related cues comprises responding to pre-recorded questions. In some embodiments, wherein responding to pre-recorded questions comprises a verbal response. In some embodiments, wherein the verbal response is a sound. In some embodiments, wherein the sound is selected from the group consisting of: a phoneme, formant acoustics of a vowel, a diphone, a triphone, a consonant-vowel transition, a syllable, a word, a phrase, a sentence, and combinations thereof. In some embodiments, wherein the one or more external context-related cues comprises visually responding to pre-recorded questions.

In some embodiments, wherein the method further comprises timing the individual during the speech event. In some embodiments, wherein the one or more external context-related cues comprises silently mimed speech.

In some embodiments, wherein the method further comprises translating the speech events into phonetic transcriptions or text. In some embodiments, wherein the method further comprises computing phone likelihoods at each time point during the speech event.

In some embodiments, wherein said extracting speech-related features comprises filtering the plurality of signals in a high gamma frequency range to obtain neural signals in the auditory and sensorimotor brain regions. In some embodiments, wherein the plurality of signals are obtained from auditory and sensorimotor brain regions selected from the vSMC, STG, and IFG.

In some embodiments, wherein the high gamma frequency ranges from 70 to 200

Hz.

In some embodiments, wherein decoding comprises predicting time segments of the neural signals that that are associated with speech events. In some embodiments, wherein the intended speech output is decoded before the produced speech output.

In some embodiments, wherein the neural signals comprise rapid evoked responses in the one or more regions in the brain during the speech events.

In some embodiments, wherein decoding comprises predicting the temporal onsets and offsets of the speech events based on the rapid evoked responses in the one or more regions of the brain.

In some embodiments, wherein the method further comprises displaying the decoded speech output. In some embodiments, wherein the speech output is displayed on a screen as one or more words. In some embodiments, wherein the speech output is displayed on a screen as one or more sentences.

In some embodiments, wherein the method is carried out using a receiver unit, comprising: a receiver (e.g. wireless or non-wireless) in communication with a transmitter that receives the plurality of signals detected from the at least three electrodes; one or more processors; a non-transient computer-readable medium comprising instructions that, when executed by the one or more processors, cause the one or more processors to: perform one or more filters on the plurality of signals; decode the plurality of signals into articulatory movement representations; and output the plurality of acoustic signals into a speech output.

In some embodiments, wherein the one or more processors is a neural decoder. In some embodiments, wherein the one or more processors decodes the articulatory movement representations into acoustic signals. In some embodiments, wherein the one or more filters comprises one or more notch filters. In some embodiments, wherein the one or more processors is further configured to stream the plurality of signals onto a computer. In some embodiments, wherein the one or more filters comprises one or more band-pass finish impulse response (FIR) filters. In some embodiments, wherein the one or more processors is configured to extract analytic amplitude values across the one or more band-pass FIR filters applied to the plurality of signals. In some embodiments, wherein the one or more processors is configured to average the analytic amplitude values across the one or more band-pass FIR filters to obtain one or more gamma (e.g. high) analytic amplitude signals. In some embodiments, wherein the one or more processors is configured to normalize and store the one or more gamma (e.g. high) analytic amplitude signals. In some embodiments, wherein the one or more processors comprises an event detector process configured to analyze the gamma (e.g. high) analytic signals.

In some embodiments, wherein the gamma (e.g. high) analytic signals are analyzed at one or more time points to predict the onset and offset of auditory perceived or verbal produced speech events. In some embodiments, wherein the one or more processors are configured to decode the one or more high gamma analytic amplitude signals into the speech output.

In some embodiments, wherein the neural decoder comprises a bidirectional long short-term memory recurrent neural network comprising an algorithm for decoding the plurality of acoustic signals into the speech output.

Aspects of the present disclosure include a method of decoding auditory perceived speech or verbal produced speech in an individual, the method comprising: a) contacting an electrode array with the cortical region of the brain in the individual; b) conducting at least one of: speech perception training on the individual, wherein speech perception training comprises listening to a sound; speech production training on the individual, wherein speech production training comprises reading; speech testing on the individual, wherein speech testing comprises listening to a sound and responding verbally to the sound; e) recording a time-aligned audio of the speech perception training, speech production training, and speech testing on the individual; f) recording a plurality of signals in step b); g) analyzing the neural signals in the cortical region of the brain; and i) decoding the neural signals into a speech output.

In some embodiments, wherein the plurality of signals are neural signals. In some embodiments, wherein the method further comprises translating the time-aligned audio in step e) into phonetic transcriptions or text. In some embodiments, wherein the method further comprises determining time points at which the recorded neural signals is associated with speech perception, speech production, speech testing, or silence.

In some embodiments, the method further comprises determining which electrodes in the electrode array are responsive to the speech perception training, speech production training, or speech testing. In some embodiments, the electrode array comprises three or more electrodes. In some embodiments, decoding comprises computing speech perception, speech production, or silence probabilities.

### SYSTEMS—ENCODING AND DECODING SPEECH USING ARTICULATORY PHYSIOLOGY

Also provided are systems for performing the methods of the present disclosure. Such systems include speech communication systems that output speech based on a speech pattern signal in response to physiological feature signals according to the present disclosure. In some embodiments, the system comprises a processor comprising memory operably coupled to the processor, wherein the memory includes instructions stored thereon, which when executed by the processor, cause the processor to perform one or more of the steps of the methods of the present disclosure.

Aspects of the present disclosure include a non-transitory computer readable medium storing instructions that, when executed by one or more processors and/or computing devices, cause the one or more processors and/or computing devices to perform the steps for generating a speech output, as provided herein.

In some embodiments, the system comprises a processor comprising memory operably coupled to the processor, wherein the memory includes instructions stored thereon, which when executed by the processor, cause the processor to receive a physiological feature signal associated with a spatiotemporal movement of a vocal tract articulator. In some embodiments, the system comprises a processor comprising memory operably coupled to the processor, wherein the memory includes instructions stored thereon, which when executed by the processor, cause the processor to generate a speech pattern signal in response to the physiological feature signal. In some embodiments, the system comprises an output for putting speech that is based on the speech pattern signal.

In some embodiments, the system comprises a processor comprising memory operably coupled to the processor, wherein the memory includes instructions stored thereon, which when executed by the processor, cause the processor to receive a physiological feature signal associated with a spatiotemporal movement of a vocal tract articulator; generate a speech pattern signal in response to the physiological feature signal; and an output for outputting speech that is based on the speech pattern signal.

In some embodiments, the processor is one or more processors. In some embodiments, the method comprises two or more processors, three or more processors, four or more processors, or five or more processors.

In some embodiments, the one or more processors comprises bidirectional long-short term memory (bLSTM). In some embodiments, the bidirectional long-short term memory comprises algorithm for encoding the physiological feature signal. In some embodiment, the bLSTM comprises an algorithm for decoding the physiological feature signal to a speech pattern signal. In some embodiments, the bLSTM comprises an algorithm for decoding physiological signal to auditory speech. In some embodiments, the bLSM comprises an algorithm for decoding physiological signal to text.

In some embodiments, the one or more processors comprises a bidirectional long-short term memory comprising an algorithm for decoding speech pattern signals in response to the physiological feature signals, linguistic signals, and/or acoustic signals into an output for outputting speech that is based on the speech pattern signals, linguistic signals, and/or acoustic signals associated with a physiological feature. In some embodiments, the one or more processors is one or more (e.g. two or more, three or more, four or more, or five or more) stacked 3-layer bLSTM recurrent neural networks. In some embodiments, a first stacked 3-layer bLSTM is configured to learn the mapping between time point windows (e.g. 300 ms windows) of high-gamma and local field potential signals (e.g. one or more brain signals) and the corresponding single time point of 32 vocal tract articulators related to movement of the vocal tract. In some embodiments, a second stacked 3-layer bLSTM is configured to learn the mapping between the speech output of vocal tract articulators and 32 acoustic parameters for outputting auditory speech or text (e.g. one or more sounds or text of syllables, words, parts of words, phrases, utterances, paragraphs, sentences, and/or a combination thereof). In some embodiments, the first and/or second stacked 3-layer bLSTM is trained with a learning rate of 0.001.

In some embodiments, the bLSTM generates a speech pattern signal in response to the physiological feature signal. In some embodiments, the one or more processors encodes the brain signals associated with one or more spatiotemporal movements of a vocal tract articulator to generate a physiological feature signal. In some embodiments, the one or more processors encodes the physiological feature signal into a speech output. In some embodiments, a second bLSTM encodes acoustic signals. In some embodiments, the bLSTM encodes acoustic features from the physiological features. In some embodiments, the bLSTM encodes phonological and acoustic signals into a physiological feature signal. In some embodiments, the physiological feature signal is decoded into a speech pattern signal. In some embodiments, the bLSTM decodes the physiological feature signal to auditory speech.

In some embodiments, wherein the processor comprises a deep neural network (DNN) In some embodiments, the deep neural network comprises algorithm for decoding the physiological feature signal to a speech pattern signal. In some embodiments, the deep neural network comprises algorithm for decoding physiological signal to auditory speech. In some embodiments, the deep neural network comprises algorithm for decoding physiological signal to text.

In some embodiments, the system comprises a BLSTM and a deep neural network (DNN).

In some embodiments, the deep neural network comprises algorithm for decoding physiological signal as mel frequency cepstral coefficients (MFCC). The use of Mel-frequency bands as an acoustic parameter emphasizes the distortion of perceptually relevant frequency bands of the audio spectrogram.

In some embodiments, the deep neural network comprises algorithm for decoding physiological signal as 25 dimensional mel frequency cepstral coefficients.

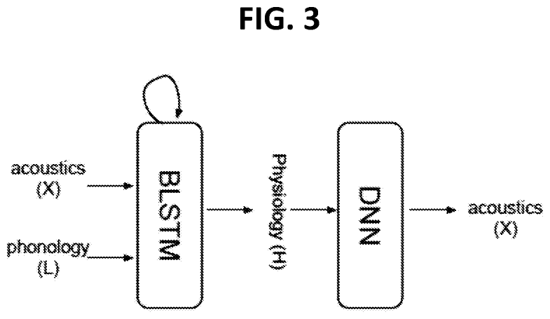

In some embodiments, the bLSTM and/or the deep neural network comprises a encoder-decoder network. In some embodiments, the bLSTM and/or deep neural network is configured to encode a physiological feature, a phonological feature, and/or a acoustic features. In some embodiments, the bLSTM and/or deep neural network encodes a physiological feature, a phonological feature, and/or an acoustic features into a 31 dimensional feature space. In some embodiments, the bLSTM and/or deep neural network encodes a physiological feature, a phonological feature, and/or a acoustic features into a 32 dimensional feature space. In some embodiments, the encoder network is a recurrent network. In some embodiments, a sequence-to-sequence regression was used with bidirectional LSTM cells to encode the physiological layer. In some embodiments, a decoder is configured to be trained to decode from the physiological feature signals to acoustic feature signals, coded as 25 dimensional mel frequency cepstral coefficients. In some embodiments, the decoder comprises a feedforward network. In some embodiments, the encoder network and the decoder network are trained individually. In some embodiments, the one or more processors are stacked together and backpropagated through the whole training data as a single network as illustrated in FIG. 3.

In some embodiments, the processor is configured to provide a mean squared error on the acoustic signal, and an auxiliary loss function. In some embodiments, the mean squared error on the EMA displacement traces in the bottleneck layer for which there is groundtruth data. In some embodiments, the augmented manner features are not included in the cost function allowing the network to freely change them through the backpropagation training.

In some embodiments, the physiological feature signal comprises a dataset associated with spatiotemporal movement of one or more vocal tract articulators. In some embodiments, the vocal tract articulator is selected from the group consisting of the upper lip, lower lip, lower incisor, tongue tip, tongue blade, tongue dorsum and larynx. In some embodiments, the dataset comprises measurements of the caudo-rostral displacements of the one or more of the vocal tract articulators. In some embodiments, the physiological feature comprises a electromagnetic midsagittal articulography dataset associated with spatiotemporal movement of one or more vocal tract articulators.

In some embodiments, the system comprises memory operably coupled to the processor wherein the memory includes instructions stored thereon, which when executed by the processor, cause the processor to: receive one or more signals from the brain; and associate the brain signals to one or more spatiotemporal movements of a vocal tract articulator to generate a physiological feature signal; and generate a speech pattern signal in response to the physiological feature signal.

In some embodiments, the system comprises electrical leads (e.g. electrodes and/or electrode arrays) for receiving signals from all or a part of the ventral sensorimotor cortex of the brain. In some embodiments, wherein the output is configured to output auditory speech or text. In some embodiments, the output is an audio speaker. In some embodiments, output is a text generator. In some embodiments, output is a speech generator.

Aspects of the present disclosure include a system comprising input for receiving one or more of: a linguistic signal; an acoustic signal; and a processor comprising memory operably coupled to the processor wherein the memory includes instructions stored thereon, which when executed by the processor, cause the processor to: associate a physiological feature with an inputted linguistic or acoustic signal; and an output configured to output a speech signal in response to the physiological feature.

In some embodiments, the processor is one or more processors. In some embodiments, the processor comprises bidirectional long-short term memory. In some embodiments, the bidirectional long-short term memory comprises algorithm for encoding the physiological signal associated with the inputted linguistic or acoustic signal.

In some embodiments, the processor comprises a deep neural network (DNN). In some embodiments, the deep neural network comprises algorithm for decoding physiological signal to a speech signal. In some embodiments, the deep neural network comprises algorithm for decoding physiological signal to auditory speech. In some embodiments, the deep neural network comprises algorithm for decoding physiological signal to text. In some embodiments, the deep neural network comprises algorithm for decoding physiological signal as mel frequency cepstral coefficients. In some embodiments, the deep neural network comprises algorithm for decoding physiological signal as 25 dimensional mel frequency cepstral coefficients.

In some embodiments, the physiological feature comprises a dataset associated with spatiotemporal movement of one or more vocal tract articulators.

In some embodiments, the vocal tract articulator is selected from the group consisting of the upper lip, lower lip, lower incisor, tongue tip, tongue blade, tongue dorsum and larynx.

In some embodiments, the dataset comprises measurements of the caudo-rostral displacements of the one or more of the vocal tract articulators.

In some embodiments, the physiological feature comprises a electromagnetic midsagittal articulography dataset associated with spatiotemporal movement of one or more vocal tract articulators.

In some embodiments, the system comprises memory operably coupled to the processor wherein the memory includes instructions stored thereon, which when executed by the processor, cause the processor to: receive one or more signals from the brain; and associate the brain signals to one or more spatiotemporal movements of a vocal tract articulator to generate a physiological feature signal; and generate a speech pattern signal in response to the physiological feature signal.

In some embodiments, the system further comprises electrical leads (e.g. electrodes and/or electrode arrays) for receiving signals from all or a part of the ventral sensorimotor cortex of the brain.

In some embodiments, the output is configured to output auditory speech or text. In some embodiments, the output is an audio speaker. In some embodiments, the output is a text generator. In some embodiments, the output is a speech generator.

In some embodiments, the systems of the present disclosure comprise a neurotransmitter that detects brain signals associated with one or more spatiotemporal movements of a vocal tract articulator when operably coupled to the speech motor cortex of a subject while the subject imagines producing a speech sound, and a transmitter (e.g., a wireless transmitter) that transmits the detected speech production signals. The receiver unit includes: a receiver (e.g., a wireless receiver) in communication with the transmitter that receives the detected speech production signals; a speech generator, a processor and a memory (e.g., a non-transitory computer readable medium) that includes instructions which, when executed by the processor, derive a speech signal pattern from the detected speech production signals, correlates the speech production signal pattern with a reference speech signal pattern to decode the speech sound, and communicates the speech sound using the speech generator.

As set forth above, systems of the present disclosure include a neurosensor which includes a transmitter that transmits the detected speech signal patterns. In certain aspects, the transmitter is a wireless transmitter. Wireless transmitters of interest include, but are not limited to, WiFi-based transmitters, Bluetooth-based transmitters, radio frequency (RF)-based transmitters, and the like. The wireless receiver of the receiver unit is selected such that it is compatible with the wireless format of the wireless transmitter.

In some embodiments, systems of the present disclosure include a speech generator. In certain aspects, the speech generator comprises a speaker that produces the speech sound in audible form. For example, the speaker may produce the speech sound in a manner that replicates a human voice. Alternatively, or additionally, the speech generator may include a display that displays the speech sound in text format. According to certain embodiments, the speech generator includes both a speaker that produces the speech sound in audible form and a display that displays the speech sound in text format. In certain aspects, the receiver unit includes a control that enables the subject to toggle between producing the speech sound in audible form, displaying the speech sound in text format, and both. The speech generator is capable of generating any of the speech sounds actually or imaginarily produced by the subject, e.g., a phoneme, a diphone, a triphone, a syllable, a consonant-vowel transition (CV), a word, a phrase, a sentence, or any combination of such speech sounds. In certain aspects, the speech generator is capable of generating the formants and/or pitch of the speech sound(s) actually or imaginarily produced by the subject, e.g., based on information relating to formants and pitch encoded in the speech production signals and patterns thereof. For example, speech production signal patterns which include information relating to formants and pitch may be correlated to reference speech production signal patterns associated with known formants and pitches (e.g., as established during a training period), and the speech generator may produce the speech sound (e.g., in audible form or text format) with the correlated formants and pitch. Inclusion of the formants and pitch in the speech sound produced by the speech generator is useful, e.g., to make the speech sound more natural and/or understandable to those with whom the subject is communicating.

In certain aspects, the receiver unit is a unit dedicated solely to receiving and processing speech production signals detected by the neurosensor, deriving and decoding speech production signal patterns, and the like. In other aspects, the receiver unit is a device commonly used among the subject's population which is capable of performing the functions of the receiver unit. For example, the receiver unit may be a desktop computer, a laptop computer, a tablet computer, a smartphone, or a TTY device. According to certain embodiments, the receiver is a tablet computer or smartphone, e.g., a tablet computer or smartphone which runs an operating system selected from an iOS^{TM} operating system, an Android™ operating system, a Windows^{TM} operating system, or any other tablet- or smartphone-compatible operating system.

Speech communication systems of the subject disclosure may include any components or functionalities described hereinabove with respect to the subject methods. For example, the may include the number, types, and positioning of one or more vocal tract articulators or processors as described above in regard to the methods of the present disclosure such that speech signals in response to the physiological feature sufficient to are detected and can be generated into audible speech. Also by way of example, the memory (e.g., a non-transitory computer readable medium) of the receiving unit may include instructions for performing time-frequency analysis (e.g., by Fast Fourier Transform (FFT), wavelet transform, Hilbert transform, bandpass filtering, and/or the like) of speech pattern signals, physiological feature signals, or acoustic signals can be detected and/or generated.

In some embodiments, the memory (e.g., a non-transitory memory) includes instructions for deriving a speech production signal pattern from the detected speech production signals and correlating the speech production signal pattern with a reference speech production signal pattern. In certain aspects, the speech production signal pattern is a spatiotemporal pattern of activity and/or inactivity in regions of the vSMC identified by the present inventor as corresponding to regions associated with the control of particular speech articulators. Upon establishment of reference speech production signal patterns (such as the spatiotemporal signal patterns) corresponding to various speech sounds (e.g., the various phonemes, syllables (e.g., CVs), words, parts of words, parts of sentences, and the like as described in detail in the Examples section below) which may be included in the same or a separate memory, the speech sound produced or imaginarily produced by the subject may be decoded by correlating the derived speech production signal pattern(s) to the reference speech production signal pattern(s). That is, the reference speech production signal pattern that correlates (e.g., is most similar with respect to the spatiotemporal activity pattern in the vSMC) to the derived speech production signal pattern may be identified. Upon identification of this reference speech production signal pattern, the derived speech production signal pattern is decoded as the speech sound associated with the reference speech production signal pattern, thereby decoding speech from the brain of the subject. In certain aspects, a decoding algorithm is trained on recorded data (e.g., a database of reference speech production signal patterns), stored in the memory, and then applied to novel neural signal inputs for real-time implementation.

### Systems—Speech Synthesis Decoding

Also provided are systems for performing the methods of the present disclosure. Such systems include speech decoding systems.

Aspects of the present disclosure include a system comprising an electrode array positioned on the brain of an individual; one or more procesors; a non-transient computer-readable medium comprising instructions that, when executed by the one or more processors, cause the one or more processors to: record neural signals associated with cortical activity in the brain; extract one or more neural signals of the brain; and decode a speech output from the neural signals.

Aspects of the present disclosure include a speech neural decoding system comprising an electrode array in contact with the cortical region of the brain in the individual, wherein the electrode array comprises a plurality of electrodes; an electrical recording device configured to record neural signals in the brain; one or more processors; a non-transient computer-readable medium comprising instructions that, when executed by the processor, cause the one or more processors to: perform one or more filters on the plurality of signals; decode the plurality of signals into articulatory movement representations; and output the plurality of acoustic signals into a speech output.

Aspects of the present disclosure include a speech neural decoding system comprising:

an electrode array in contact with the cortical region of the brain in the individual; one or more processors; a non-transient computer-readable medium comprising instructions that, when executed by the one or more processors, cause the one or more processors to: record neural or optical signals associated with cortical activity in the brain; extract one or more features associated with cortical activity in the brain; decode articulatory movement features from the one or more features of the neural signals; decode acoustic signals from the articulatory movement features, and decode a speech output from the acoustic signals.

In some embodiments, the neural array comprises a plurality of electrodes. In some embodiments, the plurality of electrodes comprises 50 or more electrodes, 100 or more electrodes, 150 or more electrodes, 200 or more electrodes, 250 or more electrodes, 300 or more electrodes, 350 or more electrodes, 400 or more electrodes, 450 or more electrodes, or 500 or more electrodes.

In some embodiments, the system comprises an electrical recording device configured to record neural signals in the brain. In some embodiments, the electrical recording device is a 16-channel recording device. In some embodiments, the electrical recording device is a 32-channel recording device. In some embodiments, the electrical recording device is a 64-channel recording device. In some embodiments, the electrical recording device is a 128-channel recording device. In some embodiments, the electrical recording device is an 256-channel recording device. In some embodiments, the electrical recording device is implantable. In some embodiments, the electrical recording device is wireless.

In some embodiments, the electrical recording device is an ECoG recording device. In some embodiments, the electrical recording device is an EEG recording device. In some embodiments, the electrical recording device comprises a plurality of microelectrodes. In some embodiments, the electrical recording device is any known electrical recording device configured to record a plurality of neural signals in the brain.

In some embodiments, the system comprises one or more processors. In some embodiments, the system comprises a non-transient computer-readable medium comprising instructions that, when executed by the processor, cause the one or more processors to: perform one or more filters on the plurality of signals; decode the plurality of signals into articulatory movement representations; and output the plurality of acoustic signals into a speech output.

In some embodiments, the plurality of signals comprise ECoG signals or EEG signals. In some embodiments, the ECoG signals or EEG signals are neural signals.

In some embodiments, the one or more filters comprises one or more low-pass filters (e.g. low frequency component ranging from 1-30 Hz). In some embodiments, the one or more filters comprises one or more notch filters.

In some embodiments, neural signals are filtered at a high gamma frequency ranging from 70 to 200 Hz. In some embodiments, the neural signals are filtered at a low frequency ranging from 1-30 Hz.

In some embodiments, the one or more processors is configured to stream the signals onto a computer, tablet, smartphone, and/or related devices.

wherein the one or more processors is configured to apply one or more band-pass finish impulse response (FIR) filters to the neural signals. In some embodiments, the one or more FIT filters are configured to band-pass the neural signals in one or more different sub-bands in the high gamma band frequency range. In some embodiments, the one or more processors is configured to extract analytic amplitude values (e.g. high gamma analytic amplitude values) across the one or more band-pass FIR filters applied to the neural signals. In some embodiments, the one or more processors is configured to average the analytic amplitude values across the one or more band-pass FIR filters to obtain one or more high gamma analytic amplitude signals.

In some embodiments, the one or more processors is configured to normalize and store the one or more high gamma analytic amplitude signals in an event detector process, wherein the event detector process analyzes the high gamma analytic signals at one or more time points. In some embodiments, the event detector process is configured to analyze the high gamma analytic signals at one or more time points to predict the onset and offset of auditory perceived speech or verbal produced speech events.

In some embodiments, the one or more processors is configured to decode the one or more high gamma analytic amplitude signals into an intended speech output.

In some embodiments, the electrode array is contacted with the cortical region of the brain. In some embodiments, the electrode array is positioned on a cap that is placed on the surface of the cortical region of the brain. In some embodiments, said contacting comprises implanting the electrode array in the cortical region of the brain. In some embodiments, wherein said contacting comprises operably coupling a neurosensor comprising the electrode array to the cortical region of the brain.

In some embodiments, the intended speech output is configured to output text as one or more syllables, words, parts of words, phrases, utterances, paragraphs, sentences, and/or a combination thereof.

Aspects of the present disclosure include a system comprising: an electrode array positioned on a brain of an individual; one or more processors; a non-transient computer-readable medium comprising instructions that, when executed by the one or more processors, cause the one or more processors to: record and/or detect neural signals associated with cortical activity in the brain; extract one or more features from the neural signals of the brain; decode articulatory movement features from the one or more features of the neural signals; decode acoustic signals from the articulatory movement features; and decode a speech output from the acoustic signals. In some embodiments, the speech output is text comprising one or more syllables, words, parts of words, phrases, utterances, paragraphs, sentences, and/or a combination thereof.

Aspects of the present disclosure include a system comprising an electrode array positioned on the brain of an individual; one or more procesors; a non-transient computer-readable medium comprising instructions that, when executed by the one or more processors, cause the one or more processors to: record neural signals associated with cortical activity in the brain; extract one or more neural signals of the brain; and decode a speech output from the neural signals.

Aspects of the present disclosure include a speech neural decoding system comprising an optical device configured to record optical signals from a cortical region of the brain in the individual; one or more processors; a non-transient computer-readable medium comprising instructions that, when executed by the processor, cause the one or more processors to: perform one or more filters on the plurality of signals; decode the plurality of optical signals into articulatory movement representations; and output the plurality of optical signals into a speech output.

Aspects of the present disclosure include a speech neural decoding system comprising:

an optical device configured to record optical signals from a cortical region of the brain in the individual; one or more processors; a non-transient computer-readable medium comprising instructions that, when executed by the one or more processors, cause the one or more processors to: record optical signals associated with cortical activity in the brain; extract one or more features associated with cortical activity in the brain; decode articulatory movement features from the one or more features of the optical signals; decode optical signals from the articulatory movement features, and decode a speech output from the optical signals.

In some embodiments, the system includes an optical device for configured to acquire optical signals associated with one or more context-related features. In some embodiments, the plurality of signals are acquired by any known neurophysiological recording device. In some embodiments, the plurality of signals are optical signals. In some embodiments, the plurality of signals are acquired through optical devices. Optical devices that can be used to acquire the plurality of signals include, but are not limited to: instrinic optical signal (I0S) imaging, extrinsic optical signal (EOS) imaging, Doppler flowmetry (LDF), near-infrared (NIR) spectrometer, functional optical coherence tomography (fOCT), and surface plasmon resonance (SPR). Other techniques such as radioactive imaging can be used to acquire the plurality of signals. Non-limiting examples include radioactive imaging of changes in blood flow, magnetoencephalography (MEG), thermal imaging, positron-emission tomography (PET), functional magnetic resonance imaging (fMRI), and diffuse optical tomography (DOT).

In some embodiments, the one or more processor comprises one or more BLSTM neural networks. In some embodiments, the one or more bidirectional long short-term memory comprises an algorithm for decoding articulatory movement features from the neural or optical signals. In some embodiments, the one or more bidirectional long short-term memory neural networks comprises an algorithm for decoding the acoustic signals, neural signals, and/or optical signals into text. In some embodiments, the one or more bidirectional long short-term memory neural networks comprising an algorithm for decoding articulatory movement features from the neural signals or optical signals is a first bidirectional long short-term memory neural network. In some embodiments, the one or more bidirectional long short-term memory neural networks comprising an algorithm for decoding the acoustic signals into text is a second neural network. In some embodiments, the second neural network is a bidirectional long short-term memory neural network. In some embodiments, the neural signals are electrocorticography (ECoG) neural signals. In some embodiments, the neural signals are EEG signals.

In some embodiments, the one or more processors comprises a second neural network (e.g. bidirectional long short-term memory) comprising an algorithm for decoding acoustic signals from the articulatory movement features. In some embodiments, the articulatory movement features comprise kinematic representations of articulation from the one or more features from the neural or optical signals. In some embodiments, the one or more processors comprises a second neural network (e.g. bidirectional long short-term memory)comprising an algorithm for decoding the audible speech and/or silent speech acoustic signals from the individual.

In some embodiments, the neural signals are recorded during an audible speech event, a silent speech event, and/or one or more external context-related cues from the individual. In some embodiments, the one or more processors is further configured to record the audible speech or silent speech signals from the individual.

In some embodiments, the one or more processors is further configured to record audible and silent speech signals simultaneously during recording of the neural signals.

In some embodiments, the one or more features of the neural signals comprises high-gamma amplitude signals in a frequency ranging from 70-200 Hz. In some embodiments, the one or more features of the neural signals comprises low frequency amplitude signals in a frequency ranging from 1-30 Hz.

In some embodiments, the one or more processors is configured to estimate vocal kinematic trajectories associated with the audible speech or silent speech signals.

In some embodiments, the one or more processors further comprises an autoencoder configured to convert phonological and acoustic features of the audible speech or silent speech acoustic signals into one or more articulatory representations. In some embodiments, the one or more processors further comprises a decoder configured to convert the one or more articulatory representations to audible speech or silent speech acoustic signals. In some embodiments, the one or more processors further comprises an encoder configured to estimate final articulatory kinematic features, wherein the final articulatory kinematic features are used in an algorithm to decode articulatory movement features from the neural signals.

In some embodiments, the electrode array is operably connected to the ventral sensorimotor cortex (vSMC), superior temporal gyrus (STG), and/or the inferior frontal gyms (IFG) of the brain.

In some embodiments, the one or more processors comprises a deep neural network comprising an algorithm for decoding the audible speech or silent speech acoustic signal as mel frequency cepstral coefficients. In some embodiments, the deep neural network comprises an algorithm for decoding the audible speech or silent speech acoustic signals as 25 dimensional mel frequency cepstral coefficients.

In some embodiments, the one or more processors comprises a hidden Markov model based acoustic model configured to perform sub-phonetic alignment.

In some embodiments, the one or more processors comprises a Kullback-Leibler

(KL) divergence model configured to compare the distribution of a decoded phoneme of the neural signals to a distribution of a ground-truth phoneme.

Aspects of the present disclosure include a speech neural decoding system comprising:

an electrode array in contact with the cortical region of the brain in the individual, wherein the electrode array comprises a plurality of electrodes; an electrical recording device configured to record neural signals in the brain; one or more processors; a non-transient computer-readable medium comprising instructions that, when executed by the processor, cause the one or more processors to: perform one or more filters on the plurality of signals; decode the plurality of signals into articulatory movement representations; and output the plurality of acoustic signals into a speech output.

In some embodiments, the one or more filters comprises one or more low-pass filters.

In some embodiments, wherein the one or more filters comprises one or more notch filters.

In some embodiments, wherein the one or more processors is configured to stream the signals onto a real-time computer.

In some embodiments, wherein the neural signals are neural signals.

In some embodiments, wherein the one or more processors is configured to apply one or more band-pass finish impulse response (FIR) filters to the neural signals.

In some embodiments, wherein the one or more FIT filters are configured to band-pass the ECoG signals in one or more different sub-bands in the high gamma band frequency range.

In some embodiments, wherein the one or more processors is configured to extract analytic amplitude values across the one or more band-pass FIR filters applied to the neural signals.

In some embodiments, wherein the one or more processors is configured to average the analytic amplitude values across the one or more band-pass FIR filters to obtain one or more high gamma analytic amplitude signals.

In some embodiments, wherein the one or more processors is configured to normalize and store the one or more high gamma analytic amplitude signals in an event detector process, wherein the event detector process analyzes the high gamma analytic signals at one or more time points.

In some embodiments, wherein the event detector process analyzes the high gamma analytic signals at one or more time points to predict the onset and offset of auditory perceived speech or verbal produced speech events.

In some embodiments, wherein the one or more processors is configured to decode the one or more high gamma analytic amplitude signals into an intended speech output.

In some embodiments, wherein said contacting comprises implanting the electrode array in the cortical region of the brain.

In some embodiments, wherein said contacting comprises operably coupling a neurosensor comprising the electrode array to the cortical region of the brain.

In some embodiments, wherein the intended speech output is configured to output text as one or more syllables, words, parts of words, phrases, utterances, paragraphs, sentences, and/or a combination thereof.

In some embodiments, wherein the neural signals are filtered at a frequency ranging from 70 to 200 Hz.

In some embodiments, wherein the one or more processor comprises one or more bidirectional long short-term memory (BLSTM) or other recurrent neural networks.

A system comprising: an electrode array positioned on a brain of an individual;

one or more processors; a non-transient computer-readable medium comprising instructions that, when executed by the one or more processors, cause the one or more processors to: record neural signals associated with cortical activity in the brain; extract one or more features from the neural signals of the brain; decode articulatory movement features from the one or more features of the neural signals; decode acoustic signals from the articulatory movement features; and decode a speech output from the acoustic signals.

In some embodiments, wherein the one or more processors comprises a recurrent neural network (RNN).

In some embodiments, wherein the RNN is one or more bidirectional long short- term memory (BLSTM) or other recurrent neural networks.

In some embodiments, wherein the bidirectional long short-term memory comprises an algorithm for decoding articulatory movement features from the neural signals.

In some embodiments, wherein the one or more bidirectional long short-term memory neural networks comprises an algorithm for decoding the acoustic signals into text.

In some embodiments, wherein the speech output is text comprising one or more syllables, words, parts of words, phrases, utterances, paragraphs, sentences, and/or a combination thereof.

In some embodiments, wherein the one or more processors comprises a second neural network (e.g. bidirectional long short-term memory) comprising an algorithm for decoding acoustic signals from the articulatory movement features

In some embodiments, wherein the articulatory movement features comprise kinematic representations of articulation from the one or more features from the neural signals.

In some embodiments, wherein the neural signals are recorded during:

audible speech from the individual;

silent or intended speech from the individual; and/or

a sound heard from the individual.

In some embodiments, wherein the one or more processors is further configured to record audible or silent speech signals simultaneously during recording of the neural signals.

In some embodiments, wherein the neural signals are electrocorticography (ECoG) neural signals.

In some embodiments, wherein the one or more features of the neural signals comprises high-gamma amplitude signals in a frequency ranging from 70-200 Hz.

In some embodiments, wherein the one or more features of the neural signals comprises low frequency amplitude signals in a frequency ranging from 1-30 Hz.

In some embodiments, wherein the one or more processors is further configured to record the audible speech or silent speech signals from the individual.

In some embodiments, wherein the one or more processors comprises a second neural network (e.g. bidirectional long short-term memory) comprising an algorithm for decoding the audible speech or silent speech acoustic signals from the individual.

In some embodiments, wherein the one or more processors is configured to estimate vocal kinematic trajectories associated with the audible speech or silent speech signals.

In some embodiments, wherein the one or more processors further comprises an autoencoder configured to convert phonological and acoustic features of the audible speech or silent speech acoustic signals into one or more articulatory representations.

In some embodiments, wherein the one or more processors further comprises a decoder configured to convert the one or more articulatory representations to audible speech or silent speech acoustic signals.

In some embodiments, wherein the one or more processors further comprises an encoder configured to estimate final articulatory kinematic features, wherein the final articulatory kinematic features are used in an algorithm to decode articulatory movement features from the neural signals.

In some embodiments, wherein the electrode array is operably connected to the ventral sensorimotor cortex (vSMC), superior temporal gyms (STG), and the inferior frontal gyrus (IFG) of the brain.

In some embodiments, wherein the one or more processors comprises a deep neural network comprising an algorithm for decoding the audible speech or silent speech acoustic signal as mel frequency cepstral coefficients.

In some embodiments, wherein the deep neural network comprises an algorithm for decoding the audible speech or silent speech acoustic signals as 25 dimensional mel frequency cepstral coefficients.

In some embodiments, wherein the one or more processors comprises a hidden Markov model based acoustic model configured to perform sub-phonetic alignment.

In some embodiments, wherein the one or more processors comprises a Kullback-Leibler (KL) divergence model configured to compare the distribution of a decoded phoneme of the ECoG signals to a distribution of a ground-truth phoneme.

A system comprising: an electrode array positioned on a brain of an individual; one or more processors; a non-transient computer-readable medium comprising instructions that, when executed by the one or more processors, cause the one or more processors to: record neural signals associated with cortical activity in the brain; extract one or more features from the neural signals of the brain; and decode a speech output from the neural signals.

In some embodiments, wherein the processor further decodes articulatory movement features from the one or more features of the neural signals.

In some embodiments, wherein the processor further decodes acoustic signals from the articulatory movement features.

### Utility

The subject methods and systems find use in any application in which it is desirable to decode speech from the brain of a subject (e.g., a human subject). Subjects of interest include those in which the ability to communicate via spoken language is lacking or impaired. Examples of such subjects include, but are not limited to, subjects who may be suffering from paralysis, locked-in syndrome, Lou Gehrig's disease, aphasia, dysarthria, stuttering, laryngeal dysfunction/loss, vocal tract dysfunction, and the like. An example application in which the subject methods and systems find use is providing a speech impaired individual with a speech communication neuroprosthetic system which detects and decodes speech production signals and/or patterns thereof from the speech motor cortex of the subject and produces audible speech and/or speech in text format, enabling the subject to communicate with others without using speech articulators or writing/typing the speech for display to others. The methods and systems of the present disclosure also find use in diagnosing speech motor disorders (e.g., aphasia, dysarthria, stuttering, and the like). In addition, the subject methods and systems find use, e.g., in enabling individuals to communicate via mental telepathy.

In certain aspects, methods and systems of the present disclosure utilize population neural analyses to decode and/or generate individual speech sounds (phonemes, including consonants and vowels). These speech sounds are the building block units of human speech. Phonemes can be concatenated into syllables, words, phrases and sentences to provide the full combinatorial potential of spoken language. This approach based on the natural neurophysiologic mechanisms of speech production has distinct advantages over present technologies for, e.g., communication neuroprostheses, which either focus on purely acoustic parameter control (e.g. formant) or spelling devices, neither of which are robust or efficient for communication.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Efforts have been made to ensure accuracy with respect to numbers used (e.g. amounts, temperature, etc.) but some experimental errors and deviations should be accounted for.

### Example 1

### Generative Modeling of Human Speech Production Using Articulatory Physiology

The following presents a framework for analysis and synthesis of speech by mimicking the generative process of articulatory physiological behavior in human speech production. The present disclosure reliably estimates the articulatory physiological substrate from the speech acoustic signal (i.e., the ‘speech motor code’). Computationally, a deep recurrent encoder decoder architecture is implemented to encode phonological and acoustic signals into an ‘articulatory physiological embedding’ that decodes the speech acoustics. The stacked network jointly optimizes the physiological representation and the generated acoustic signal. The embedding was validated as the true physiological substrate empirically by showing performance in acoustic-to-articulatory inversion. Additionally, a new generative text-to-speech system was created that performs the 2-stage conversion of text into a physiological embedding that is then converted to acoustics. It was shown that enforcing the physiological intermediate yields better quality synthesis while requiring lesser amount of training data than is conventionally demanded by current models for speech synthesis.

The present disclosure provides for speech production datasets based on Electromagnetic Midsagittal articulography (EMA) and a method for inferring the physiological substrate for the speech signal and show objective gains of such a representation in speech synthesis.

### Speech Production: Integrating Phonology, Physiology and Acoustics

Though the speech communication process is cognitively symbolic (i.e., lexical and phonological) within the speaker and the listener, the underlying phonological string uttered by a speaker is realized and executed as a continuous motor sequence where the ventral sensorimotor cortex mediates the coarticulated, multi-articulator spatiotemporal movements of the vocal tract articulators. These physiological movements add higher order resonances to the acoustic source of air expelled through vibrating vocal cords. The resulting acoustic signal is then perceived by the listener in the auditory cortex in terms of the phonetic features of the incoming acoustic stream

Speech is conventionally represented at these two ‘observable’ levels of abstraction—(i) the phonological level which describes the signal in terms of phonemes, syllables and their properties, and as the (ii) the acoustic signal, a continuous time domain or spectrotemporal representation of the acoustic resonances as produced by the speaker. Articulatory physiology is the ‘latent’ process that links these two levels in speech production (phonology→physiology→acoustics). Since most of the articulatory processes are within the oropharyngeal and nasal cavity, and happen at rapid time scales, they remain hidden to the naked eye or any imaging modality. Existing efforts to assay the articulatory processes underlying speech production through techniques like X-ray microbeam, Electromagnetic Midsagittal Articulography, and more recently real time Magnetic Resonance Imaging, offer a spatially and temporally sparse and incomplete view of the physiological processes making it hard to model the explicit generative process. Hence conventional ‘acoustic models’ are predictive distributions of acoustic observations over phonological units. Following formulation prescribed in, given a training data set of acoustics X and corresponding word sequences W, the acoustic observation sequence for a given word w is drawn from the posterior predictive distribution by introducing the auxiliary variable λ,

P(x|w, X, W)=∫P(x, λ|w, X, W)dλ=∫P(x|w, λ)P(λ|W, X)dλ (1)

The two factors are individually optimized for computational tractability, where in the training phase, acoustic model \hat{λ} is chosen as,

$$\begin{matrix}\hat{λ} = {argmax}_{λ} P ( λ \vert X , W ) & ( 2 ) \\ = {argmax}_{λ} P ( λ \vert X , L ) P ( L \vert W ) & ( 3 )\end{matrix}$$

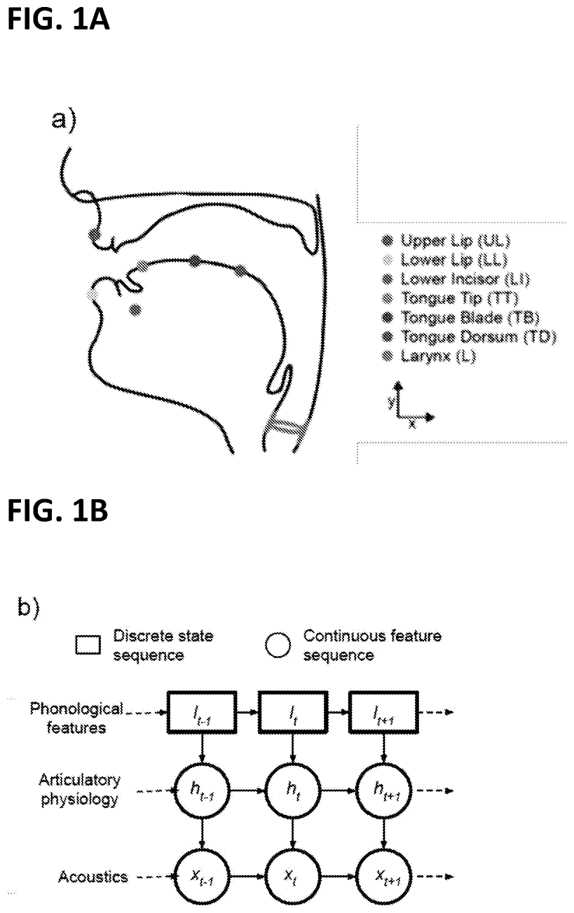

sequences W are further expanded as a Markov sequences of the phonological states L, that factors out the dependency on words. Similarly the predictions are made at test time by drawing from P(x|\hat{l}, \hat{λ}) where \hat{l}=arg maxₗ P(l|w), is the predicted Markov sequence of the phonetic states. Even as efforts in statistical parametric speech synthesis continue to improve, they do not approach the naturalness of more concatenative nonparametric strategies. This statistical paradigm was improved by explicitly including the physiology H as the dependency that factors the phonology L out of the acoustic model. The graphical model illustration is given in FIG. 1. Specifically, a refactorization of the acoustic model is provided herein as

$$\hat{λ} = {arg max}_{λ} P ( λ \vert X , H ) P ( H \vert L ) P ( L \vert W ) \qquad ( 4 )$$

and the prediction as a draw from the distribution P(x|ĥ, \hat{λ}), where is the optimal physiological description for a given phonological string l

$$\hat{h} = {arg max }_{h} P ( h \vert l ) . \qquad ( 5 )$$

The parameters that demonstrated physiology H made the acoustic model λ independent of phonology L, included choosing H that i) was predictable from phonology, to maximize P(H|L), and ii) provide a complete account of the acoustic signal, to maximize P(w|ĥ, \hat{λ}).

Optimizing H, The Physiological Substrate of the Speech Signal

An encoder-decoder architecture for a deep network to jointly optimize these constraints is shown. An aspect of the present disclosure was to learn a physiological embedding for speech data. Since articulatory physiology is a complex motor behavior with a many to one mapping between articulation and acoustics, it is important to use actual physiological data. The Electromagnetic Midsagittal Articulography (EMA) dataset was used that has parallel recordings of acoustics and displacement traces in caudo-rostral x and y directions of select points on a subject's vocal tract as shown in FIG. 1A. For the EMA data, the displacement traces capture the shaping of the vocal tract and places of articulation. One property of speech is the manner of consonant constriction, i.e., whether the consonant is a plosive, lateral, fricative, or nasal etc. An aspect of the present disclosure was to reconstruct the speech signal with minimal loss, additional manner information was augmented to create a representation for the speech signal that is has the representational capacity for complete description of speech. Since manner is manifest in acoustics, manner feature detectors were trained on existing acoustic speech corpora. These detectors are then run on the EMA subject to create the manner of articulation feature streams, synchronously with the EMA and the acoustic data. Specifically, binary feature detectors of place and manner are trained on wall street journal speech corpus. Additional physiological information about energy and voicing etc., are also included. Note that while the fundamental frequency F0 is certainly physiological, it is solely caused by the vocal folds and not considered in this study. In all 31 continuous valued features are created for all utterances of the corpus. These features serve as the initialization for the physiological substrate of the speech signal. FIG. 3 presents an example utterance along with the spectrogram and the initialized physiological features. The EMA displacements are real data collected from the articulatory kinematic movements during this utterance. The manner feature streams are very spare and are invoked only when the associated manner is evident from the acoustics. Encoder-decoder network architectures can be used for learning meaningful embeddings in several domains. In order to enable a physiological encoding, encode phonological features were also encoded along with the acoustic features into the 31 dimensional feature space. Since physiology is coarticulated both with carryover and anticipatory coarticulation, it is important for the encoder network to be a recurrent network. State-of-the-art sequence-to-sequence regression was used with bidirectional LSTM cells to encode the physiological layer. Similarly, a decoder was trained to go from the physiological descriptions to acoustic observations, coded as 25 dimensional mel frequency cepstral coefficients. Since articulation can causally account for acoustics in the paradigm provided herein, a strictly feedforward network was used as the decoder. Once these networks are trained individually, they are stacked together and backpropagated through the whole training data as a single network as illustrated in FIG. 3.

Since initialization alone doesn't ensure that the eventual bottleneck layer ends up being “physiological”, two loss functions were optimized jointly—i) Mean squared error on the reconstructed acoustic signal, and an auxiliary loss function i.e., ii) Mean squared error on the EMA displacement traces in the bottleneck layer for which there is groundtruth data. The augmented manner features are not included in the cost function allowing the network to freely change them through the backpropagation training

Is the Embedding Truly Physiological?

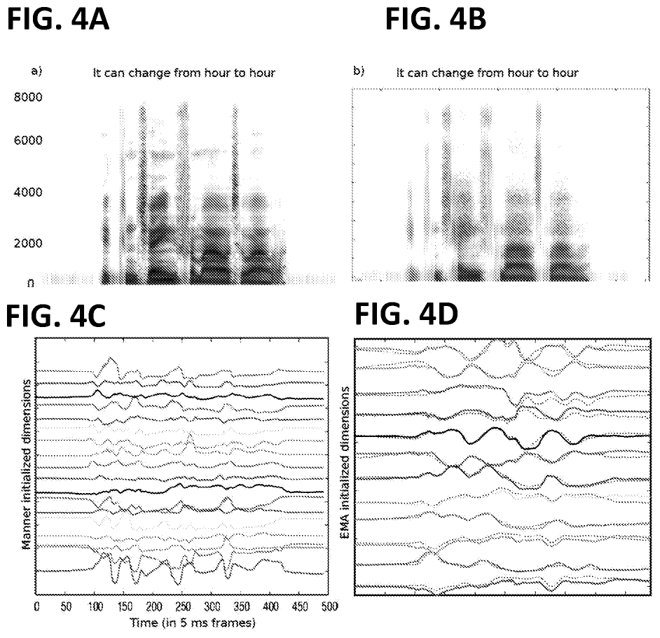

The algorithm provided herein is run on the training set of the mngu0 corpus. Upon completion of training, unseen utterances were encoded using the stacked network. FIGS. 4A-4D shows an example test utterance and the outputs of the encoding layer and decoding layers.

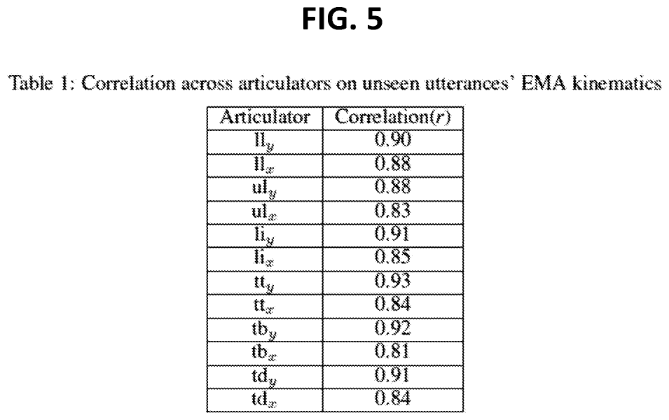

The decoded spectrogram reconstructions of unseen trials are completely intelligible and have minimal perceptual loss to the original utterances. Furthermore, the encoded embedding was found to match with very high degree of correlation to the EMA trajectories of the subjects' actual productions as can be seen in FIG. 4D, confirming that the encoder network is embedding a physiological substrate of the actual speech signal. The augmented manner dimensions change post training, in that they are smoother and relatively less sparse than initialized, but still adhere to the broad class they were initialized to capture. The summary statistics of all articulator correlations on test set is shown in table 1 for the EMA dimensions where ground truth is available is shown in Table 1 of FIG. 5.

A Physiologically Generative Model of Speech Synthesis

The ability to infer a reliable physiological embedding H lets allowed for generative modelling of acoustics X, and testing if such an endeavor has any benefits compared to the traditional acoustics based on phonology L. To investigate this, all training data from the mngu0 corpus was processed to estimate the physiological embedding using approaches described earlier. Two models of speech synthesis are compared, i) deep bidirectional LSTM based network for estimating acoustics from phonological features (Traditional TTS) and ii) A similar network to perform the 2-stage process of physiological substrate from phonological features, followed by conversion of physiology to acoustics.

To further validate the hypothesis that generative modelling of the causal physiological process reduced the need for data compared to a completely agnostic but similar network architecture, performance was reported where only 16 minutes of speech is made available to the synthesizers. The Mel-Cepstral Distortion, an error metric used to check goodness of an acoustic model is compared. It can be seen that, indeed imposition of the physiology factorization is able to give better prediction than a conventional acoustic model for speech synthesis based only on phonological features. The difference in performance is more striking when very little amount of training data is presented.

Provided herein is a generative modelling of the causal physiological processes in the context of speech production. After reformulating the conventional statistical paradigm for speech synthesis to explicitly model physiology, an algorithm was developed for inferring the physiological substrate of the speech signal that was validated on ground truth behavior. Additionally, the benefit of such a reformulation was shown by gains in statistical speech synthesis. Physiological modelling can be beneficial to several other speech problems like voice conversion, articulatory synthesis, speech recognition as it is modelling the very coarticulatory processes that make acoustic modelling challenging in these applications.

### Example 2

### Encoding of Articulatory Kinematic Trajectories in Human Speech Sensorimotor Cortex

Encoding of articulatory kinematic trajectories in human speech sensorimotor cortex is shown in Chartier et al., (Chartier et al., (2018) Neuron 98, 1042-1054), which is hereby incorporated by reference in its entirety.

Fluent speech production requires precise vocal tract movements. The encoding of these movements in the human sensorimotor cortex was examined Neural activity at individual electrodes encodes diverse movement trajectories that yield the complex kinematics underlying natural speech production.

High-density intracranial electrocorticography (ECoG) signals were recorded while participants spoke aloud in full sentences. Continuous speech production provided for studying the dynamics and coordination of articulatory movements not well captured during isolated syllable production. Furthermore, since a wide range of articulatory movements is possible in natural speech, sentences were used to cover nearly all phonetic and articulatory contexts in American English. This approach provided herein allowed characterization of sensorimotor cortical activity during speech production in terms of vocal tract movements.

A statistical approach was developed to derive the vocal tract movements from the produced acoustics. The inferred articulatory kinematics were used to determine the neural encoding of articulatory movements, in a manner that was model independent and agnostic to pre-defined articulatory and acoustic patterns used in speech production (e.g., phonemes and gestures). By learning how combinations of articulator movements mapped to electrode activity, articulatory kinematic trajectories (AKTs) were estimated for single electrodes and characterized the heterogeneity of movements that were represented through the speech vSMC.

Inferring Articulatory Kinematics

To estimate the articulatory kinematics during natural speech production, reliable estimates of vocal tract movements were obtained from only the produced speech acoustics.

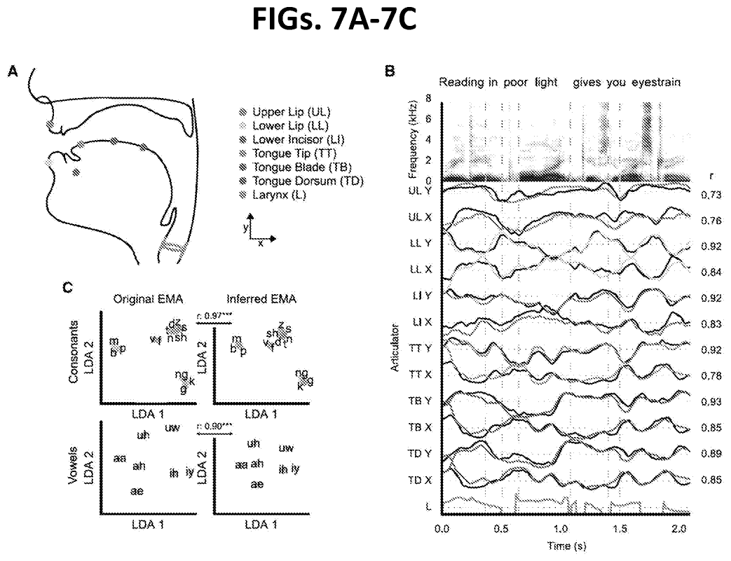

Provided herein is an approach for speaker-independent AAI. The AAI model was trained using publicly available multi-speaker articulatory data recorded via EMA, a reliable vocal tract imaging technique well suited to study articulation during continuous speech production. The training dataset comprised simultaneous recordings of speech acoustics and EMA data from eight participants reading aloud sentences from the MOCHA-TIMIT dataset . EMA data for a speech utterance consisted of six sensors that tracked the displacement of articulators critical to speech articulation (FIG. 7A) in the caudorostral (x) and dorsoventral (y) directions. Laryngeal function was approximated by using the fundamental frequency (f0) of produced acoustics and whether or not the vocal folds were vibrating (voicing) during the production of any given segment of speech. In all, a 13 dimensional feature vector described articulatory kinematics at each time point (FIG. 7B).

Phonological context was incorporated into a deep neural network to capture context-dependence variance.

Additionally, training speakers were spectrally warped to sound like the target (or test) speaker to improve cross-speaker generalizability. With these modifications, the AAI method provided herein performed markedly better than the current state-of-the-art methods within the speaker-independent condition and proved to be a reliable method to estimate articulatory kinematics. Using leave-one-participant-out cross-validation, the mean correlation of inferred trajectories with ground truth EMA for a held out test participant was 0.68±0.11 across all articulators and participants (0.53 correlation reported by Afshan and Ghosh, 2015). FIG. 7B shows the inferred and ground truth EMA traces for each articulator during an example utterance for an unseen test speaker. There was a high degree of correlation across all articulators between the reference and inferred movements. Figure S1A shows a detailed breakdown of performance across each of the 12 articulators. To investigate the ability of AAI method presented herein to infer acoustically relevant articulatory movements, identical deep recurrent networks were trained to perform articulatory synthesis, i.e., predicting the acoustic spectrum (coded as 24-dimensional mel-cepstral coefficients and energy) from articulatory kinematics, for both the real and inferred EMA. It was found on average that there was no significant difference (p=0.4; Figures S1B and S1C) in the resulting acoustic spectrum of unseen utterances when using either the target speaker's real EMA or those inferred via from the AAI method. This suggests that the difference between inferred and real EMA may largely be attributed to kinematic excursions that do not have significant acoustic effects. Other factors may also include differences in sensor placement, acquisition noise, and other speaker/recording specific artifacts that may not have acoustic relevance.

To further validate the AAI method, how well the inferred kinematics preserved phonetic structure was evaluated. To do so, the phonetic clustering resulting from both real and inferred kinematic descriptions of phonemes was analyzed. For one participant's real and inferred EMA, a 200-ms window of analysis was constructed around the kinematics for each phoneme onset. Linear discriminant analysis (LDA) was used to model the kinematic differences between phonemes from the real EMA data. Both real and inferred EMA data was projected for phonemes into this two-dimensional LDA space to observe the relative differences in phonetic structure between real and inferred EMA. It was found that the phonetic clustering and relative distances between phonemes centroids were largely preserved (FIG. 7C) between inferred and real kinematic data (correlation r=0.97 for consonants and 0.9 for vowels; p<0.001). Together, these results demonstrate that using kinematic, acoustic, and linguistic metrics, it is possible to obtain high-resolution descriptions of vocal tract movements from easy-to-record acoustic data.

Encoding of Articulatory Kinematic Trajectories at Single vSMC Electrodes

Using AAI, vocal tract movements were inferred as traces from EMA sensor locations (FIG. 7A) while participants read aloud full sentences during simultaneous recording of acoustic and high-density intracranial ECoG signals. To describe the relationship between vocal tract dynamics and sensorimotor cortical activity, a trajectory-encoding model was used to predict each electrode's high gamma (70-150 Hz) activity (Z scored analytic amplitude) as a weighted sum of articulator kinematics over time. Ridge regression was used to model high gamma activity for a given electrode from time-varying estimated EMA sensor positions.

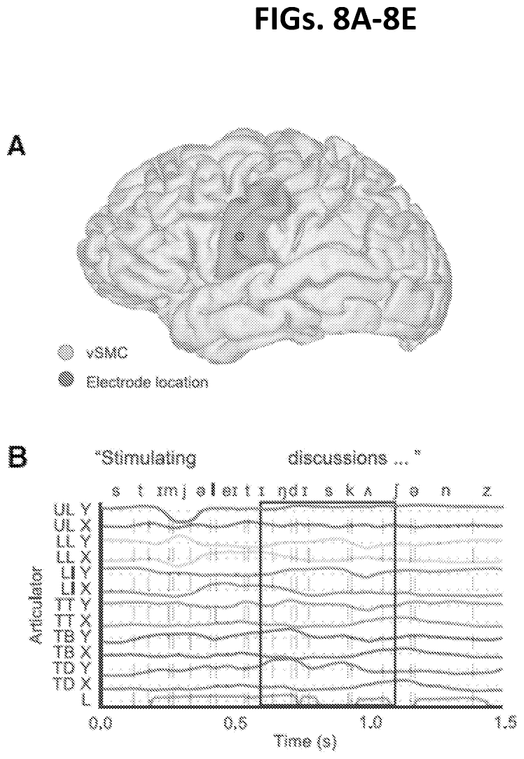

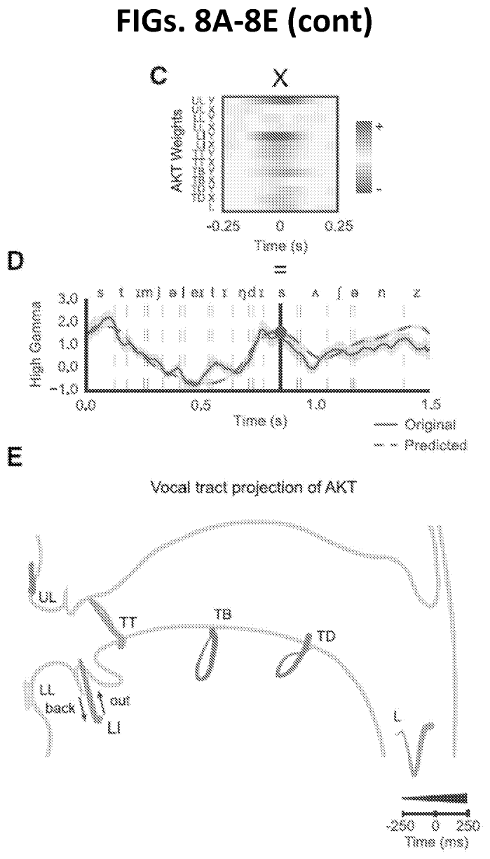

In FIG. 8, for an example electrode (FIG. 8A) it was shown that the weights learned (FIG. 8C) from the linear model act as a spatiotemporal filter that was then convolved with articulator kinematics (FIG. 8B) to predict electrode activity (FIG. 8D). The resulting filters described specific patterns of AKTs (FIG. 8C), which are the vocal tract dynamics that best explain each electrode's activity. By validating on held-out data, it was found that the AKT model significantly explained neural activity for electrodes active during speech in the vSMC (108 electrodes across 5 participants; mean r=0.25±0.08 up to 0.5, p<0.001) compared to AKT models constructed for electrodes in other anatomical regions (p<0.001, Wilcoxon signed-rank test; Figure S7).

To provide a more intuitive understanding of these filters, the X and Y coordinates were projected of each trajectory onto a midsagittal schematic view of the vocal tract (FIG. 8E). Each trace represents a kinematic trajectory of an articulator with a line that thickens with time to illustrate the time course of the filter.

For the special case of the larynx, voicing-related pitch modulations were used that were represented along the y axis with the x axis, providing a time course for visualization.

A consistent pattern was observed across articulators in which each exhibited a trajectory that moved away from the starting point in a directed fashion before returning to the starting point. The points of maximal movement describe a specific functional vocal tract shape involving the coordination of multiple articulators. For example, the AKT (FIG. 8E) for the electrode in FIG. 8A exhibits a clear coordinated movement of the lower incisor and the tongue tip in making a constriction at the alveolar ridge. Additionally, the tongue blade and dorsum move frontward to facilitate the movement of the tongue tip. The upper and lower lips remain open and the larynx is unvoiced. The vocal tract configuration corresponds to the classical description of an alveolar constriction (e.g., production of /t/, /d/, /s/, /z/, etc.). The tuning of this electrode to this particular phonetic category is apparent in FIG. 8D, where both the measured and predicted high gamma activity increased during the productions /st/, /dis/, and /nz/, all of which require an alveolar constriction of the vocal tract.

While vocal tract constrictions have typically been described as the action of one primary articulator, the coordination among multiple articulators is critical for achieving the intended vocal tract shape. For example, in producing a /p/, if the lower lip moves less than it usually does (randomly, or because of an obstruction), then the upper lip compensates and lip closure is accomplished. This coordination may arise from the complex and highly overlapping topographical organization of articulator representation in the vSMC.

Alternatively, high gamma activity could be related to a single articulator trajectory with the rest of articulators representing irrelevant correlated movements. To evaluate these hypotheses, a cross-validated, nested regression model was used to compare the neural encoding of a single articulator trajectory with the AKT model. Here, one articulator was referred to as one EMA sensor. The models were trained on 80% of the data and tested on the remaining 20% data. For each electrode, fit single articulatory trajectory models were fit using both x and y directions for each estimated EMA sensor and chose the single articulator model that performed best for the comparison with the AKT model. Since each single articulator model is nested in the full AKT model, a general linear F-test was used to determine whether the additional variance explained by adding the rest of the articulators at the cost of increasing the number of parameters was significant. After testing each electrode on the data held-out from the training set, it was found that the multi-articulatory patterns described by the AKT model explained significantly more variance compared to the single articulator trajectory model (F(280,1,820)>1.31, p<0.001 for 96 of 108 electrodes, mean F-statistic=6.68, p<0.001, Wilcoxon signed-rank tests; Figure S3; mean change in R2, 99.55%±8.63%; Figure S4). This means that activity of single electrodes is more related to vocal tract movement patterns involving multiple articulators than it is to those of a single articulator.

One potential explanation for this result is that single electrode neural activity in fact encodes the trajectory of a single articulator but could appear to be multi-articulatory because of the correlated movements of other articulators due to the biomechanical properties of the vocal tract. The structure of correlations were examined among articulators during periods of high and low neural activity for each speech-active electrode. If the articulator correlation structures were the same regardless of electrode activity, then the additional articulator movements would be solely the result of governing biomechanical properties of the vocal tract. However, it was found that articulator correlation structures differed according to whether high gamma activity was high or low (threshold at 1.5 SDs) (p<0.001 for 108 electrodes, Bonferroni corrected), indicating that in addition to coordination due to biomechanical properties of the vocal tract, coordination among articulators was reflected in changes of neural activity. Contrary to popular assumptions of a one-to-one relationship between a given cortical site and articulator in the homunculus, these results demonstrate that, similar to cortical encoding of coordinated movements in limb control, neural activity at a single electrode encodes the specific, coordinated trajectory of multiple articulators.

Kinematic Organization of vSMC

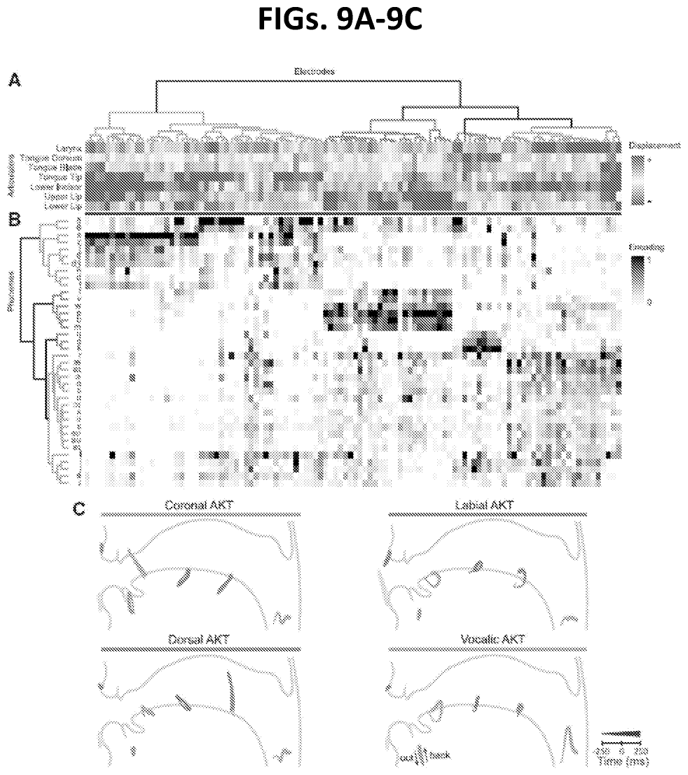

Hierarchical clustering of electrode selectivity patterns was used to reveal the phonetic organization of the vSMC. Whether clustering based upon all encoded movement trajectories (i.e., grouping of kinematically similar AKTs) yielded similar organization was then examined Because the AKTs were mostly out-and back in nature, the point of maximal displacement was extracted for each articulator along their principal axis of movement to concisely summarize the kinematics of each AKT. Hierarchical clustering was used to organize electrodes by their condensed kinematic descriptions (FIG. 9A).

To interpret the clusters in terms of phonetics, a phoneme-encoding model was fit for each electrode. Similar to the AKT model, electrode activity was explained as a weighted sum of phonemes in which the value each phoneme was either 1 or 0 depending on whether it was being uttered at a given time. For each electrode, the maximum encoding weight was extracted for each phoneme. The encoded phonemes for each electrode were shown in the same order as the kinematically clustered electrodes (FIG. 9B).

There was a clear organizational structure that revealed shared articulatory patterns among AKTs. The first level organized AKTs by their direction of jaw movement (lower incisor goes up or down). Sublevels manifested four main clusters of AKTs with distinct coordinative articulatory patterns. The AKTs in each cluster were averaged together, yielding a representative AKT for each cluster (FIG. 9C). Three of the clusters described constrictions of the vocal tract: coronal, labial, and dorsal, which broadly cover all consonants in English. The other cluster described a vocalic (vowel) AKT involving laryngeal activation and a jaw opening motion. Instead of distributed patterns of electrode activity representing individual phonemes, it was found that electrodes exhibited a high degree of specificity toward a particular group of phonemes.

Electrodes within each AKT cluster also primarily encoded phonemes that had the same canonically defined place of articulation. For example, an electrode within the coronal AKT cluster was selective for /t/, /d/, /n/, /∫/, /s/, and /z/, all of which have a similar place of articulation. However, there were differences within clusters. For instance, within the coronal AKT cluster (FIGS. 3A and 3B, green), electrodes that exhibited a comparatively weaker tongue tip movement (less purple) had phonetic outcomes less constrained to phonemes with alveolar places of constriction (less black for phonemes in green cluster).

Hierarchical clustering was also performed on the phoneme encoding weights to identify phoneme organization to both compare with and help interpret the clustering of AKTs. These results show phonetic organization of the vSMC, as phonetic features defined by place of articulation were dominant A strong similarity in clustering was found when electrodes were described by their AKTs and phonemes (FIGS. 3A and 3B), which is not surprising given that AKTs reflected specific locations of vocal tract constrictions (FIG. 9C).

Broad groupings of electrodes that were sensitive to place of articulation was observed, but within those groupings, differences in encoding for manner and voicing in consonant production were found. Within the coronal cluster, electrode-encoding weights were highest for fricatives, then affricates, and followed by stops (F(3)=36.01, p<0.001, ANOVA). Conversely, bilabial stops were more strongly encoded than labiodental fricatives (p<0.001, Wilcoxon signed-rank tests). Additionally, consonants (excluding liquids) were found to be clustered entirely separately from vowels. Again, the vocalic AKTs were defined by both laryngeal action (voicing) and jaw opening configuration. Vowels were organized by three primary clusters that correspond to low vowels, mid/high vowels, and high front vowels.

To understand how kinematically and phonetically distinct each AKT cluster was from one another, the relationship between within-cluster and between-cluster similarities was quantified for each AKT cluster using the silhouette index as a measure of clustering strength (Figure S5). The degrees of clustering strength of AKT clusters for kinematic and phonetic descriptions were significantly higher compared to shuffled distributions indicating that clusters had both similar kinematic and phonetic outcomes (p<0.01, Wilcoxon signed- rank tests).

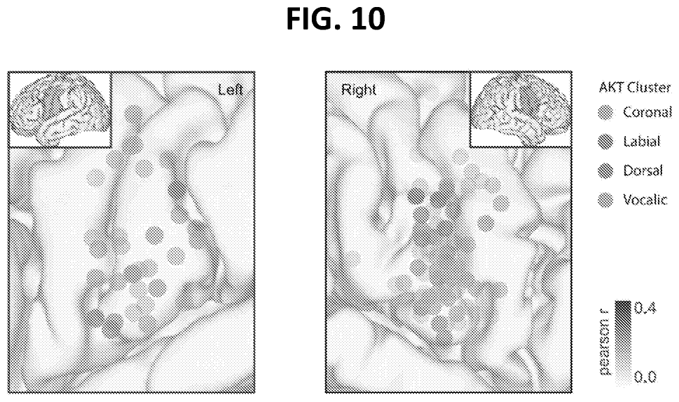

The anatomical clustering of AKTs was also examined across vSMC for each participant. While the anatomical clusterings for coronal and labial AKTs were significant (p <0.01, Wilcoxon signed-rank tests), clusterings for dorsal and vocalic AKTs were not. To further investigate the anatomical locations of AKT clusters, electrode locations were projected from all participants onto a common brain (FIG. 10). It was found that this coarse somatotopic organization was present for AKTs, which were spatially localized according to kinematic function and place of articulation. Since AKTs encoded coordinated articulatory movements, single articulator localization was not found. For example, with detailed descriptions of articulator movements, lower incisor movements were not localized to a single region; rather, opening and closing movements were represented separately, as seen in vocalic and coronal AKTs, respectively.

Damped Oscillatory Dynamics of Trajectories

Similar to motor cortical neurons involved in limb control, it was found that the encoded kinematic properties were time-varying trajectories. However, in contrast to the variety of trajectory patterns found during limb control from single neurons, it was observed that each AKT exhibited an out and-back trajectory from single ECoG electrode recordings. To further investigate the trajectory dynamics of every AKT, phase portraits (velocity and displacement relationships) were analyzed for each articulator. In FIG. 11A, the encoded position and velocity of trajectories of each articulator were shown, along its principal axis of displacement, for AKTs of four example electrodes, each representative of a main AKT cluster. The trajectory of each articulator was determined by the encoding weights from each AKT. All trajectories moved outward and then returned to the same position as the starting point with corresponding increases and decreases in velocity forming a loop. This was true even for articulators that only made relatively small movements.

In FIG. 11B, the trajectories for each articulator from all 108 AKTs were shown, which again illustrate the out-and-back trajectory patterns. Trajectories for a given articulator did not exhibit the same degree of displacement, indicating a level of specificity for AKTs within a particular cluster. Qualitatively, it was observed that trajectories with more displacement also tended to correspond with high velocities.

While each AKT specifies time-varying articulator movements, the governing dynamics dictating how each articulator moves may be time invariant. In articulator movement studies, the time-invariant properties of vocal tract gestures have been described by damped oscillatory dynamics Just like a pendulum, descriptors of movement (i.e., velocity and position) are related to one another independent of time. A linear relationship was found between peak velocity and displacement for every articulator described by the AKTs (FIG. 11C; r=0.85, 0.77, 0.83, 0.69, 0.79, and 0.83, in respective order; p<0.001), demonstrating that AKTs also exhibited damped oscillatory dynamics Furthermore, the slope associated with each articulator revealed the relative speed of that articulator. The lower incisor and upper lip moved the slowest (0.65 and 0.65 slopes), and the tongue varied in speed along the body, with the tip moving fastest (0.66, 0.78, and 0.99 slopes, respectively). These dynamics indicate that an AKT makes a stereotyped trajectory to form a single vocal tract configuration, a sub-syllabic speech component, acting as a building block for the multiple vocal tract configurations required to produce single syllables. The velocity- position relationship strongly indicates that the AKT model encoded movements for each articulator corresponding to the intrinsic dynamics of continuous speech production.

Coarticulated Kinematic Trajectories

Some of the patterns observed in the detailed kinematics of speech result from interactions between successive vocal tract constrictions, a phenomenon known as coarticulation. Depending on the kinematic constraints of vocal tract constrictions, some vocal tract constrictions may require anticipatory or carryover modifications to be optimally produced. Despite these modifications, each vocal tract constriction is often thought of as an invariant articulatory unit of speech production in which context-dependent kinematic variability results from the co-activation (i.e., temporal overlap) of vocal tract constrictions. At least some coarticulatory effects were found to arise from intrinsic biomechanical properties of the vocal tract. Whether the vSMC shared similar invariant properties by studying how vSMC representations of vocal tract AKTs interacted with one another during varying degrees of anticipatory and carryover coarticulation was investigated.

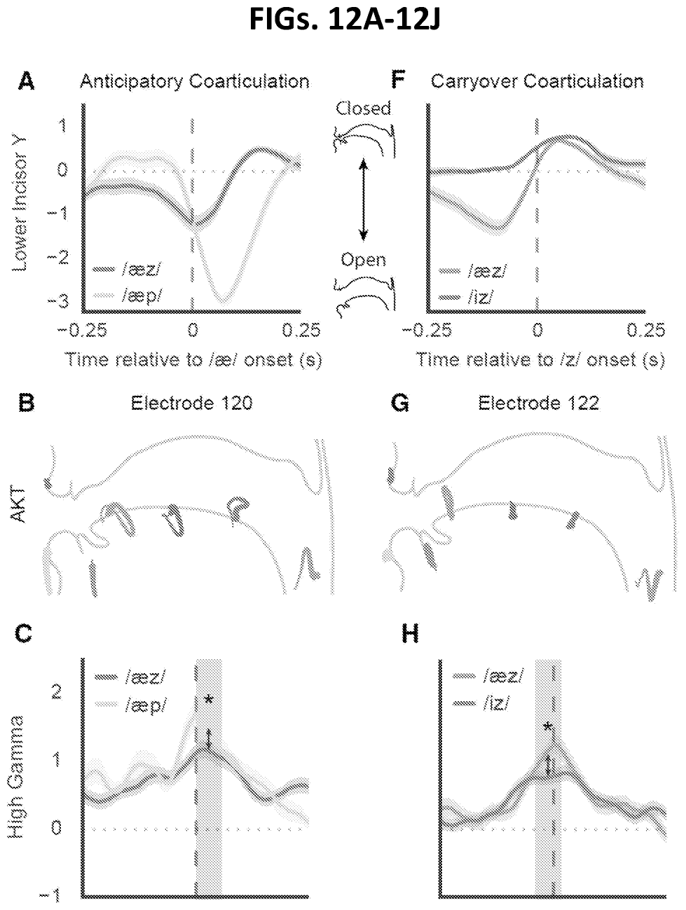

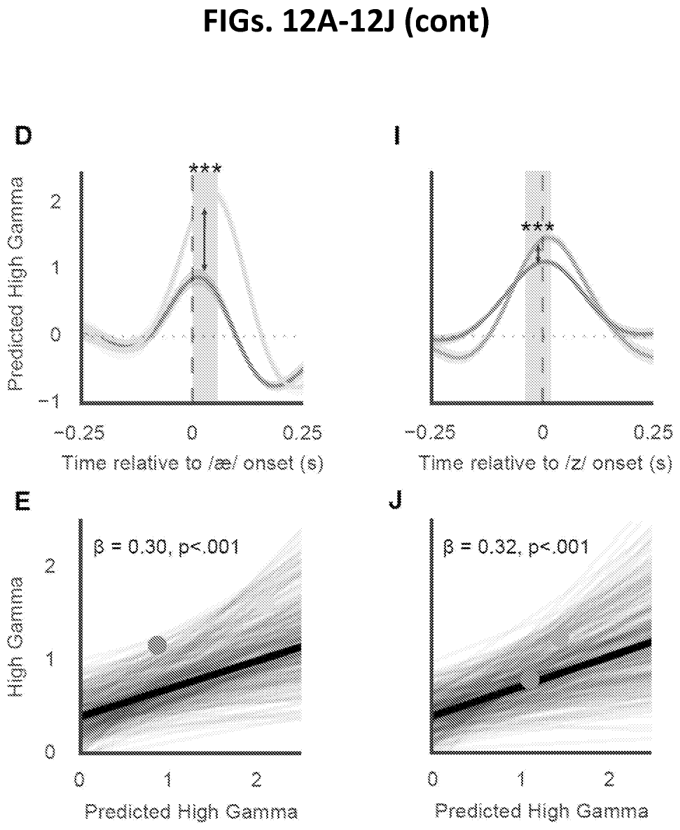

During anticipatory coarticulation, kinematic effects of upcoming phonemes may be observed during the production of the present phoneme. For example, consider the differences in jaw opening (lower incisor goes down) during the productions of /æz/ (as in “has”) and /æp/ (as in “tap”) (FIG. 12A). The production of /æ/ requires a jaw opening, but the degree of opening is modulated by the upcoming phoneme. Since /z/ requires a jaw closure to be produced, the jaw opens less during /æz/ to compensate for the requirements of /z/. On the other hand, /p/ does not require a jaw closure and the jaw opens more during /æp/. In each context, the jaw opens during /æ/, but to differing degrees based the compatibility of the upcoming movement.

To investigate whether anticipatory coarticulation is neurally represented, the change in neural activity was investigated during the production /æz/ and /æp/, two contexts with differing degrees of coarticulation. While vSMC activity at the electrode population level is biased toward surrounding contextual phonemes, the representation of coarticulation was investigated at single electrodes. High gamma of an electrode that encoded a vocalic AKT was studied, crucial for the production of /æ/ (high phonetic selectivity index for /æ/). In FIG. 12B, the AKT for electrode 120 describes a jaw opening and laryngeal vocal tract configuration. Time locked to the acoustic onset of /æ/, high gamma for electrode 120 was higher during /æp/ than /æz/ (FIG. 12C). To quantify this difference, the median high gamma activity was compared during 50 ms centered on the point of peak discriminability for all phonemes (p<0.05, Wilcoxon signed-rank tests). It was also found that the predicted high gamma from the AKT was similarly higher during /æp/ than /æz/ (p<0.001, Wilcoxon signed-rank tests) (FIG. 12D). For this electrode, it was found that high gamma activity reflected changes in kinematics, as predicted by the AKT, due to anticipatory coarticulation effects.

Whether coarticulatory effects were present in all vSMC electrodes during all the anticipatory contexts of every phoneme were examined To quantify this effect, a mixed-effects model was fit to study how high gamma for a given electrode changed during the production of a phoneme with different following phonemes.

In particular, for an electrode with an AKT heavily involved in producing a given phoneme, the kinematic compatibility of the following phoneme would be reflected in its peak high gamma The model used cross-random effects to control for differences across electrodes and phonemes and a fixed effect of predicted high gamma from the AKT to describe the kinematic variability to which each electrode is sensitive. In FIG. 12E, each line shows the relationship between high gamma and coarticulated kinematic variability for a given phoneme and electrode in all following phonetic contexts with at least 25 instances. For example, one line indicates how high gamma varied with the kinematic differences during /tæ/, /ta/, . . . , /ts/, etc. Kinematic variability due to following phonemes was a significant effect of the model indicating that neural activity associated with particular articulatory movements is modulated by the kinematic constraints of the following articulatory context (b=0.30, SE=0.04, c2(1)=38.96, p=4e-10).

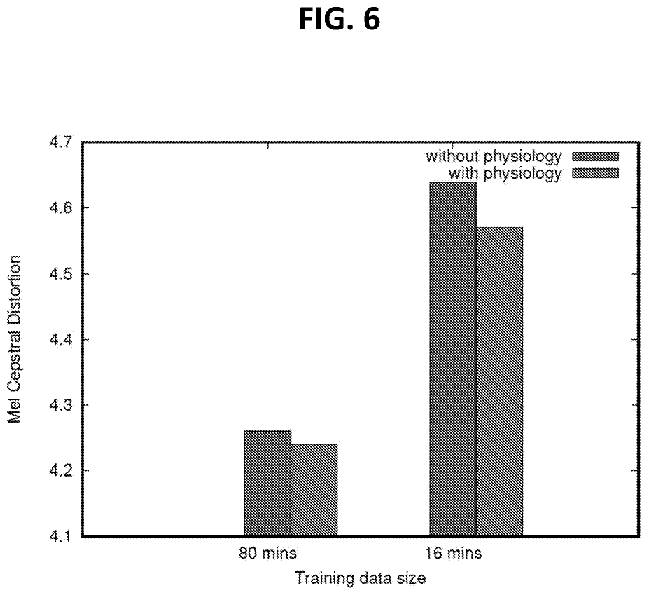

In a similar fashion, the neural representation of carryover articulation was also investigated, in which kinematic effects of previously produced phonemes are observed. In FIG. 12F, two coarticulated contexts with varying degrees of compatibility were shown: /æz/ (as in “has”) and /iz/ (as in “ease”). /æ/ involves a large jaw opening while /i/ does not. However, in both contexts the jaw is equally closed for /z/ and the major difference between /æz/ and /iz/ is how much the jaw must move to make the closure. While the target jaw position for /z/ was achieved in both contexts, it was found that for an electrode with a coronal AKT involved in producing /z/ (FIG. 12G), the difference in high gamma reflected the kinematic differences between the two preceding phonemes (FIGS. 6H and 6I). Again, a mixed effects model was used to examine the effects of carryover coarticulation in all vSMC electrodes to find that neural activity reflected carried-over kinematic differences in electrodes with AKTs for making the present phoneme (b=0.32, SE=0.04, c2(1)=42.58, p=6e-11) (FIG. 12J). These results indicate that electrodes involved in producing a particular vocal tract configuration reflect kinematic variability due to anticipatory and carryover coarticulation.

Comparison with Other Encoding Models

To evaluate how well AKTs are encoded in the vSMC, the following were compared: (1) the AKT model's encoding performance with respect to other cortical regions and (2) vSMC-encoding models for alternative representations of speech.

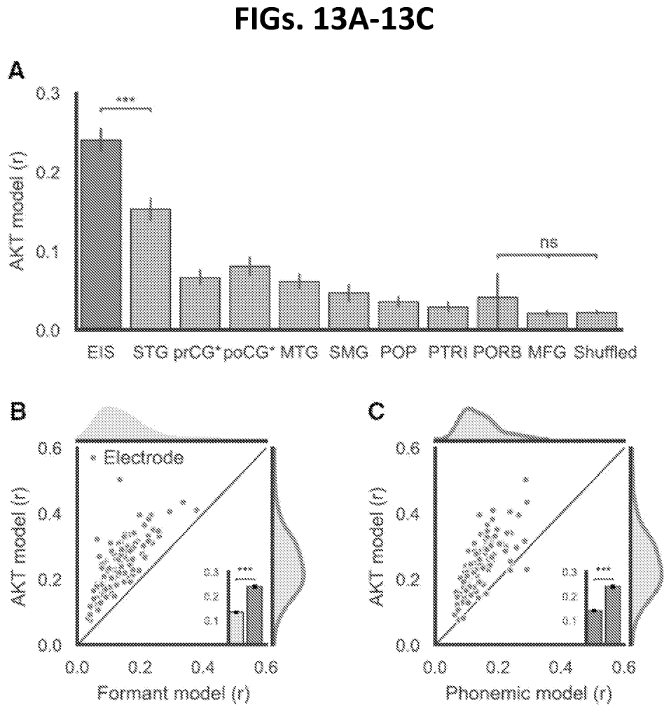

To determine how specific AKTs are to the vSMC, AKT model performance (Pearson's r on held-out data) of every cortical region recorded from across participants (FIG. 13A) was compared. Besides electrodes from middle frontal gyrus (MFG) and pars orbitalis (n=4), the AKT model significantly explained some of the variance for all recorded cortical regions above chance level (p<0.001, Wilcoxon rank-sum test). However, for the considered electrodes in this study (EIS)—i.e., the speech active electrodes in the vSMC—the AKT model explained neural activity markedly better than in other cortical areas (p<1e-15, Wilcoxon rank-sum test). The other cortical areas that were examined were shown to be involved in different aspects of speech processing—acoustic and phonological processing (superior temporal gyrus [STG] and middle temporal gyms [MTG]) and articulatory planning (inferior frontal gyrus [IFG]). Therefore, it was expected that cortical activity in these regions would have some correlation to the produced kinematics. The higher performance of the AKT model for EIS indicates that studying the neural correlates of kinematics may best focused in the vSMC.

While AKTs were best encoded in vSMC, there may be alternative representations of speech that may better explain vSMC activity. vSMC encoding of both acoustics (described here by using the first three formants: F1, F2, and F3) and phonemes were evaluated with respect to the AKT model. Each model was fit in the same manner as the AKT model and performance compared on held-out data from training. If each vSMC electrode represented acoustics or phonemes, then a higher model fit was expected for that representation than the AKT model. Due to the similarity of these representations, the encoding models were expected to be highly correlated. It is worth noting that the inferred articulator movements are unable to provide an account of movements without correlations to acoustically significant events, a key property that would be invaluable for differentiating between models.

Furthermore, while acoustics and phonemes are both complete representations of speech, the midsagittal movements of a few vocal tract locations captured by EMA are a partial description of speech relevant movements of the vocal tract in that there are missing palate, lateral, and oropharyngeal movements. Even so, it was found that articulator movements were encoded markedly better than both the acoustic and phoneme-encoding models despite the limitations of the AKT model (FIGS. 7B and 7C; p<1e-20, Wilcoxon rank-sum test).

Therefore, vSMC encoding is tuned to articulatory features. During single-vowel production, the vSMC showed encoding of directly measured kinematics over phonemes and acoustics.

Furthermore, the vSMC is also responsible for non-speech voluntary movements of the lips, tongue, and jaw in behaviors such as swallowing, kissing, and oral gestures. While vSMC is critical for speech production, it is not the only vSMC function. Indeed, when the vSMC is injured, patients have facial and tongue weakness, in addition to dysarthria. When the vSMC is electrically stimulated, movements were observed, but not speech sounds, phonemes, or auditory sensations.

Decoding Articulator Movements

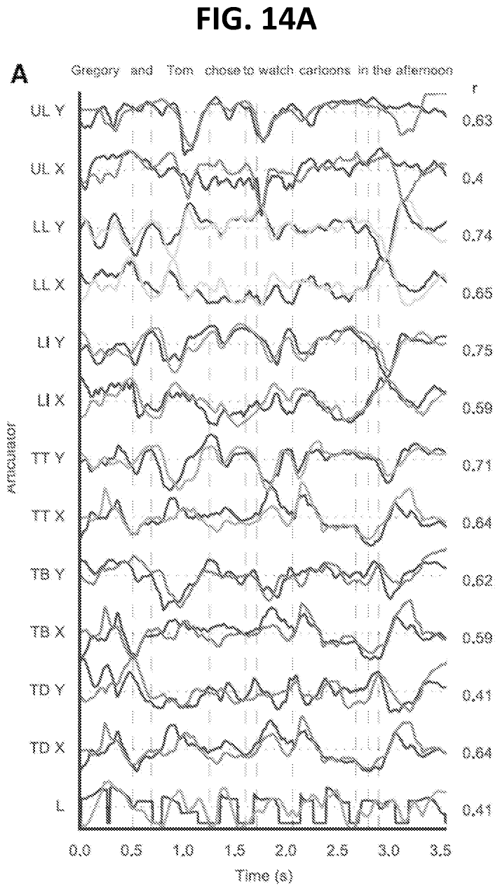

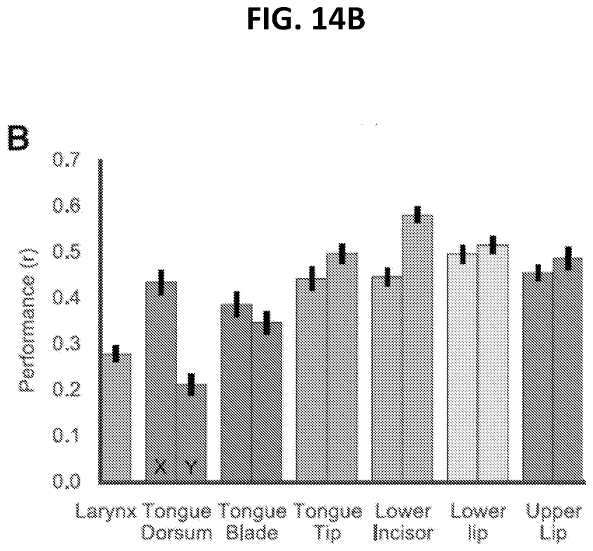

Given that encoding of AKTs at single electrodes could be determined, understanding how well decoding vocal tract movements from the population of electrodes was studied. Articulatory movements were decoded during sentence production with a long short-term memory recurrent neural network (LSTM), an algorithm well suited for time- series regression. The performance of the decoder was high, especially in light of the articulatory variance lost due to process of inferring kinematics and the neural variance unrecorded by the ECoG grid (i.e., within the central sulcus or at a resolution finer than the capability of the electrodes). For an example sentence (FIG. 14A), the predicted articulator movements from the decoder closely matched with the inferred articulator movements from the acoustics. All of the articulator movements were well predicted across 100 held-out sentences significantly above chance (mean r=0.43, p<0.001) (FIG. 14B).

### Discussion

A novel AAI method was used to infer vocal tract movements, which was then related directly to high-resolution neural recordings. By describing vSMC activity with respect to detailed articulatory movements, it was demonstrated that discrete neural populations encode AKTs.

As provided herein, features of the AKTs that are encoded in the vSMC are shown. First, encoded articulator movements are coordinated to make a specific vocal-tract configuration.

vSMC activity was studied using detailed articulatory trajectories that suggest that similar to limb control, coordinated movements across articulators for specialized vocal tract configurations are encoded at the single electrode level. For example, the coordinated movement to close the lips is encoded rather than individual lip movements.

For speech, four major clusters of AKTs were found that were differentiated by place of articulation and covered the main vocal tract configurations that comprise American English. At the sampling level of ECoG, cortical populations encode sub-syllabic coordinative movements of the vocal tract.

As provide herein another feature of AKTs includes the trajectory profile itself. Encoded articulators moved in out-and-back trajectories with damped oscillatory dynamics During limb control, single motor cortical neurons have been also found to encode time-dependent kinematic trajectories, but the patterns were very heterogeneous and did not show clear spatial organization. It is possible that individual neurons encode highly specific movement fragments that combine to form larger movements represented by ensemble activity at the ECoG scale of resolution.

For speech, these larger movements correspond to canonical vocal tract configurations. While motor cortical neurons encoded a variety of trajectory patterns, AKTs was found to only exhibited out and- back profiles that may be a fundamental movement motif in continuous speech production.

With both coordinative and dynamical properties, each AKT appeared to encode the movement necessary to make a specific vocal tract configuration and return to a neutral position. Each vocal tract gesture is described as coordinated articulatory pattern to make a vocal tract constriction. Like the AKTs, each vocal tract gesture has been characterized as a time-invariant system with damped oscillatory dynamics

AKTs encoded in vSMC neural activity were found to be reflected kinematic differences due to constraints of the phonetic or articulatory context.

### Experimental Model and Subject Details

### Participants

Five human participants (Female, ages: 30, 31, 43, 46, 47) underwent chronic implantation of high-density, subdural electrode array over the lateral surface of the brain as part of their clinical treatment of epilepsy (2 left hemisphere grids, 3 right hemisphere grids).

Participants gave their written informed consent before the day of the surgery. No participants had a history of any cognitive deficits that were relevant to the aims of the present study. All participants were fluent in English. All procedures were approved by the University of California, San Francisco Institutional Review Board.

### Method Details

### Experimental Task

Participants read aloud 460 sentences from the MOCHA-TIMIT database. Sentences were recorded in 9 blocks (8 of 50, and 1 of 60 sentences) spread across several days of patients' stay. Within each block, sentences are presented on a screen, one at a time, for the participant to read out. The order was random and participants were given a few seconds of rest in between.

MOCHA-TIMIT is a sentence-level database, a subset of the TIMIT corpus designed to cover all phonetic contexts in American English. Each participant read each sentence 1-10 times. Microphone recordings were obtained synchronously with the ECoG recordings.

### Data Acquisition and Signal Processing

Electrocorticography was recorded with a multi-channel amplifier optically connected to a digital signal processor. Speech was amplified digitally and recorded with a microphone simultaneously with the cortical recordings. ECoC electrodes were arranged in a 16 3 16 grid with 4 mm pitch. The grid placements were decided upon purely by clinical considerations. ECoG signals were recorded at a sampling rate of 3,052 Hz. Each channel was visually and quantitatively inspected for artifacts or excessive noise (typically 60 Hz line noise). The analytic amplitude of the high-gamma frequency component of the local field potentials (70-150 Hz) was extracted with the Hilbert transform and down-sampled to 200 Hz. Finally, the signal was z-scored relative to a 30 s window of running mean and standard deviation, so as to normalize the data across different recording sessions. High-gamma amplitude was studied because it correlates well with multi-unit firing rates and has the temporal resolution to resolve fine articulatory movements.

### Phonetic and Phonological Transcription

For the collected speech acoustic recordings, transcriptions were corrected manually at the word level so that the transcript reflected the vocalization that the participant actually produced. Given sentence level transcriptions and acoustic utterances chunked at the sentence level, hidden Markov model based acoustic models were built for each participant so as to perform sub-phonetic alignment. Phonological context features were also generated from the phonetic labels, given their phonetic, syllabic and word contexts.

### Speaker-Independent Acoustic-to-Articulatory Inversion (AAI)

To perform articulatory inversion for a target participant for whom only acoustic data is available, a method was developed, referred to herein as “Speaker-Independent AAI,” where parallel EMA and speech data were simulated for the target speaker. In contrast to earlier approaches for speaker-independent AAI, where normalization is performed to remove speaker identity from acoustics, the opposite goal was accomplished of transforming the 8 EMA participants' spectral properties to match those of the target speaker for whom an estimate vocal tract kinematics was wanted. To transform the acoustics of all data to the target speaker, a voice conversion was applied to transform the spectral properties of each EMA speaker to match those of the target participant. This method assumes acoustic data corresponding to the same sentences for the two participants. When parallel acoustic data was not available across participants (the mngu0 corpus uses a different set of sentences than the MOCHA-TIMIT corpus), concatenative speech synthesis were used to synthesize comparable data across participants.

For cross-participant utilization of kinematic data, for each of the training speakers, an articulator specific z-scoring across each participant's EMA data was used. This ensured that the target speaker's kinematics were an unbiased average across all available EMA participants. The kinematics were described by 13 dimensional feature vectors (12 dimensions to represent X and Y coordinates of 6 vocal tract points and fundamental frequency, F0, representing the Laryngeal function).

24 dimensional mel-cepstral coefficients were used as the spectral features. Both kinematics and acoustics were sampled at a frequency 200 Hz (each feature vector represented a 5 ms segment of speech). Additionally, phonetic and phonological information corresponding to each frame of speech was coded as one-hot vectors and padded onto the acoustic features. These features included phoneme identity, syllable position, word part of speech, positional features of the current and of the neighboring phoneme and syllable states. Contextual data was found to provide complementary information to acoustics and improved inversion accuracies.

Using these methods for each EMA participant-to-target participant pair, a simulated dataset of parallel speech and EMA data was created, that were both customized for the target participant. For training the inversion model itself, a deep recurrent neural network was used based articulatory inversion technique to learn a mapping from spectral and phonological context to a speaker generic articulatory space. An optimal network architecture with a 4 layer deep recurrent network with two feedforward layers (200 hidden nodes) and two bidirectional LSTM layers (with 100 LSTM cells) was chosen. The trained inversion model was then applied to all speech produced by the target participant to infer articulatory kinematics in the form of Cartesian X and Y coordinates of articulator movements. The network was implemented using Keras, a deep learning library running on top of a Tensorflow backend.

### Electrode Selection

Electrodes located on either the precentral and postcentral gyri that had distinguishable high gamma activity during speech production were selected. The separability of phonemes was measured using the ratio of between-class to within-class variability (F statistic) for a given electrode across time. Electrodes with a maximum F statistic of 8 or greater were chosen. This resulted in a total of 108 electrodes across the 5 participants with robust activity during speech production.

### Encoding Models

To uncover the kinematic trajectories represented in electrodes, linear encoding models were used to describe the high gamma activity recorded at each electrode as a weighted sum of articulator kinematics over time. This model is similar to the spectrotemporal receptive field, a model widely used to describe selectivity for natural acoustic stimuli. However, in the model presented herein, articulator X and Y coordinates are used instead spectral components. The model estimates the time series xi(t) for each electrode i as the convolution of the articulator kinematics A, comprised of kinematic parameters k, and a filter H, which is referred to as the articulatory kinematic trajectory (AKT) encoding of an electrode.

$${\hat{x}}_{i} ( t ) = {\sum }_{k}^{K} {\sum }_{τ}^{T} {H}_{i} ( k , τ ) A ( k , t - τ )$$

The filter provided herein is designed to use a 500 ms window of articulator movements centered about the high gamma sample to be predicted. Movements occurring

before the sample of high gamma are indicated by a negative lag while movements occurring after the high gamma sample are indicated by a positive lag. The 500 ms window was chosen to both maximize the performance of the AKT model (Figure S6) and allow full visualization of the AKTs. While Figure S6, indicates the filters need only be 200 ms long for optimal performance, it was found that extending filters to 500 ms with appropriate regularization ensured that visualization occured every AKT in its entirety. Some AKTs encoded movements occurring well before or after the corresponding neural activity resulting AKTs cutoff using a 200 ms window. L2 regularization ensured that weights from time points not encoding an articulatory trajectory (e.g., at 250 ms before the neural sample) had no weighting and did not affect interpretability of the AKTs.

Additionally, acoustic and phoneme encoding models were fit to electrode activity. Instead of articulator X and Y coordinates, formants (F1, F2, and F3) were used as a description of acoustics and a binary description of the phonemes produced during a sentence. Each feature indicated whether a particular phoneme was being produced or not with a 1 or 0, respectively. The encoding models were fit using ridge regression and trained using cross-validation with 70% of the data used for training, 10% of the data held-out for estimating the ridge parameter, and 20% held out as a final test set. The final test set consisted of sentences produced during entirely separate recording sessions from the training sentences. Performance was measured as the correlation between the predicted response of the model and the actual high gamma measured in the final test set.

### Hierarchical Clustering

Ward's method was used for agglomerative hierarchical clustering. Clustering of the electrodes was carried out solely on the kinematic descriptions for encoded kinematic trajectory of each electrode. To develop concise kinematic descriptions for each kinematic trajectory, the point of maximal displacement was extracted for each articulation. Principal components analysis was used on each articulator to extract the direction of each articulator that explained the most variance. The filter weights were then projected onto each articulator's first principal component and chose the point with the highest magnitude. This resulted in length 7 vector with each articulator described by the maximum value of the first principal component. Phonemes were clustered based on the phoneme encoding weights for each electrode.

For a given electrode, the maximum encoding weight was extracted for each phoneme during a 100 ms window centered at the point of maximum phoneme discriminability (peak F statistic) for the given electrode.

### Cortical Surface Extraction and Electrode Vsualization

To visualize electrodes on the cortical surface of a participant's brain, a normalized mutual information routine was used in SPM12 to co-register the preoperative T1 MRI with a postoperative CT scan containing electrode locations. Freesurfer was used to make pial surface reconstructions. To visualize electrodes across participants on a common MNI brain, nonlinear surface registration was performed using a spherical sulcal-based alignment in Freesurfer, aligned to the cvs avg35 inMNI152 template. While the geometry of the grid is not maintained, the nonlinear alignment ensures that electrodes on a gyrus in the participant's native space will remain on the same gyrus in the atlas space.

### Decoding Model

To decode articulatory movements, a long short-term memory (LSTM) recurrent neural network was trained to learn the mapping from high gamma activity to articulatory movements. LSTM are particularly well suited for learning mappings with time-dependent information. Each sample of articulator position was predicted by the LSTM using a window of 500 ms of high gamma activity, centered about the decoded sample, from all vSMC electrodes. The decoder architecture was a 4 layer deep recurrent network with two feedforward layers (100 hidden nodes each) and two bidirectional LSTM layers (100 cells).

Using Adam optimization and dropout (40% of nodes), the network was trained to reduce mean squared error of the decoded and actual output. The network was implemented using Keras, a deep learning library running on top of a Tensorflow backend.

### Quantification and Statistical Analysis

### Nested Encoding Model Comparison

A nested regression model was used to compare the neural encoding of a single articulator trajectory with the AKT model. For each electrode, single articulatory trajectories models were fit using both X and Y directions for each EMA sensor and chose the single articulator model that with the lowest residual sum of squares (RSS) on held-out data. From RSS values for the full (2) and nested (1) models, the significance of the explained variance was compared by calculating an F statistic for each electrode.

$$F = \frac{( \frac{{RSS}_{1} - {RSS}_{2}}{{p}_{2} - {p}_{1}} )}{\frac{{RSS}_{2}}{n - {p}_{2}}}$$

p and n are the number of model parameters and samples used in RSS computation, respectively. An F statistic greater than the critical value defined by the number of parameters in both models and confidence interval indicates that the full model (AKT) explains statistically significantly explains more variance than the nested model (single articulator) after accounting for difference in parameter numbers.

### Correlation Structure Comparison

To test whether the correlational structure of articulators (EMA points) was different between periods of low and high gamma activity for a speech responsive electrode, the inferred articulator movements was split into two datasets based on whether the z-scored high gamma activity of given electrode for that sample was above the threshold (1.5). 1000 points of articulator movement was then randomly sampled from each dataset to construct two cross-correlational structures between articulators. To quantify the difference between the correlational structures, the Euclidean distance was computed between the two structures. An additional 1000 points were then sampled from the below threshold dataset to quantify the difference between correlational structures within the sub-threshold data. This process was repeated 1000 times for each electrode and compared the two distributions of Euclidean distances with a Wilcoxon rank sum test (Bonferroni corrected for multiple comparisons) to determine whether correlational structures of articulators differed in relation to high or low high gamma activity of an electrode.

### Silhouette Analysis

To assess cluster separability, the silhouette index was computed for each electrode to compare how well each electrode matched its own cluster based on the given feature representation. The silhouette index for an electrode is calculated by taking the difference between the average dissimilarity with all electrodes within the same cluster and the average dissimilarity with electrodes from the nearest cluster. This value is then normalized by taking the maximum value of the previous two dissimilarity measures. A silhouette index close to 1 indicates that the electrode is highly matched to its own cluster. 0 indicates that that the clusters may be overlapping, while −1 indicates that the electrode may be assigned to the wrong cluster.

Phoneme Selectivity Index (PSI)

To determine the phoneme selectivity of each electrode, the statistical framework was used to test whether the high gamma activity of an electrode is significantly different during the productions of two different phonemes. For a phoneme pair and a given electrode, two distributions of high gamma activity were created from data acoustically aligned to each phoneme. A 50 ms window of activity centered on the time point was used with the peak F statistic for that electrode. A non-parametric statistical hypothesis test (Wilcox rank-sum test) was used to assess whether these distributions have different medians (p<0.001). The PSI is the number of phonemes that have statistically distinguishable high gamma activity for a given electrode.

A PSI of 0 indicates that no other phonemes have a distinguishable high gamma activity. Whereas, a PSI of 40 indicates that all other phonemes have distinguishable high gamma activity.

### Mixed Effects Model

To examine the relationship between high gamma and coarticulated kinematics, a mixed-effects model was used with several crossed random effects. In particular, for a given electrode, the “peak activity” was computed by taking the median high gamma activity during a 50 ms window centered about the peak F statistic for that electrode (see PSI method) during the production of a target phoneme.

The mean peak activity was then taken for each unique phoneme pair (target phoneme preceded by context phoneme). For each electrode, phoneme pairs with at least 25 instances were considered and a target PSI>25. This helped stabilize the means and targeted electrodes that presumably encoded the AKT necessary to produce the target phoneme. In FIGS. 6C, 6D, 6H, and 6I, /z/ was extended to include /z/ and /s/, and /p/ to include /p/ and /b/ since, from an EMA standpoint, the articulation is nearly identical and it increased the number of coarticulated instances that could be analyzed, thus decreasing biases from other contextual effects and variability from noise. In a similar fashion to high gamma, high gamma activity predicted by the AKT model was computed to provide insight into the kinematics during the production of a particular phoneme pair. The mixed-effects model described high gamma from a fixed effect of kinematically predicted high gamma with crossed random effects (random slopes and intercepts) controlling for difference in electrodes, and target and context phonemes. To determine model goodness, ANOVA was used to compare the model with a nested model that retained the crossed random effects but removed the fixed effect. The mixed-effects model was fit using the lme4 package in R.

### Example 3

### Speech Synthesis from Neural Decoding of Spoken Sentences

A neural decoder was designed that explicitly leverages kinematic and sound representations encoded in human cortical activity to synthesize audible speech. Recurrent neural networks first decoded directly recorded cortical activity into articulatory movement representations, and then transformed those representations into speech acoustics. In closed vocabulary tests, listeners could readily identify and transcribe neurally synthesized speech. Intermediate articulatory dynamics enhanced performance even with limited data. Decoded articulatory representations were highly conserved across speakers, enabling a component of the decoder be transferrable across participants. Furthermore, the decoder could synthesize speech when a participant silently mimed sentences. These findings advance the clinical viability of speech neuroprosthetic technology to restore spoken communication.

A biomimetic approach that focuses on vocal tract movements and the sounds they produce can achieve the high communication rates of natural speech, and also likely the most intuitive for users to learn. In patients with paralysis, for example from ALS or brainstem stroke, high fidelity speech control signals may only be accessed by directly recording from intact cortical networks.

The feasibility of a neural speech prosthetic was demonstrated by translating brain signals into intelligible synthesized speech at the rate of a fluent speaker. High-density electrocorticography (ECoG) signals were recorded from five participants undergoing intracranial monitoring for epilepsy treatment as they spoke several hundred sentences aloud. A recurrent neural network was designed that decoded cortical signals with an explicit intermediate representation of the articulatory dynamics to synthesize audible speech.

### Speech Decoder Design

The two-stage decoder approach is shown in FIG. 15A-15D. Stage 1: a bidirectional long short-term memory (bLSTM) recurrent neural network decodes articulatory kinematic features from continuous neural activity (high-gamma amplitude envelope and low frequency component) recorded from ventral sensorimotor cortex (vSMC), superior temporal gyms (STG), and inferior frontal gyrus (IFG) (FIG. 15A-15B). Stage 2: a separate bLSTM decodes acoustic features (F₀, mel-frequency cepstral coefficients (MFCCs), voicing and glottal excitation strengths) from the decoded articulatory features from Stage 1 (FIG. 1C). The audio signal is then synthesized from the decoded acoustic features (FIG. 15D). To integrate the two stages of the decoder, Stage 2 (articulation-to-acoustics) was trained directly on output of Stage 1 (brain-to-articulation) so that it not only learns the transformation from kinematics to sound, but can correct articulatory estimation errors made in Stage 1.

A component of the decoder of the present disclosure is the intermediate articulatory representation between neural activity and acoustics (FIG. 15B). The vSMC exhibits robust neural activations during speech production that predominantly encode articulatory kinematics. A statistical approach was used to estimate vocal tract kinematic trajectories (movements of the lips, tongue, and jaw) and other physiological features (e.g. manner of articulation) from audio recordings. These features initialized the bottleneck layer within a speech encoder-decoder that was trained to reconstruct a participant's produced speech acoustics. The encoder was then used to infer the intermediate articulatory representation used to train the neural decoder. With this decoding strategy, it was possible to accurately reconstruct the speech spectrogram.

### Synthesis Performance

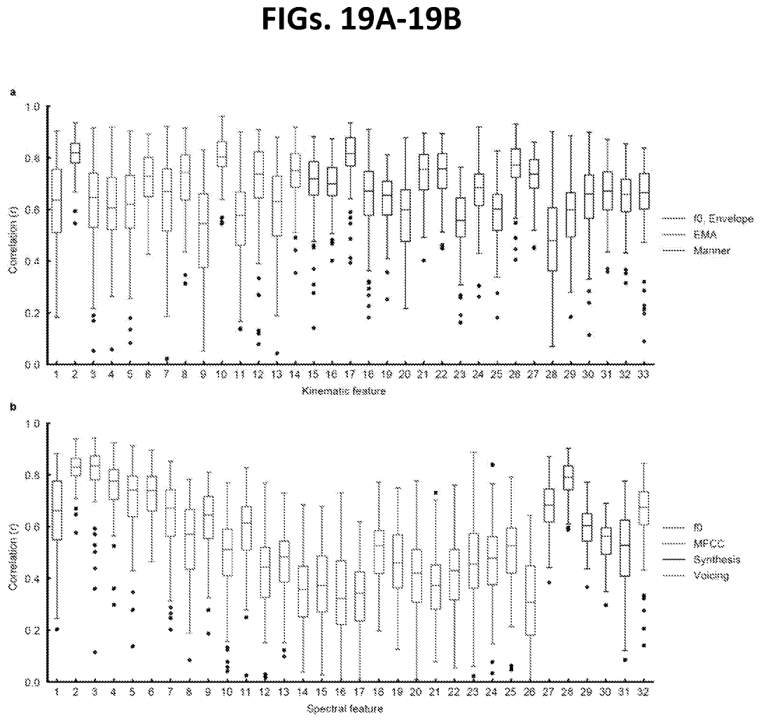

Overall, detailed reconstructions of speech synthesized from neural activity alone was observed. FIGS. 15E-15F shows the audio spectrograms from two original spoken sentences plotted above those decoded from brain activity. The decoded spectrogram retained salient energy patterns present in the original spectrogram and correctly reconstructed the silence in between the sentences when the participant was not speaking. FIGS. 19A-19B, illustrates the quality of reconstruction at the phonetic level. Median spectrograms of original and synthesized phonemes showed that the typical spectrotemporal patterns were preserved in the decoded exemplars (e.g. formants F1-F3 in vowels /i:/ and /æ/; and key spectral patterns of mid-band energy and broadband burst for consonants /z/ and /p/, respectively).

To understand to what degree the synthesized speech was perceptually inelligible to naïve listeners, two listening tasks were conducted that involved single-word identification and sentence-level transcription, respectively. The tasks were run on Amazon Mechanical Turk, using all 101 synthesized sentences from the test set for participant P1.

For the single-word identification task, 325 words were evaluated that were spliced from the synthesized sentences. The effect of word length (number of syllables) and the number of choices (10, 25, and 50 words) on speech intelligibility were quantified, since these factors inform optimal design of speech interfaces. It was found that listeners were more successful at word identification as syllable length increased, and number of word choices decreased, consistent with natural speech perception.

For sentence-level intelligibility, a closed vocabulary, free transcription task was designed. Listeners heard the entire synthesized sentence and transcribed what they heard by selecting words from a defined pool (of either 25 or 50 words) that included the target words and random words from the test set. The closed vocabulary setting was necessary because the test set was a subset of sentences from MOCHA-TIMIT which was primarily designed to optimize articulatory coverage of English but contains highly unpredictable sentence constructions and low frequency words.

Listeners were able to transcribe synthesized speech well. Of the 101 synthesized trials, at least one listener was able to provide a perfect transcription for 82 sentences with a 25-word pool and 60 sentences with a 50-word pool. Of all submitted responses, listeners transcribed 43% and 21% of the total trials perfectly, respectively (FIG. 6). Transcribed sentences had a median 31% WER with a 25-word pool size and 53% WER with a 50-word pool size. Table 1 shows listener transcriptions for a range of WERs. Median level transcriptions still provided a fairly accurate, and in some cases legitimate transcription (eg., “mum” transcribed as “mom” etc.). The errors suggest that the acoustic phonetic properties of the phonemes are still present in the synthesized speech, albeit to the lesser degree (eg., “rabbits” transcribed as “rodents”). This level of intelligibility for neurally synthesized speech would already be immediately meaningful and practical for real world application.

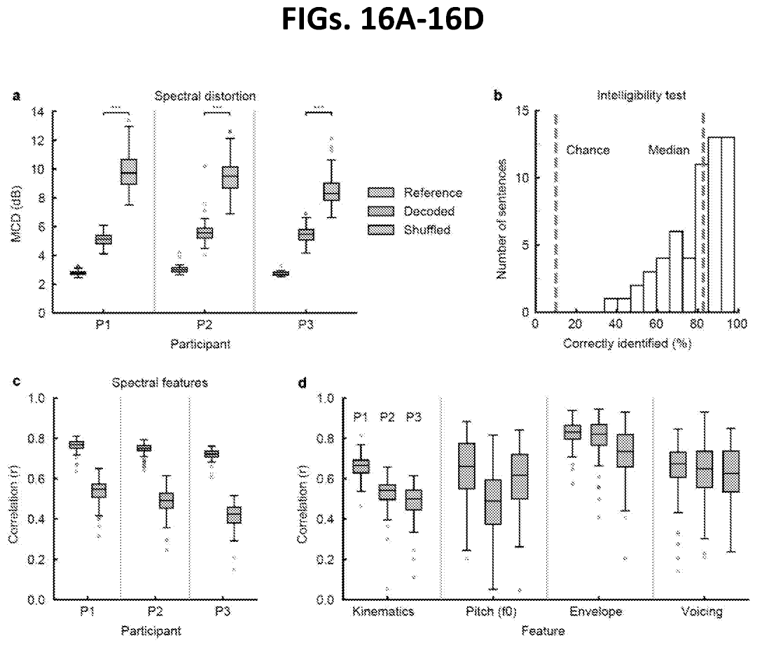

The decoding performance was then quantified at a feature level for all participants. In speech synthesis, the spectral distortion of synthesized speech from ground-truth is commonly reported using the mean Mel-Cepstral Distortion (MCD). Mel-Frequency bands emphasize the distortion of perceptually relevant frequency bands of the audio spectrogram. In FIG. 16A, the MCD of neurally synthesized speech was compared to a reference synthesis from articulatory kinematics and chance-level decoding (lower MCD is better). The reference synthesis acts as a bound for performance as it simulated what perfect neural coding of the kinematics would achieve. For the five participants (P1-5), the median MCD scores of decoding speech ranged from 5.14 dB, 5.55 dB, and 5.49 dB, all better than chance-level decoding (p<1e-18, n=100 sentences, Wilcoxon signed-rank test (WSRT), for each participant).

The correlations between original and decoded acoustic features were computed. For each sentence and feature, the Pearson's correlation coefficient was computed using every sample (at 200 Hz) for that feature. The sentence correlation of the mean decoded acoustic features (intensity+MFCCs+excitation strengths+voicing) and inferred kinematics across participants are plotted. Prosodic features such as pitch (F0), speech envelope, and voicing were decoded well above chance-level (r>0.6, except F0 for P2: r=0.49 and all features for P5, p<1e-10, WSRT, for all participants and features).

To assess perceptual intelligibility of the decoded speech, Amazon Mechanical Turk was used to evaluate naiive listener's ability to understand the neurally decoded trials. 166 people were asked to identify 10 sentences (written on screen) corresponded to the decoded audio they heard. The median percentage of participants who correctly identified each sentence was 83%, significantly above chance (10%) (FIG. 16B).

In addition to spectral distortion and intelligibility, the correlations between original and decoded spectral features were also examined The median correlations (of sentences, Pearson's r) of the mean decoded spectral feature (pitch+25 MFCCs+excitation strengths+voicing) for each participant were 0.55, 0.49, and 0.42 (FIG. 16C).

Similarly, for decoded kinematics (the intermediate representation), the median correlations were 0.66, 0.54, and 0.50 (FIG. 16D). Finally, three key aspects of prosody were examined for intelligible speech: pitch (f0), speech envelope, and voicing (FIG. 16D). For all participants, these features were decoded well above chance-level correlations (r>0.6, except f0 for P2: r=0.49, p<1e-10, n=100, WSRT, for all participants and features in FIGS. 16C-16D). Correlation decoding performance for all other features is shown in FIGS. 19A-19B.

### Effects of Model Design Decisions

The following analyses were performed on data from P1. In designing a neural decoder for clinical applications, there are several key considerations that determine model performance First, in patients with severe paralysis or limited speech ability, training data may be very difficult to obtain. Therefore, the amount of data necessary was assessed to achieve a high level of performance. A clear advantage was found in explicitly modeling articulatory kinematics as an intermediate step over decoding acoustics directly from the ECoG signals. The motivation for including articulatory kinematics was to reduce the complexity of the ECoG-to-acoustic mapping because it captures the physiological process by which speech is generated and is encoded in the vSMC. The “direct” decoder was a bLSTM recurrent neural network optimized for decoding acoustics (MFCCs) directly from same ECoG signals as employed in articulatory decoder.

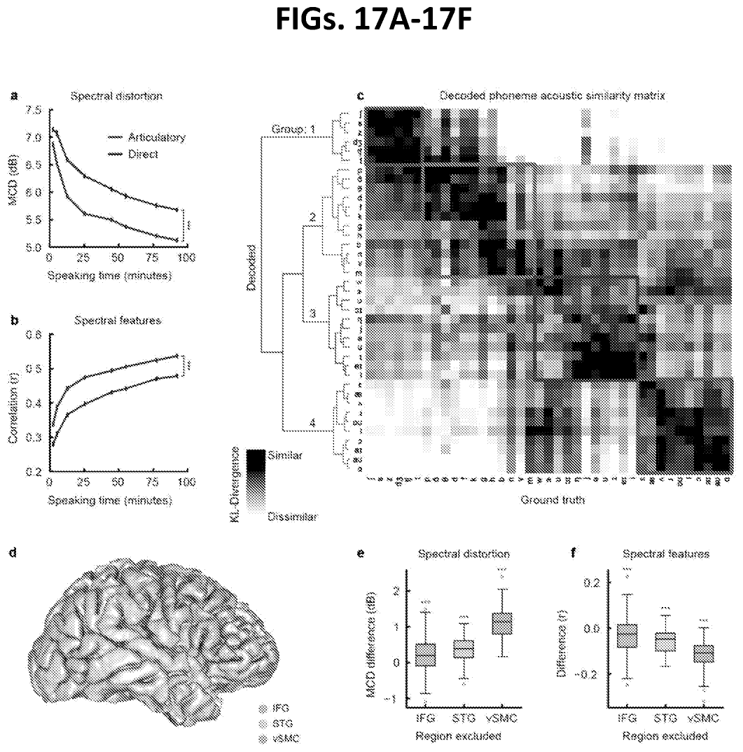

It was found that a robust performance could be achieved with as little as 25 minutes of speech, but performance continued to improve with the addition of data (FIG. 17A-17B). Without the articulatory intermediate step, the direct ECoG to acoustic decoding MCD was offset by 0.54 dB (0.2 dB is perceptually noticeable) using the full data set (FIG. 17A) (p=1e⁻¹⁷, n=101, WSRT), a substantial difference given that a change in MCD as small as 0.2 dB is perceptually noticeable The biomimetic approach using an intermediate articulatory representation requires less training data.

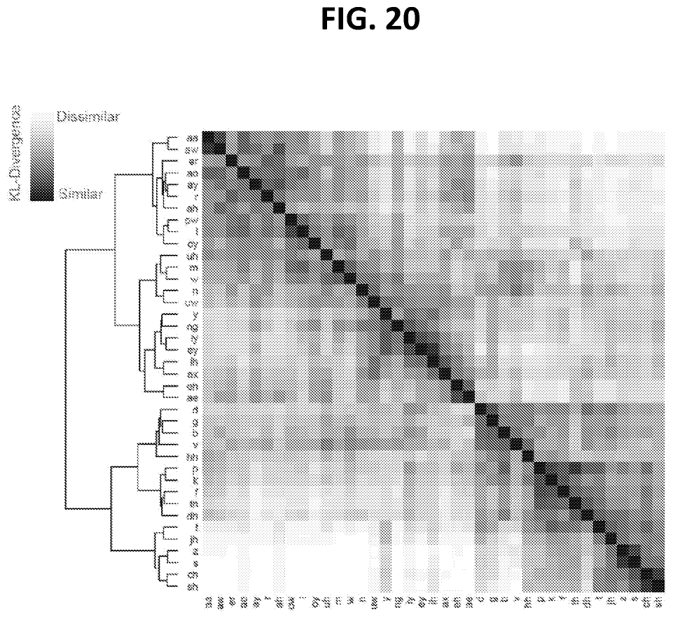

To understand the acoustic-phonetic properties that were preserved in decoded speech important for relative phonetic discrimination, the distribution of spectral features of each decoded phoneme to those of each ground-truth was compared by constructing a statistical distribution of the spectral feature vectors for each phoneme. Uinsg the Kullback-Leibler (KL) divergence, the distribution of each decoded phoneme was compared to the distribution of each ground-truth phoneme to determine how similar they were (FIG. 17C). From the acoustic similarity matrix of only ground-truth phoneme pairs (FIG. 20), it was hypothesized that in addition to the same decoded and ground-truth phoneme being similar to one another, phonemes with shared acoustic properties would also be characterized as similar to one another. For example, two fricatives will be more acoustically similar to one another than to a vowel.

Hierarchical clustering on the KL-divergence of each phoneme pair demonstrated that phonemes were clustered into four main groups. Group 1 contained consonants with an alveolar place of constriction. Group 2 contained almost all other consonants. Group 3 contained mostly high vowels. Group 4 contained mostly mid and low vowels. The difference between groups tended to correspond to variations along acoustically significant dimensions (frequency range of spectral energy for consonants, and formants for vowels). Indeed, these groupings explain some of the confusions reflected in listener transcriptions of these stimuli. This hierarchical clustering was also consistent with the acoustic similarity matrix of only ground-truth phoneme-pairs (FIG. 6) (cophenetic correlation=0.71, p=1e¹⁰.

Third, since the success of the decoder depends on the initial electrode placement, the contribution of several anatomical regions (vSMC, STG, and IFG) that are involved in continuous speech production was quantified. Decoders were trained in a leave-one-region-out fashion where all electrodes from a particular region were held out (FIG. 17D) and performance was compared. Removing any region led to some decreased decoder performance (FIGS. 17E-17F) (p=3e⁻⁴, n=100, WSRT). However, excluding vSMC resulted in the largest decrease in performance (1.13 dB MCD increase).

Fourth, it was investigated whether the decoder generalized to novel sentences that were never seen in the training data. Since P1 produced some sentences multiple times, two decoders were compared: one that was trained on all sentences (not the particular instances in the test set), and one that was trained excluding every instance of the sentences in the testing set. No significant difference was found in decoding performance of the sentences for both MCD and correlations of spectral features (p=0.36, p=0.75, n=51, WSRT). As a result, the decoder can generalize to arbitrary words and sentences that the decoder was never trained on.

### Silently Mimed Speech Decoding

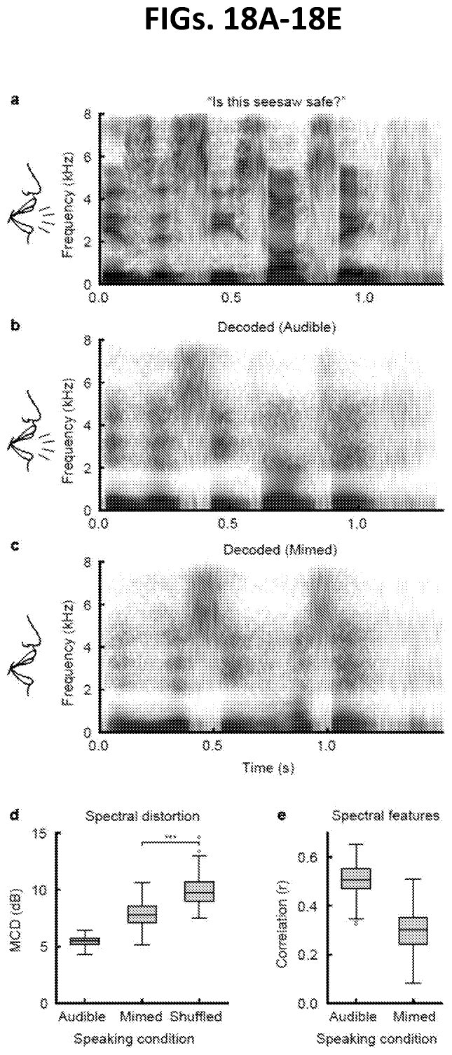

To rule out the possibility that the decoder is relying on the auditory feedback of participants' vocalization, and to simulate a setting where subjects do not overtly vocalize, the decoder was tested on silently mimed speech. A decoder with a held-out set of 58 sentences was tested in which the participant (P1) audibly produced each sentence and then mimed the same sentence, making the same kinematic movements but without making sound. Even though the decoder was not trained on mimed sentences, the spectrograms of synthesized silent speech demonstrated similar spectral patterns to synthesized audible speech of the same sentence (FIGS. 18A-18C). With no original audio to compare, performance of the synthesized mimed sentences was quantified with the audio from the trials with spoken sentences. The spectral distortion and correlation of the spectral features was calculated by first dynamically time-warping the spectrogram of the synthesized mimed speech to match the temporal profile of the audible sentence (FIGS. 18D-18E) and then comparing performance Performance on mimed speech was inferior to that of audible/spoken speech (30% MCD difference), and demonstrates that it is possible to decode important spectral features of speech that were never audibly uttered (p<1e⁻¹¹, compared to chance, n=58; Wilcoxon signed-rank test).

### State-Space of Decoded Speech Articulation

Modeling the underlying kinematics enhances the decoding performance Low-dimensional kinematic state-space trajectories were examined, by computing the state-space projection via principal components analysis (PCA) on the articulatory kinematic features. The first ten principal components (PCs) (of 33 total) captured 85% of the variance and the first two PCs captured 35%.

The state-space trajectories appeared to manifest the dynamics of syllabic patterns in continuous speech. When examining transitions of specific phonemes, it was found that PC1 and PC2 retained their biphasic trajectories of vowel/consonant states, but showed specificity toward particular phonemes indicating that PC1 and PC2 are not necessarily just describing jaw opening and closing, but rather global opening and closing configurations of the vocal tract. These findings are consistent with theoretical accounts of human speaking behavior, which postulate that high-dimensional speech acoustics lie on a low-dimensional articulatory state-space.

To evaluate the similarity of the decoded state-space trajectories, productions of the same sentence across participants that were projected into their respective kinematic state-spaces were correlated (only P1, P2, and P4 had comparable sentences). The state-space trajectories were highly similar (r>0.8, FIG. 18F), demonstrating that the decoder is likely relying upon a shared representation across speakers, a critical basis for generalization.

A shared kinematic representation across speakers could be very advantageous for someone who cannot speak as it may be more intuitive and faster to first learn to use the kinematics decoder (Stage 1), while using an existing kinematics-to-acoustics decoder (stage 2) trained on speech data collected independently.

### Discussion

The results demonstrate intelligible speech synthesis from ECoG during both audible and silently mimed speech production. The present disclosure demonstrates speech synthesis using high-density, direct cortical recordings from human speech cortex. The decoder of the present disclosure explicitly incorporated the knowledge to simplify the translation of neural activity to sound by first decoding the primary physiological correlate of neural activity and then transforming to speech acoustics. This statistical mapping permits generalization with limited amounts of training.

The results show that cortical activity at vSMC electrodes provided for decoding (FIGS. 17E-17F) because it encodes the underlying articulatory physiology that produces speech. This knowledge was incorporated to simply the complex mapping from neural activity to sound by first decoding the physiological correlate of neural activity and then transforming to speech acoustics. Therefore, statistical mapping permits generalization with limited amounts of training

The present disclosure represents one step forward for addressing a major challenge posed by paralyzed patients who cannot speak. The results demonstrate that speakers share a similar kinematic state-space representation (speaker-independent), and it is possible to transfer model knowledge about the mapping of kinematics to sound across subjects. Tapping into this emergent, low-dimensional representation from coordinated population neural activity in the intact cortex may be a critical for bootstrapping a decoder, as well facilitating BCI learning.

TABLE 2
Listener transcriptions of neurally synthesized speech.
Examples shown at several word error rate levels.
The original text is indicated by “o” and the listener
transcriptions are indicated by “t”.

| Word Error | Original sentences (o) and transcriptions |
|---|---|
| Rate | of synthesized speech (t) |
| 0% | o: is this seesaw safe |
| | t: is this seesaw safe |
| ~10% | o: bob bandaged both wounds |
| | with the skill of a doctor |
| | t: bob bandaged full wounds |
| | with the skill of a doctor |
| ~20% | o: those thieves stole thirty jewels |
| | t: thirty thieves stole thirty jewels |
| | o: help celebrate brother's success |
| | t: help celebrate his brother's success |
| ~30% | o: get a calico cat to keep the rodents away |
| | t: the calico cat to keep the rabbits away |
| | o: carl lives in a lively home |
| | t: carl has a lively home |
| ~50% | o: mum strongly dislikes appetizers |
| | t: mom often dislikes appetizers |
| | o: etiquette mandates compliance |
| | with existing regulations |
| | t: etiquette can be made with |
| | existing regulations |
| >70% | o: at twilight on the twelfth |
| | day we'll have Chablis |
| | t: i was walking through chablis |

### Methods

Participants and experimental task. Three human participants (30 F, 31 F, 34 M) underwent chronic implantation of high-density, subdural electrode array over the lateral surface of the brain as part of their clinical treatment of epilepsy (right, left, and right hemisphere grids, respectively). Participants gave their written informed consent before the day of the surgery. All participants were fluent in English. All protocols were approved by the Committee on Human Research at UCSF and experiments/data in this study complied with all relevant ethical regulations. Each participant read and/or freely spoke a variety of sentences. P1 read aloud two complete sets of 460 sentences from the MOCHA-TIMIT database. Additionally, P1 also read aloud passages from the following stories: Sleeping Beauty, Frog Prince, Hare and the Tortoise, The Princess and the Pea, and Alice in Wonderland. P2 read aloud one full set of 460 sentences from the MOCHA-TIMIT database and further read a subset of 50 sentences an additional 9 times each. P3 read 596 sentences describing three picture scenes and then freely described the scene resulting in another 254 sentences. P3 also spoke 743 sentences during free response interviews. In addition to audible speech, P1 also read 10 sentences 12 times each alternating between audible and silently mimed (i.e. making the necessary mouth movements) speech. Microphone recordings were obtained synchronously with the ECoG recordings.

Data acquisition and signal processing. Electrocorticography was recorded with a multi-channel amplifier optically connected to a digital signal processor (Tucker-Davis Technologies). Speech was amplified digitally and recorded with a microphone simultaneously with the cortical recordings. ECoG electrodes were arranged in a 16×16 grid with 4 mm pitch. The grid placements were decided upon purely by clinical considerations. ECoG signals were recorded at a sampling rate of 3,052 Hz. Each channel was visually and quantitatively inspected for artifacts or excessive noise (typically 60 Hz line noise). The analytic amplitude of the high-gamma frequency component of the local field potentials (70-200 Hz) was extracted with the Hilbert transform and down-sampled to 200 Hz. The low frequency component (1-30 Hz) was also extracted with a 5th order Butterworth bandpass filter, down-sampled to 200 Hz and parallelly aligned with the high-gamma amplitude. Finally, the signals were z-scored relative to a 30 second window of running mean and standard deviation, so as to normalize the data across different recording sessions. High-gamma amplitude was studied because it correlates well with multi-unit firing rates and has the temporal resolution to resolve fine articulatory movements. A low frequency signal component was also included due to the decoding performance improvements note for reconstructing perceived speech from auditory cortex. Decoding models were constructed using all electrodes from vSMC, STG, and IFG except for electrodes with bad signal quality as determined by visual inspection.

Phonetic and phonological transcription. For the collected speech acoustic recordings, transcriptions were corrected manually at the word level so that the transcript reflected the vocalization that the participant actually produced. Given sentence level transcriptions and acoustic utterances chunked at the sentence level, hidden Markov model based acoustic models were built for each participant so as to perform sub-phonetic alignment within the Festvox framework. Phonological context features were also generated from the phonetic labels, given their phonetic, syllabic and word contexts.

Cortical surface extraction and electrode visualization. The electrodes were localized on each individual's brain by co-registering the preoperative T1 MRI with a postoperative CT scan containing the electrode locations, using a normalized mutual information routine in SPM12. Pial surface reconstructions were created using Freesurfer. Final anatomical labeling and plotting was performed using the img pipe python package.

Inference of articulatory kinematics. The articulatory kinematics inference model comprises a stacked deep encoder-decoder, where the encoder combines phonological and acoustic representations into a latent articulatory representation that is then decoded to reconstruct the original acoustic signal. The latent representation is initialized with inferred articulatory movement from Electromagnetic Midsagittal Articulography (EMA) and appropriate manner features. A statistical subject-independent approach to acoustic-to-articulatory inversion which estimates 12 dimensional articulatory kinematic trajectories (x and y displacements of tongue dorsum, tongue blade, tongue tip, jaw, upper lip and lower lip, as would be measured by EMA) using only the produced acoustics and phonetic transcriptions is known. Since, EMA features do not describe all acoustically consequential movements of the vocal tract, complementary speech features were appended that improve reconstruction of original speech. In addition to voicing and intensity of the speech signal, place manner tuples were added (represented as continuous binary valued features) to bootstrap the EMA with what was determined were missing physiological aspects in EMA. There were 18 additional values to capture the following place-manner tuples: 1) velar stop, 2) velar nasal, 3) palatal approximant, 4) palatal fricative, 5) palatal affricate, 6) labial stop, 7) labial approximant, 8) labial nasal, 9) glottal fricative, 10) dental fricative, 11) labiodental fricative, 12) alveolar stop, 13) alveolar approximant, 14) alveolar nasal, 15) alveolar lateral, 16) alveolar fricative, 17) unconstructed, 18) voicing. For this purpose, an existing annotated speech database (Wall Street Journal Corpus) was used and trained speaker independent deep recurrent network regression models to predict these place-manner vectors only from the acoustics, represented as 25-dimensional Mel Frequency Cepstral Coefficients (MFCCs). The phonetic labels were used to determine the ground truth values for these labels (e.g., the dimension “labial stop” would be 1 for all frames of speech that belong to the phonemes /p/, /b/ and so forth). However, with a regression output layer, predicted values were not constrained to the binary nature of the input features. In all, these 32 combined feature vectors form the initial articulatory feature estimates.

Finally, to ensure that the combined 32 dimensional representation has the potential to reliably reconstruct speech, an autoencoder was designed to optimize these values. Specifically, a recurrent neural network encoder is trained to convert phonological and acoustic features to the initialized 32 articulatory representations and then a decoder converts the articulatory representation back to the acoustics. The stacked network is re-trained optimizing the joint loss on acoustic and EMA parameters. After convergence, the encoder is used to estimate the final articulatory kinematic features that act as the intermediate to decode acoustics from ECoG.

Neural decoder. The decoder maps ECoG recordings to MFCCs via a two stage process by learning intermediate mappings between ECoG recordings and articulatory kinematic features, and between articulatory kinematic features and acoustic features. All data (ECoG, kinematics, and acoustics) are sampled and processed by the model at 200 Hz. This model was implemented using TensorFlow in python. In the first stage, a stacked 3-layer bLSTM learns the mapping between 300 ms (60 time points) window of high-gamma and LFP signals and a corresponding single time point (sampled at 200 Hz) of the 32 articulatory features. In the second stage, an additional stacked 3-layer bLSTM learns the mapping between the output of the first stage (decoded articulatory features) and 32 acoustic parameters (200 Hz) for full sentences sequences. These parameters are 25 dimensional MFCCs, 5 sub-band voicing strengths for glottal excitation modelling, log(F0), voicing. At each stage, the model is trained to with a learning rate of 0.001 to minimize mean-squared error of the target. Dropout rate is set to 50% to suppress overfitting tendencies of the model. A bLSTM was used because of their ability to retain temporally distant dependencies when decoding a sequence.

During testing, a full sentence sequence of neural activity (high-gamma and low-frequency components) is processed by the decoder. The first stage processes 300 ms of data at a time, sliding over the sequence sample by sample, until it has returned a sequence of kinematics that is equal length to the neural data. The neural data is padded with an additional 150 ms of data before and after the sequence to ensure the result is the correct length. The second stage processes the entire sequence at once, returning an equal length sequence of acoustic features. These features are then synthesized into an audio signal.

At each stage, the model is trained using the Adam optimizer to minimize mean-squared error. The optimizer was initialized with learning rate=0.001,betal=0.9, beta2=0.999, epsilon=1e-8. Models were stopped from training after the validation loss no longer decreased. Dropout rate is set to 50% in stage 1 and 25% in stage 2 to suppress overfitting tendencies of the models. There are 100 hidden units for each LSTM cell. Each model employed 3 stacked bLSTMs with an additional linear layer for regression. A bLSTM was used because of their ability to retain temporally distant dependencies when decoding a sequence.

In the first stage, the batch size for training is 256, and in the second stage the batch size is 25. Training and testing data were randomly split based off of recording sessions, meaning that the test set was collected during separate recording sessions from the training set. The training and testing splits in terms of total speaking time (minutes:seconds) are as follows: P1—training: 92:15, testing: 4:46 (n=101); P2—training: 36:57, testing: 3:50 (n=100); P3—training: 107:42, testing: 4:44 (n=98); P4—training: 27:39, testing 3:12 (n=82); P5—training 44:31, testing 2:51 (n=44). n=number of sentences in test set.

The “direct” ECoG to acoustics decoder a similar architecture as the stage 1 articulatory bLSTM except with an MFCC output. Originally the direct acoustic decoder was trained as a 6-layer bLSTM that mimics the architecture of the 2 stage decoder with MFCCs as the “intermediate layer” and as the output. However, it was found that performance was better with a 4-layer bLSTM (no intermediate layer) with 100 hidden units for each layer, 50% dropout and 0.005 learning rate using Adam optimizer for minimizing mean-squared error. Models were coded using Python's version 1.9 of Tensorflow.

Speech synthesis from acoustic features. An implementation of the Mel-log spectral approximation algorithm with mixed excitation within Festvox was used to generate the speech waveforms from estimates of the acoustic features from the neural decoder.

Model training procedure. As described, simultaneous recordings of ECoG and speech are collected in short blocks of approximately 5 minutes. To partition the data for model development, 2-3 blocks were allocated for model testing, 1 block for model optimization, and the remaining blocks for model training. The test sentences 432 for P1 and P2 each spanned 2 recording blocks and comprised 100 sentences read aloud. The test sentences for P3 were different because the speech comprised 100 sentences over three blocks of freely and spontaneously speech describing picture scenes. For shuffling the data to test for significance, the order of the electrodes were shuffled that were fed into the decoder. This method of shuffling preserved the temporal structure of the neural activity.

Mel-Cepstral Distortion (MCD). To examine the quality of synthesized speech, the Mel-Cepstral Distortion (MCD) of the synthesized speech was calculated when compared the original ground-truth audio. MCD is an objective measure of error determined from MFCCs and is correlated to subjective perceptual judgments of acoustic quality. For reference acoustic features mc^{(y)} and decoded features mc^{(ŷ)},

$$M C D = \frac{10}{ln ( 10 )} \sqrt{{\sum }_{0 < d < 25} {( m {c}_{d}^{( y )} - m {c}_{d}^{( \hat{y} )} )}^{2}} \qquad ( 1 )$$

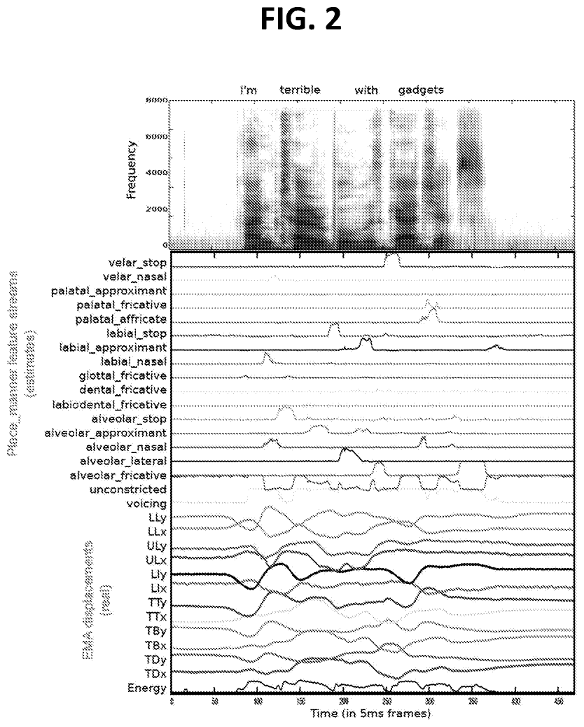

Intelligibility Assessment. Listening tests using crowdsourcing are a standard way of evaluating the perceptual quality of synthetic speech. To comprehensively assess the intelligibility of the neurally synthesized speech, a series of identification and transcription tasks was conducted on the Amazon Mechanical Turk. A set of 60 sentences (6 trials of 10 unique sentences) were evaluated int his assessment. These trials, also held out during training the decoder, were used in place of the 100 unique sentences tested throughout the rest of FIG. 2 because the listeners always had the same 10 sentences to chose from. Each trial sentence was listened to by 50 different listeners. In all, 166 unique listeners took part in the evaluations.

To assess the amount of training data affects decoder performance, the data was partitioned by recording blocks and trained a separate model for an allotted number of blocks. In total, 8 models were trained, each with one of the following block allotments: [1, 2, 5, 10, 15, 20, 25, 28]. Each block comprised an average of 50 sentences recorded in one continuous session.

For the word level identification tasks, several cohorts of words grouped by the number of syllables within were created. Using the time boundaries from the ground truth phonetic labelling, audio was extracted from the neurally synthesized speech into four classes of 1-syllable, 2-syllable, 3-syllable and 4-syllable words. Tests were conducted on each of these groups of words that involve identification of the synthesized audio from a group of i) 10 choices, ii) 25 choices, and iii) 50 choices of what they think the word is. The presented options included the true word and the remaining choices randomly drawn from the other words within the class. All words within the word groups were judged for intelligibility without any further sub-selection.

Since the content words in the MOCHA-TIMIT data are largely low frequency words to assess sentence-level intelligibility, along with the neurally synthesized audio file, the listeners were presented with a pool of words that may be in the sentence. This makes it task a limited vocabulary free response transcription. Two experiments were conducted where the transcriber is presented with pool of i) 25 word choices, and ii) 50 word choices that may be used the sentence. The true words that make up the sentence are included along with randomly drawn words from the entire test set and displayed in alphabetical order. Given that the median sentence is only 7 words long (std=21, min=4, max=13), this task design allows for reliable assessment of intelligibility. Each trial was judged by 10-20 different listeners. Each intelligibility task was performed by 47-187 unique listeners (a total of 1755 listeners across 16 intelligibility tasks making all reported analyses statistically reliable. All sentences from the test set were sent for intelligibility assessment without any further selection. The listeners were required to be English speakers located in the United States, with good ratings(>98% rating from prior tasks on the platform). For the sentence transcription tasks, an automatic spell checker was employed to correct misspellings. No further spam detection, or response rejection was done in all analyses reported. Word Error Rate (WER) metric computed on listener transcriptions is used to judge the intelligibility of the neurally synthesized speech. Where I is the number of word insertions, D is the number of word deletions and S is the number of word substitutions for a reference sentence with N words, WER is computed as

$$W E R = \frac{I + D + S}{N} \qquad ( 2 )$$

Data limitation analysis. To assess the amount of training data affects decoder performance, the data was partitioned by recording blocks and trained a separate model for an allotted number of blocks. In total, 8 models were trained, each with one of the following block allotments: [1, 2, 5, 10, 15, 20, 25, 28]. Each block comprised an average of 50 sentences recorded in one continuous session.

Quantification of silent speech synthesis. By definition, there was no acoustic signal to compare the decoded silent speech. In order to assess decoding performance, decoded silent speech was evaluated in regards to the audible speech of the same sentence uttered immediately prior to the silent trial. This was done so dynamically time-warping the decoded silent speech MFCCs to the MFCCs of the audible condition and computing Pearson's correlation coefficient and Mel-cepstral distortion.

Phoneme acoustic similarity analysis. The acoustic properties of decoded phonemes were compared to ground-truth to better understand the performance of the decder of the present disclosure. To do this, all time points were sliced for which a given phoneme was being uttered and used the corresponding time slices to estimate its distribution of spectral properties. With principal components analysis (PCA), the 32 spectral features were projected onto the first 4 principal components before fitting the gaussian kernel density estimate (KDE) model. This process was repeated so that each phoneme had two KDEs representing either its decoded and or ground-truth spectral properties. Using Kullback-Leibler divergence (KL divergence), each decoded phoneme KDE was compared to every ground-truth phoneme KDE, creating an analog to a confusion matrix used in discrete classification decoders. KL divergence provides a metric of how similar two distributions are to one another by calculating how much information is lost when one distribution was approximated with another. Lastly, Ward's method was used for agglomerative hierarchical clustering to organize the phoneme similarity matrix.

To understand whether the clustering of the decoded phonemes was similar to the clustering of ground-truth phoneme pairs, the cophenetic correlation (CC) was used to assess how well the hierarchical clustering determined from decoded phonemes preserved the pairwise distance between original phonemes, and vice versa²⁴. For the decoded phoneme dendrogram, the CC for preserving original phoneme distances was 0.71 as compared to 0.80 for preserving decoded phoneme distances. For the original phoneme dendrogram, the CC for preserving decoded phoneme distances was 0.64 as compared to 0.71 for preserving original phoneme distances. p<1e-10 for all correlations.

State-space kinematic trajectories. For state-space analysis of kinematic trajectories, principal components analysis (PCA) was performed on the 33 kinematic features using the training data set from P1. FIGS. 4A-4B shows kinematic trajectories (original, decoded (audible and mimed) projected onto the first two principal components (PCs). The example decoded mimed trajectory occurred faster in time by a factor of 1.15 than the audible trajectory so the trajectory was uniformly temporally stretched for visualization. The peaks and troughs of the decoded mimed trajectories were similar to the audible speech trajectory (r=0.65, r=0.55) although the temporal locations are shifted relative to one another, likely because the temporal evolution of a production, whether audible or mimed, is inconsistent across repeated productions. To quantify the decoding performance of mimed trajectories, the dynamic time-warping approach described above was used, although in this case, temporally warping with respect to the inferred kinematics (not the state-space).

For analysis of state-space trajectories across participants, the correlations of productions of the same sentence were measured, but across participants. Since the sentences were produced at different speeds, they were dynamically time-warped to match and compared against correlations of dynamically time-warped mismatched sentences.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. §112(6) is not invoked.

What is claimed is:

1. A method comprising:
receiving a physiological feature signal associated with a spatiotemporal movement of a vocal tract articulator;
generating a speech pattern signal in response to the physiological feature signal; and
outputting speech that is based on the speech pattern signal.

2. The method according to claim 1, wherein the vocal tract articulator is selected from the group consisting of the upper lip, lower lip, lower incisor, tongue tip, tongue blade, tongue dorsum and larynx.

3. The method according to any one of claims 1-2, wherein the physiological feature signal comprises measurements of the caudo-rostral displacements of one or more of the vocal tract articulators.

4. The method according to claim any one of claims 1-3, wherein the method comprises measuring the caudo-rostral displacements of one or more of the vocal tract articulators associated with consonant constriction.

5. The method according to claim 4, wherein the consonant is plosive, lateral, fricative or nasal.

6. The method according to any one of claims 1-5, wherein spatiotemporal movement of a vocal tract articulator is measured by electromagnetic midsagittal articulography.

7. The method according to claim 1, wherein receiving a physiological feature signal comprises:
receiving one or more brain signals; and
associating the brain signals to one or more of the spatiotemporal movements of a vocal tract articulator.

8. The method according to claim 9, wherein the signals are detected from the ventral sensorimotor cortex of the brain.

9. The method according to claim 3, wherein the caudo-rostral displacements are configured to measure the shape and location of the one or more vocal tract articulators.

10. The method according to any one of claims 1-12, wherein the speech pattern signal is outputted as auditory speech or as text.

11. A method comprising:
acquiring one or more of:
a linguistic signal; and
an acoustic signal;
associating a physiological feature with the linguistic or acoustic signal;
generating a speech pattern signal in response to the physiological feature; and
outputting speech that is based on the speech pattern signal.

12. The method according to claim 11, wherein the linguistic signal is a lexical signal.

13. The method according to any one of claims 11-12, wherein the linguistic signal is a phonological signal.

14. The method according to any one of claims 11-13, wherein associating a physiological feature with the linguistic or acoustic signal comprises associating the linguistic or acoustic signal with a spatiotemporal movement of a vocal tract articulator.

15. The method according to claim 14, wherein the vocal tract articulator is selected from the group consisting of the upper lip, lower lip, lower incisor, tongue tip, tongue blade, tongue dorsum and larynx.

16. The method according to any one of claims 14-15, wherein the method comprises measuring the caudo-rostral displacements of one or more of the vocal tract articulators.

17. The method according to claim any one of claims 14-16, wherein the method comprises measuring the caudo-rostral displacements of one or more of the vocal tract articulators associated with consonant constriction.

18. The method according to claim 17, wherein the consonant is plosive, lateral, fricative or nasal.

19. The method according to any one of claims 14-18, wherein spatiotemporal movement of a vocal tract articulator is measured by electromagnetic midsagittal articulography.

20. The method according to any one of claims 11-19, wherein associating a physiological feature with the linguistic or acoustic signal further comprises:
detecting one or more signals from the brain; and
associating the brain signals to one or more spatiotemporal movements of a vocal tract articulator.

21. The method according to claim 20, wherein the signals are detected from the ventral sensorimotor cortex of the brain.

22. The method according to claim 20, wherein the vocal tract articulator is selected from the group consisting of the upper lip, lower lip, lower incisor, tongue tip, tongue blade, tongue dorsum and larynx.

23. The method according to any one of claims 11-22, wherein the speech signal is outputted as auditory speech or as text.

24. A system comprising:
a processor comprising memory operably coupled to the processor wherein the memory includes instructions stored thereon, which when executed by the processor, cause the processor to:
receive a physiological feature signal associated with a spatiotemporal movement of a vocal tract articulator; and
generate a speech pattern signal in response to the physiological feature signal; and
an output for outputting speech that is based on the speech pattern signal.

25. The system according to claim 24, wherein the processor comprises bidirectional long-short term memory (bLSTM).

26. The system according to claim 25, wherein the bLSTM is a stacked 3-layer b1STM processor configured to encode one or more vocal tract articulators.

27. The system according to claim 25, wherein the bidirectional long-short term memory comprises algorithm for encoding the physiological feature signal.

28. The system according to any one of claims 24-27, wherein the processor comprises a deep neural network (DNN).

29. The system according to claim 28, wherein the deep neural network comprises algorithm for decoding the physiological feature signal to a speech pattern signal.

30. The system according to claim 29, wherein the deep neural network comprises algorithm for decoding physiological signal to auditory speech.

31. The system according to claim 29, wherein the deep neural network comprises algorithm for decoding physiological signal to text.

32. The system according to any one of claims 28-31, wherein the deep neural network comprises algorithm for decoding physiological signal as mel frequency cepstral coefficients.

33. The system according to claim 32, wherein the deep neural network comprises algorithm for decoding physiological signal as 25 dimensional mel frequency cepstral coefficients.

34. The system according to any one of claims 24-33, wherein the physiological feature signal comprises a dataset associated with spatiotemporal movement of one or more vocal tract articulators.

35. The system according to claim 34, wherein the vocal tract articulator is selected from the group consisting of the upper lip, lower lip, lower incisor, tongue tip, tongue blade, tongue dorsum and larynx.

36. The system according to claim 34, wherein the dataset comprises measurements of the caudo-rostral displacements of the one or more of the vocal tract articulators.

37. The system according to claim 36, wherein the physiological feature comprises a electromagnetic midsagittal articulography dataset associated with spatiotemporal movement of one or more vocal tract articulators.

38. The system according to any one of claims 24-37, further comprising memory operably coupled to the processor wherein the memory includes instructions stored thereon, which when executed by the processor, cause the processor to:
receive one or more signals from the brain; and
associate the brain signals to one or more spatiotemporal movements of a vocal tract articulator to generate a physiological feature signal; and
generate a speech pattern signal in response to the physiological feature signal.

39. The system according to claim 38, further comprising electrical leads for receiving signals from all or a part of the ventral sensorimotor cortex of the brain.

40. The system according to any one of claims 24-39, wherein the output is configured to output auditory speech or text.

41. The system according to claim 40, wherein the output is an audio speaker.

42. The system according to claim 40, wherein the output is a text generator.

43. A system comprising:
input for receiving one or more of:
a linguistic signal; and
an acoustic signal; and
a processor comprising memory operably coupled to the processor wherein the memory includes instructions stored thereon, which when executed by the processor, cause the processor to:
associate a physiological feature with an inputted linguistic or acoustic signal; and
an output configured to output a speech signal in response to the physiological feature.

44. The system according to claim 43, wherein the processor comprises bidirectional long-short term memory (BLSTM).

45. The system according to claim 44, wherein the bidirectional long-short term memory comprises algorithm for encoding the physiological signal associated with the inputted linguistic or acoustic signal.

46. The system according to any one of claims 43-45, wherein the processor comprises a deep neural network (DNN).

47. The system according to claim 46, wherein the deep neural network comprises algorithm for decoding physiological signal to a speech signal.

48. The system according to claim 47, wherein the deep neural network comprises algorithm for decoding physiological signal to auditory speech.

49. The system according to claim 48, wherein the deep neural network comprises algorithm for decoding physiological signal to text.

50. The system according to any one of claims 46-49, wherein the deep neural network comprises algorithm for decoding physiological signal as mel frequency cepstral coefficients.

51. The system according to claim 50, wherein the deep neural network comprises algorithm for decoding physiological signal as 25 dimensional mel frequency cepstral coefficients.

52. The system according to any one of claims 43-51, wherein the physiological feature comprises a dataset associated with spatiotemporal movement of one or more vocal tract articulators.

53. The system according to claim 52, wherein the vocal tract articulator is selected from the group consisting of the upper lip, lower lip, lower incisor, tongue tip, tongue blade, tongue dorsum and larynx.

54. The system according to claim 53, wherein the dataset comprises measurements of the caudo-rostral displacements of the one or more of the vocal tract articulators.

55. The system according to claim 54, wherein the physiological feature comprises a electromagnetic midsagittal articulography dataset associated with spatiotemporal movement of one or more vocal tract articulators.

56. The system according to any one of claims 43-51, further comprising memory operably coupled to the processor wherein the memory includes instructions stored thereon, which when executed by the processor, cause the processor to:
receive one or more signals from the brain; and
associate the brain signals to one or more spatiotemporal movements of a vocal tract articulator to generate a physiological feature signal; and
generate a speech pattern signal in response to the physiological feature signal.

57. The system according to claim 56, further comprising electrical leads for receiving signals from all or a part of the ventral sensorimotor cortex of the brain.

58. The system according to any one of claims 43-57, wherein the output is configured to output auditory speech or text.

59. The system according to claim 58, wherein the output is an audio speaker.

60. The system according to claim 58, wherein the output is a text generator.
